(19) United States
(12) Patent Application Publication
(10) Pub. No.: US 2023/0272446 A1
(43) Pub. Date: Aug. 31, 2023

(54) Repeat Protein Architectures

(71) Applicant: University of Washington, Seattle, WA (US)

(72) Inventors: Fabio PARMEGGIANI, Seattle, WA (US); TJ BRUNETTE, Seattle, WA (US); Po-Ssu HUANG, Seattle, WA (US); David BAKER, Seattle, WA (US)

(21) Appl. No.: 18/312,788

(22) Filed: May 5, 2023

Related U.S. Application Data
(63) Continuation-in-part of application No. 16/060,640, filed on Jun. 8, 2018, now abandoned.
(60) Provisional application No. 62/268,320, filed on Dec. 16, 2015.

(51) Int. Cl. C12P 21/02; C07K 14/00; C12N 15/70

(52) U.S. Cl. CPC C12P 21/02; C07K 14/001; C12N 15/70; C12N 2800/101

(57) ABSTRACT

Methods and systems for designing proteins are disclosed, as well as proteins and protein assemblies designed. A computing device can determine a protein repeating unit that includes one or more protein helices and one or more protein loops. The computing device can generate a protein backbone structure with a copy of the protein repeating unit. The computing device can determine whether a distance between a pair of helices of the protein backbone structure is between lower and upper distance thresholds. After determining that the distance between the pair of helices is between the lower and upper distance thresholds, the computing device can: generate a plurality of protein sequences based on the protein backbone structure, select a particular protein sequence of the plurality of protein sequences based on an energy landscape that has information about energy and distance from a target fold of the particular protein sequence, and generate an output based on the particular protein sequence.

### STATEMENT REGARDING FEDERALLY SPONSORED RESEARCH

This invention was made with U.S. government support under MCB-1445201 and CHE-1332907, awarded by National Science Foundation, under N00024-10-D-6318/0024, awarded by the Defense Threat Reduction Agency, and under FA950-12-10112, awarded by the Air Force Office of Scientific Research. The U.S. Government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Apr. 27, 2023 having the file name “16-1442-WO-US-CIP.xml” and is 3,486,771 bytes in size.

### BACKGROUND

A central question in protein evolution is the extent to which naturally occurring proteins sample the space of folded structures accessible to the polypeptide chain. Repeat proteins composed of multiple tandem copies of a modular structure unit¹ are widespread in nature and play critical roles in molecular recognition, signaling, and other essential biological processes². Naturally occurring repeat proteins have been reengineered for molecular recognition and modular scaffolding applications³⁻⁵.

### SUMMARY OF THE INVENTION

Here we use computational protein design to investigate the space of folded structures that can be generated by tandem repeating a simple helix-loop-helix-loop structural motif 83 designs with sequences unrelated to known repeat proteins were experimentally characterized. 53 were monomeric and stable at 95° C., and 43 have solution x-ray scattering spectra closely consistent with the design models. Crystal structures of 15 designs spanning a broad range of curvatures are in close agreement with the design models with RMSDs ranging from 0.7 to 2.5 Å. Our results show that existing repeat proteins occupy only a small fraction of the possible repeat protein sequence and structure space and that it is possible to design novel repeat proteins with precisely specified geometries, opening up a wide array of new possibilities for biomolecular engineering.

In one aspect, the present invention provides polypeptides comprising or consisting of the amino acid sequence selected from the group consisting of the following multi-domain proteins, as further defined in the detailed description:
(a) SEQ ID NO:1-[SEQ ID NO:2]_{(0 or 2-19)}-SEQ ID NO:3;
(b) SEQ ID NO:7-[SEQ ID NO:8]_{(0 or 2-19)}-SEQ ID NO:9;
(c) SEQ ID NO:13-[SEQ ID NO:14]_{(0 or 2-19)}-SEQ ID NO:15;
(d) SEQ ID NO:19-[SEQ ID NO:20]_{(0 or 2-19)}-SEQ ID NO:21;
(e) SEQ ID NO:25-[SEQ ID NO:26]_{(0 or 2-19)}-SEQ ID NO:27;
(f) SEQ ID NO:31-[SEQ ID NO:32]_{(0 or 2-19)}-SEQ ID NO:33;
(g) SEQ ID NO:37-[SEQ ID NO:38]_{(0 or 2-19)}-SEQ ID NO:39;
(h) SEQ ID NO:43-[SEQ ID NO:44]_{(0 or 2-19)}-SEQ ID NO:45;
(i) SEQ ID NO:49-[SEQ ID NO:50]_{(0 or 2-19)}-SEQ ID NO:51;
(j) SEQ ID NO:55-[SEQ ID NO:56]_{(0 or 2-19)}-SEQ ID NO:57;
(k) SEQ ID NO:61-[SEQ ID NO:62]_{(0 or 2-19)}-SEQ ID NO:63;
(l) SEQ ID NO:67-[SEQ ID NO:68]_{(0 or 2-19)}-SEQ ID NO:69;
(m) SEQ ID NO:73-[SEQ ID NO:74]_{(0 or 2-19)}-SEQ ID NO:75;
(n) SEQ ID NO:79-[SEQ ID NO:80]_{(0 or 2-19)}-SEQ ID NO:81;
(o) SEQ ID NO:85-[SEQ ID NO:86]_{(0 or 2-19)}-SEQ ID NO:87;
(p) SEQ ID NO:91-[SEQ ID NO:92]_{(0 or 2-19)}-SEQ ID NO:93;
(q) SEQ ID NO:97-[SEQ ID NO:98]_{(0 or 2-19)}-SEQ ID NO:99;
(r) SEQ ID NO:103-[SEQ ID NO:104]_{(0 or 2-19)}-SEQ ID NO:105;
(s) SEQ ID NO:109-[SEQ ID NO:110]_{(0 or 2-19)}-SEQ ID NO:111;
(t) SEQ ID NO:115-[SEQ ID NO:116]_{(0 or 2-19)}-SEQ ID NO:117;
(u) SEQ ID NO:121-[SEQ ID NO:122]_{(0 or 2-19)}-SEQ ID NO:123;
(v) SEQ ID NO:127-[SEQ ID NO:128]_{(0 or 2-19)}-SEQ ID NO:129;
(w) SEQ ID NO:133-[SEQ ID NO:134]_{(0 or 2-19)}-SEQ ID NO:135;
(x) SEQ ID NO:139-[SEQ ID NO:140]_{(0 or 2-19)}-SEQ ID NO:141;
(y) SEQ ID NO:145-[SEQ ID NO:146]_{(0 or 2-19)}-SEQ ID NO:147;
(z) SEQ ID NO:151-[SEQ ID NO:152]_{(0 or 2-19)}-SEQ ID NO:153;
(aa) SEQ ID NO: 157-[SEQ ID NO: 158]_{(0 or 2-19)}-SEQ ID NO:159;
(bb) SEQ ID NO:163-[SEQ ID NO:164]_{(0 or 2-19)}-SEQ ID NO:165;
(cc) SEQ ID NO:169-[SEQ ID NO:170]_{(0 or 2-19)}-SEQ ID NO:171;
(dd) SEQ ID NO:175-[SEQ ID NO:176]_{(0 or 2-19)}-SEQ ID NO:177;
(ee) SEQ ID NO:181-[SEQ ID NO:182]_{(0 or 2-19)}-SEQ ID NO:183;
(ff) SEQ ID NO:187-[SEQ ID NO:188]_{(0 or 2-19)}-SEQ ID NO:189;
(gg) SEQ ID NO:193-[SEQ ID NO:194]_{(0 or 2-19)}-SEQ ID NO:195;
(hh) SEQ ID NO:199-[SEQ ID NO:200]_{(0 or 2-19)}-SEQ ID NO:201;
(ii) SEQ ID NO:205-[SEQ ID NO:206]_{(0 or 2-19)}-SEQ ID NO:207;
(jj) SEQ ID NO:211-[SEQ ID NO:212]_{(0 or 2-19)}-SEQ ID NO:213;
(kk) SEQ ID NO:217-[SEQ ID NO:218]_{(0 or 2-19)}-SEQ ID NO:219;
(ll) SEQ ID NO:223-[SEQ ID NO:224]_{(0 or 2-19)}-SEQ ID NO:225;
(mm) SEQ ID NO:229-[SEQ ID NO:230]_{(0 or 2-19)}-SEQ ID NO:231;
(nn) SEQ ID NO:235-[SEQ ID NO:236]_{(0 or 2-19)}-SEQ ID NO:237;
(oo) SEQ ID NO:241-[SEQ ID NO:242]_{(0 or 2-19)}-SEQ ID NO:243;
(pp) SEQ ID NO:247-[SEQ ID NO:248]_{(0 or 2-19)}-SEQ ID NO:249;
(qq) SEQ ID NO:253-[SEQ ID NO:254]_{(0 or 2-19)}-SEQ ID NO:255;
(rr) SEQ ID NO:259-[SEQ ID NO:260]_{(0 or 2-19)}-SEQ ID NO:261;
(ss) SEQ ID NO:265-[SEQ ID NO:266]_{(0 or 2-19)}-SEQ ID NO:267;
(tt) SEQ ID NO:271-[SEQ ID NO:272]_{(0 or 2-19)}-SEQ ID NO:273;
(uu) SEQ ID NO:277-[SEQ ID NO:278]_{(0 or 2-19)}-SEQ ID NO:278;
(vv) SEQ ID NO:283-[SEQ ID NO:284]_{(0 or 2-19)}-SEQ ID NO:285;
(ww) SEQ ID NO:289-[SEQ ID NO:290]_{(0 or 2-19)}-SEQ ID NO:291;
(xx) SEQ ID NO:295-[SEQ ID NO:296]_{(0 or 2-19)}-SEQ ID NO:297;
(yy) SEQ ID NO:301-[SEQ ID NO:302]_{(0 or 2-19)}-SEQ ID NO:303;
(zz) SEQ ID NO:307-[SEQ ID NO:308]_{(0 or 2-19)}-SEQ ID NO:309;
(aaa) SEQ ID NO:313-[SEQ ID NO:314]_{(0 or 2-19)}-SEQ ID NO:315;
(bbb) SEQ ID NO:319-[SEQ ID NO:320]_{(0 or 2-19)}-SEQ ID NO:321;
(ccc) SEQ ID NO:325-[SEQ ID NO:326]_{(0 or 2-19)}-SEQ ID NO:327;
(ddd) SEQ ID NO:331-[SEQ ID NO:332]_{(0 or 2-19)}-SEQ ID NO:333;
(eee) SEQ ID NO:337-[SEQ ID NO:338]_{(0 or 2-19)}-SEQ ID NO:339;
(fff) SEQ ID NO:343-[SEQ ID NO:344]_{(0 or 2-19)}-SEQ ID NO:345;
(ggg) SEQ ID NO:349-[SEQ ID NO:350]_{(0 or 2-19)}-SEQ ID NO:351;
(hhh) SEQ ID NO:355-[SEQ ID NO:356]_{(0 or 2-19)}-SEQ ID NO:357;
(iii) SEQ ID NO:361-[SEQ ID NO:362]_{(0 or 2-19)}-SEQ ID NO:363;
(jjj) SEQ ID NO:367-[SEQ ID NO:368]_{(0 or 2-19)}-SEQ ID NO:369;
(kkk) SEQ ID NO:373-[SEQ ID NO:374]_{(0 or 2-19)}-SEQ ID NO:375;
(lll) SEQ ID NO:379-[SEQ ID NO:380]_{(0 or 2-19)}-SEQ ID NO:381;
(mmm) SEQ ID NO:385-[SEQ ID NO:386]_{(0 or 2-19)}-SEQ ID NO:387;
(nnn) SEQ ID NO:391-[SEQ ID NO:392]_{(0 or 2-19)}-SEQ ID NO:393;
(ooo) SEQ ID NO:397-[SEQ ID NO:398]_{(0 or 2-19)}-SEQ ID NO:399;
(ppp) SEQ ID NO:403-[SEQ ID NO:404]_{(0 or 2-19)}-SEQ ID NO:405; and
(qqq) SEQ ID NO:409-[SEQ ID NO:410]_{(0 or 2-19)}-SEQ ID NO:411;
wherein the domain in brackets is an optional internal domain.

In one embodiment, polypeptide comprises or consists of the amino acid sequence selected from the group consisting of:
(A) SEQ ID NO:4-[SEQ ID NO:5]_{(0 or 2-19)}-SEQ ID NO:6;
(B) SEQ ID NO:10-[SEQ ID NO:11]_{(0 or 2-19)}-SEQ ID NO:12;
(C) SEQ ID NO:16-[SEQ ID NO:17]_{(0 or 2-19)}-SEQ ID NO:18;
(D) SEQ ID NO:22-[SEQ ID NO:23]_{(0 or 2-19)}-SEQ ID NO:24;
(E) SEQ ID NO:28-[SEQ ID NO:29]_{(0 or 2-19)}-SEQ ID NO:30;
(F) SEQ ID NO:34-[SEQ ID NO:35]_{(0 or 2-19)}-SEQ ID NO:36;
(G) SEQ ID NO:40-[SEQ ID NO:41]_{(0 or 2-19)}-SEQ ID NO:42;
(H) SEQ ID NO:46-[SEQ ID NO:47]_{(0 or 2-19)}-SEQ ID NO:48;
(I) SEQ ID NO:52-[SEQ ID NO:53]_{(0 or 2-19)}-SEQ ID NO:54;
(J) SEQ ID NO:58-[SEQ ID NO:59]_{(0 or 2-19)}-SEQ ID NO:60;
(K) SEQ ID NO:64-[SEQ ID NO:65]_{(0 or 2-19)}-SEQ ID NO:66;
(L) SEQ ID NO:70-[SEQ ID NO:71]_{(0 or 2-19)}-SEQ ID NO:72;
(M) SEQ ID NO:76-[SEQ ID NO:77]_{(0 or 2-19)}-SEQ ID NO:78;
(N) SEQ ID NO:82-[SEQ ID NO:83]_{(0 or 2-19)}-SEQ ID NO:84;
(O) SEQ ID NO:88-[SEQ ID NO:89]_{(0 or 2-19)}-SEQ ID NO:90;
(P) SEQ ID NO:94-[SEQ ID NO:95]_{(0 or 2-19)}-SEQ ID NO:96;
(Q) SEQ ID NO:100-[SEQ ID NO:101]_{(0 or 2-19)}-SEQ ID NO:102;
(R) SEQ ID NO:106-[SEQ ID NO:107]_{(0 or 2-19)}-SEQ ID NO:108;
(S) SEQ ID NO:112-[SEQ ID NO:113]_{(0 or 2-19)}-SEQ ID NO:114;
(T) SEQ ID NO:118-[SEQ ID NO:119]_{(0 or 2-19)}-SEQ ID NO:120;
(U) SEQ ID NO:124-[SEQ ID NO:125]_{(0 or 2-19)}-SEQ ID NO:126;
(V) SEQ ID NO:130-[SEQ ID NO:131]_{(0 or 2-19)}-SEQ ID NO:132;
(W) SEQ ID NO:136-[SEQ ID NO:137]_{(0 or 2-19)}-SEQ ID NO:138;
(X) SEQ ID NO:142-[SEQ ID NO:143]_{(0 or 2-19)}-SEQ ID NO:144;
(Y) SEQ ID NO:148-[SEQ ID NO:149]_{(0 or 2-19)}-SEQ ID NO:150;
(Z) SEQ ID NO:154-[SEQ ID NO:155]_{(0 or 2-19)}-SEQ ID NO:156;
(AA) SEQ ID NO:160-[SEQ ID NO:161]_{(0 or 2-19)}-SEQ ID NO:162;
(BB) SEQ ID NO:166-[SEQ ID NO:167]_{(0 or 2-19)}-SEQ ID NO:168;
(CC) SEQ ID NO:172-[SEQ ID NO:173]_{(0 or 2-19)}-SEQ ID NO:174;
(DD) SEQ ID NO:178-[SEQ ID NO:179]_{(0 or 2-19)}-SEQ ID NO:180;
(EE) SEQ ID NO:184-[SEQ ID NO:185]_{(0 or 2-19)}-SEQ ID NO:186;
(FF) SEQ ID NO:190-[SEQ ID NO:191]_{(0 or 2-19)}-SEQ ID NO:192;
(GG) SEQ ID NO:196-[SEQ ID NO:197]_{(0 or 2-19)}-SEQ ID NO:198;
(HH) SEQ ID NO:202-[SEQ ID NO:203]_{(0 or 2-19)}-SEQ ID NO:204;
(II) SEQ ID NO:208-[SEQ ID NO:209]_{(0 or 2-19)}-SEQ ID NO:210;
(JJ) SEQ ID NO:214-[SEQ ID NO:215]_{(0 or 2-19)}-SEQ ID NO:216;
(KK) SEQ ID NO:220-[SEQ ID NO:221]_{(0 or 2-19)}-SEQ ID NO:222;
(LL) SEQ ID NO:226-[SEQ ID NO:227]_{(0 or 2-19)}-SEQ ID NO:228;
(MM) SEQ ID NO:232-[SEQ ID NO:233]_{(0 or 2-19)}-SEQ ID NO:234;
(NN) SEQ ID NO:238-[SEQ ID NO:239]_{(0 or 2-19)}-SEQ ID NO:240;
(OO) SEQ ID NO:244-[SEQ ID NO:245]_{(0 or 2-19)}-SEQ ID NO:246;
(PP) SEQ ID NO:250-[SEQ ID NO:251]_{(0 or 2-19)}-SEQ ID NO:252;
(QQ) SEQ ID NO:256-[SEQ ID NO:257]_{(0 or 2-19)}-SEQ ID NO:258;
(RR) SEQ ID NO:262-[SEQ ID NO:263]_{(0 or 2-19)}-SEQ ID NO:264;
(SS) SEQ ID NO:268-[SEQ ID NO:269]_{(0 or 2-19)}-SEQ ID NO:270;
(TT) SEQ ID NO:274-[SEQ ID NO:275]_{(0 or 2-19)}-SEQ ID NO:276;
(UU) SEQ ID NO:280-[SEQ ID NO:281]_{(0 or 2-19)}-SEQ ID NO:282;
(VV) SEQ ID NO:286-[SEQ ID NO:287]_{(0 or 2-19)}-SEQ ID NO:288;
(WW) SEQ ID NO:292-[SEQ ID NO:293]_{(0 or 2-19)}-SEQ ID NO:294;
(XX) SEQ ID NO:298-[SEQ ID NO:299]_{(0 or 2-19)}-SEQ ID NO:300;
(YY) SEQ ID NO:304-[SEQ ID NO:305]_{(0 or 2-19)}-SEQ ID NO:306;
(ZZ) SEQ ID NO:310-[SEQ ID NO:311]_{(0 or 2-19)}-SEQ ID NO:312;
(AAA) SEQ ID NO:316-[SEQ ID NO:317]_{(0 or 2-19)}-SEQ ID NO:318;
(BBB) SEQ ID NO:322-[SEQ ID NO:323]_{(0 or 2-19)}-SEQ ID NO:324;
(CCC) SEQ ID NO:328-[SEQ ID NO:329]_{(0 or 2-19)}-SEQ ID NO:330;
(DDD) SEQ ID NO:334-[SEQ ID NO:335]_{(0 or 2-19)}-SEQ ID NO:336;
(EEE) SEQ ID NO:340-[SEQ ID NO:341]_{(0 or 2-19)}-SEQ ID NO:342;
(FFF) SEQ ID NO:346-[SEQ ID NO:347]_{(0 or 2-19)}-SEQ ID NO:348;
(GGG) SEQ ID NO:352-[SEQ ID NO:353]_{(0 or 2-19)}-SEQ ID NO:354;
(HHH) SEQ ID NO:358-[SEQ ID NO:359]_{(0 or 2-19)}-SEQ ID NO:360;
(III) SEQ ID NO:364-[SEQ ID NO:365]_{(0 or 2-19)}-SEQ ID NO:366;
(JJJ) SEQ ID NO:370-[SEQ ID NO:371]_{(0 or 2-19)}-SEQ ID NO:372;
(KKK) SEQ ID NO:376-[SEQ ID NO:377]_{(0 or 2-19)}-SEQ ID NO:378;
(LLL) SEQ ID NO:382-[SEQ ID NO:383]_{(0 or 2-19)}-SEQ ID NO:384;
(MMM) SEQ ID NO:388-[SEQ ID NO:389]_{(0 or 2-19)}-SEQ ID NO:390;
(NNN) SEQ ID NO:394-[SEQ ID NO:395]_{(0 or 2-19)}-SEQ ID NO:396;
(OOO) SEQ ID NO:400-[SEQ ID NO:401]_{(0 or 2-19)}-SEQ ID NO:402;
(PPP) SEQ ID NO:406-[SEQ ID NO:407]_{(0 or 2-19)}-SEQ ID NO:408; and
(QQQ) SEQ ID NO:412-[SEQ ID NO:413]_{(0 or 2-19)}-SEQ ID NO:414;
wherein the domain in brackets is an optional internal domain.

In one embodiment, the optional internal domain may be absent. In another embodiment, the optional internal domain is present in 2-19 copies, such as in 2-3 copies.

In another aspect, the invention provides polypeptides comprising or consisting of a polypeptide having at least 50% identity over its length with the amino acid sequence selected from the group consisting of SEQ ID NO: 415-497. In various further embodiments, the polypeptides comprise or consist of a polypeptide having at least 75% identity, 90% identity, or 100% identity over its length with the amino acid sequence selected from the group consisting of SEQ ID NO: 415-497.

In another embodiment, the invention provides a protein assembly comprising a plurality of polypeptides of the invention having the same amino acid sequence. In various further embodiments, the invention provides recombinant nucleic acids encoding a polypeptides of the invention, recombinant expression vectors comprising the nucleic acid of the invention operatively linked to a promoter, and recombinant host cells comprising the recombinant expression vectors of the invention.

In one aspect, a method is provided. A computing device determines a protein repeating unit. The protein repeating unit includes one or more protein helices and one or more protein loops. The computing device generates a protein backbone structure that includes at least one copy of the protein repeating unit. The computing device determines whether a distance between a pair of helices of the protein backbone structure is between a lower distance threshold and an upper distance threshold. After determining that the distance between the pair of helices of the protein backbone structure is between the lower distance threshold and the upper distance threshold, the computing device is used for: generating a plurality of protein sequences based on the protein backbone structure, selecting a particular protein sequence of the plurality of protein sequences based on an energy landscape for the particular protein sequence, where the energy landscape includes information about energy and distance from a target fold of the particular protein sequence, and generating an output based on the particular protein sequence.

In another aspect, a computing device is provided. The computing device includes one or more data processors and a computer-readable medium, configured to store at least computer-readable instructions that, when executed, cause the computing device to perform functions. The functions include: determining a protein repeating unit, where the protein repeating unit includes one or more protein helices and one or more protein loops; generating a protein backbone structure that includes at least one copy of the protein repeating unit; determining whether a distance between a pair of helices of the protein backbone structure is between a lower distance threshold and an upper distance threshold; and after determining that the distance between the pair of helices of the protein backbone structure is between the lower distance threshold and the upper distance threshold, using the computing device for: generating a plurality of protein sequences based on the protein backbone structure, selecting a particular protein sequence of the plurality of protein sequences based on an energy landscape for the particular protein sequence, where the energy landscape includes information about energy and distance from a target fold of the particular protein sequence, and generating an output based on the particular protein sequence.

In another aspect, a computer-readable medium is provided. The computer-readable medium is configured to store at least computer-readable instructions that, when executed by one or more processors of a computing device, cause the computing device to perform functions. The functions include: determining a protein repeating unit, where the protein repeating unit includes one or more protein helices and one or more protein loops; generating a protein backbone structure that includes at least one copy of the protein repeating unit; determining whether a distance between a pair of helices of the protein backbone structure is between a lower distance threshold and an upper distance threshold; and after determining that the distance between the pair of helices of the protein backbone structure is between the lower distance threshold and the upper distance threshold, using the computing device for: generating a plurality of protein sequences based on the protein backbone structure, selecting a particular protein sequence of the plurality of protein sequences based on an energy landscape for the particular protein sequence, where the energy landscape includes information about energy and distance from a target fold of the particular protein sequence, and generating an output based on the particular protein sequence.

In another aspect, a device is provided. The device comprises: means for determining a protein repeating unit, where the protein repeating unit includes one or more protein helices and one or more protein loops; means for generating a protein backbone structure that includes at least one copy of the protein repeating unit; means for determining whether a distance between a pair of helices of the protein backbone structure is between a lower distance threshold and an upper distance threshold; and means for, after determining that the distance between the pair of helices of the protein backbone structure is between the lower distance threshold and the upper distance threshold: generating a plurality of protein sequences based on the protein backbone structure, selecting a particular protein sequence of the plurality of protein sequences based on an energy landscape for the particular protein sequence, where the energy landscape includes information about energy and distance from a target fold of the particular protein sequence, and generating an output based on the particular protein sequence.

### BRIEF DESCRIPTION OF THE DRAWINGS

FIG. 1: Schematic overview of the computational design method. The lengths of each helix and loop were systematically enumerated. For each choice of (a) helix and loop lengths, individual repeat units were built up from fragments of proteins of known structure, and then propagated to generate extended (b) repeating structures with right-handed or left-handed twist.

FIG. 2: Characterization of designed repeat proteins. (a), overall summary. Values for subset with disulfide bonds are in parentheses. (b), results on six representative designs. Top row (c): design models. Second row (d): computed energy landscapes. Energy is on y axis (REU, Rosetta energy unit) and RMSD from design model on x axis. All six landscapes are strongly funneled into the designed energy minimum. Third row (e): CD spectra collected at 25° C., 95° C. and back to 25° C. The proteins do not denature within this temperature range (MRE, mean residue elipticity; deg·cm²·dmol⁻¹·residue⁻¹). Bottom row (f): SEC elution profile directly after affinity chromatography purification. The designs are mostly monodisperse. The maximum absorbance at 280 nm was normalized to 1.

FIG. 3: Crystal structures of fifteen designs are in close agreement with the design models. Insets in circles show the overall shape of the repeat protein. The RMSD values across all backbone heavy atoms are: (a) 1.50 Å (DHR4), (b) 1.73 Å (DHR5), (c) 1.30 Å (DHR7), (d) 2.28 Å (DHR8), (e) 1.79 Å (DHR10), (f) 2.38 Å (DHR14), (g) 1.21 Å (DHR18), (h) 0.87 Å (DHR49), (i) 1.33 Å (DHR53), (j) 0.93 Å (DHR54), (k) 1.54 Å (DHR64), (1) 0.67 Å (DHR71), (m) 1.73 Å (DHR76), (n) 1.04 Å (DHR79), (o) 0.65 Å (DHR81). Hydrophobic side chains in the crystal structures are largely captured by the designs (FIG. 6).

FIG. 4: Computational protocol for designing de novo repeat proteins. (a), flowchart of the design protocol. (b), low resolution backbone build. (c), quick full-atom design improves the backbone model. The superposition in the middle highlights the structural changes introduced. (d), structural profile: a 9-residue fragment is matched against the PDB repository for structures within 0.5 Å RMSD. The sequences from these structures are used to generate a sequence profile that influences design. e, packing filters were used to discard designs with cavities in the core, illustrated as spheres.

FIG. 5: Model validation by in silico folding. To assess folding robustness seven sequence variants were made for each design. (a-g) illustrate the energy landscape explored by Rosetta ab-initio. Shown are the protein models produced by ab initio search, by side chain repacking and minimization (relax). Models in deep global energy minima near the relaxed structures are considered folded. The variant with highest density of ab initio models near the relax region was chosen for experimental characterization (box). (h), Jalview sequence alignment of the first 100 residues of the variants (from top to bottom: SEQ ID NOs: 581-588). The bars indicate-sequence conservation and how often the consensus sequence occurs.

FIG. 6: Superposition between single internal repeats (second repeat) of designs and crystal structures. (a) 1.50 Å (DHR4), (b) 1.73 Å (DHR5), (c) 1.30 Å (DHR7), (d) 2.28 Å (DHR8), (e) 1.79 Å (DHR10), (f) 2.38 Å (DHR14), (g) 1.21 Å (DHR18), (h) 0.87 Å (DHR49), (i) 1.33 Å (DHR53), (j) 0.93 Å (DHR54), (k) 1.54 Å (DHR64), (1) 0.67 Å (DHR71), (m) 1.73 Å (DHR76), (n) 1.04 Å (DHR79), (o) 0.65 Å (DHR81). DHR7 and 18 show intra repeat disulphide bonds while DHR4 and 81 form inter-repeat cystines. DHR5 does not form the expected S—S bond. Core side chains in design recapitulate the conformation observed in the crystal structures. Even when the backbone is shifted (e.g. DHR5, 8, 15), rotamers are by large correctly predicted.

FIG. 7: Designs are stable to chemical denaturation by guanidine HCl (GuHCl). Circular dichroism monitored GuHCl denaturant experiments were carried for two designs for which crystal structures were solved (DHR4 and DHR14), two with overall shapes confirmed by SAXS (DHR21 and DHR62), and two with overall shapes inconsistent with SAXS (DHR17 and DHR67). In contrast to almost all native proteins, four of the six proteins do not denature at GuHCl concentrations up to 7.5 M. Both designs not confirmed by SAXS were extremely stable to GuHCl denaturation and hence are very well folded proteins; the discrepancies between the computed and experimental SAXS profiles may be due to small amounts of oligomeric species or variation in overall twist.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the invention can be used in combination, unless the context clearly dictates otherwise.

In a first aspect, the present disclosure provides polypeptides comprising or consisting of the amino acid sequence selected from the group consisting of:
(a) SEQ ID NO:1-[SEQ ID NO:2]_{(0 or 2-19)}-SEQ ID NO:3;
(b) SEQ ID NO:7-[SEQ ID NO:8]_{(0 or 2-19)}-SEQ ID NO:9;
(c) SEQ ID NO:13-[SEQ ID NO:14]_{(0 or 2-19)}-SEQ ID NO:15;
(d) SEQ ID NO:19-[SEQ ID NO:20]_{(0 or 2-19)}-SEQ ID NO:21;
(e) SEQ ID NO:25-[SEQ ID NO:26]_{(0 or 2-19)}-SEQ ID NO:27;
(f) SEQ ID NO:31-[SEQ ID NO:32]_{(0 or 2-19)}-SEQ ID NO:33;
(g) SEQ ID NO:37-[SEQ ID NO:38]_{(0 or 2-19)}-SEQ ID NO:39;
(h) SEQ ID NO:43-[SEQ ID NO:44]_{(0 or 2-19)}-SEQ ID NO:45;
(i) SEQ ID NO:49-[SEQ ID NO:50]_{(0 or 2-19)}-SEQ ID NO:51;
(j) SEQ ID NO:55-[SEQ ID NO:56]_{(0 or 2-19)}-SEQ ID NO:57;
(k) SEQ ID NO:61-[SEQ ID NO:62]_{(0 or 2-19)}-SEQ ID NO:63;
(l) SEQ ID NO:67-[SEQ ID NO:68]_{(0 or 2-19)}-SEQ ID NO:69;
(m) SEQ ID NO:73-[SEQ ID NO:74]_{(0 or 2-19)}-SEQ ID NO:75;
(n) SEQ ID NO:79-[SEQ ID NO:80]_{(0 or 2-19)}-SEQ ID NO:81;
(o) SEQ ID NO:85-[SEQ ID NO:86]_{(0 or 2-19)}-SEQ ID NO:87;
(p) SEQ ID NO:91-[SEQ ID NO:92]_{(0 or 2-19)}-SEQ ID NO:93;
(q) SEQ ID NO:97-[SEQ ID NO:98]_{(0 or 2-19)}-SEQ ID NO:99;
(r) SEQ ID NO:103-[SEQ ID NO:104]_{(0 or 2-19)}-SEQ ID NO:105;
(s) SEQ ID NO:109-[SEQ ID NO:110]_{(0 or 2-19)}-SEQ ID NO:111;
(t) SEQ ID NO:115-[SEQ ID NO:116]_{(0 or 2-19)}-SEQ ID NO:117;
(u) SEQ ID NO:121-[SEQ ID NO:122]_{(0 or 2-19)}-SEQ ID NO:123;
(v) SEQ ID NO:127-[SEQ ID NO:128]_{(0 or 2-19)}-SEQ ID NO:129;
(w) SEQ ID NO:133-[SEQ ID NO:134]_{(0 or 2-19)}-SEQ ID NO:135;
(x) SEQ ID NO:139-[SEQ ID NO:140]_{(0 or 2-19)}-SEQ ID NO:141;
(y) SEQ ID NO:145-[SEQ ID NO:146]_{(0 or 2-19)}-SEQ ID NO:147;
(z) SEQ ID NO:151-[SEQ ID NO:152]_{(0 or 2-19)}-SEQ ID NO:153;
(aa) SEQ ID NO:157-[SEQ ID NO:158]_{(0 or 2-19)}-SEQ ID NO:159;
(bb) SEQ ID NO:163-[SEQ ID NO:164]_{(0 or 2-19)}-SEQ ID NO:165;
(cc) SEQ ID NO:169-[SEQ ID NO:170]_{(0 or 2-19)}-SEQ ID NO:171;
(dd) SEQ ID NO:175-[SEQ ID NO:176]_{(0 or 2-19)}-SEQ ID NO:177;
(ee) SEQ ID NO:181-[SEQ ID NO:182]_{(0 or 2-19)}-SEQ ID NO:183;
(ff) SEQ ID NO:187-[SEQ ID NO:188]_{(0 or 2-19)}-SEQ ID NO:189;
(gg) SEQ ID NO:193-[SEQ ID NO:194]_{(0 or 2-19)}-SEQ ID NO:195;
(hh) SEQ ID NO:199-[SEQ ID NO:200]_{(0 or 2-19)}-SEQ ID NO:201;
(ii) SEQ ID NO:205-[SEQ ID NO:206]_{(0 or 2-19)}-SEQ ID NO:207;
(jj) SEQ ID NO:211-[SEQ ID NO:212]_{(0 or 2-19)}-SEQ ID NO:213;
(kk) SEQ ID NO:217-[SEQ ID NO:218]_{(0 or 2-19)}-SEQ ID NO:219;
(ll) SEQ ID NO:223-[SEQ ID NO:224]_{(0 or 2-19)}-SEQ ID NO:225;
(mm) SEQ ID NO:229-[SEQ ID NO:230]_{(0 or 2-19)}-SEQ ID NO:231;
(nn) SEQ ID NO:235-[SEQ ID NO:236]_{(0 or 2-19)}-SEQ ID NO:237;
(oo) SEQ ID NO:241-[SEQ ID NO:242]_{(0 or 2-19)}-SEQ ID NO:243;
(pp) SEQ ID NO:247-[SEQ ID NO:248]_{(0 or 2-19)}-SEQ ID NO:249;
(qq) SEQ ID NO:253-[SEQ ID NO:254]_{(0 or 2-19)}-SEQ ID NO:255;
(rr) SEQ ID NO:259-[SEQ ID NO:260]_{(0 or 2-19)}-SEQ ID NO:261;
(ss) SEQ ID NO:265-[SEQ ID NO:266]_{(0 or 2-19)}-SEQ ID NO:267;
(tt) SEQ ID NO:271-[SEQ ID NO:272]_{(0 or 2-19)}-SEQ ID NO:273;
(uu) SEQ ID NO:277-[SEQ ID NO:278]_{(0 or 2-19)}-SEQ ID NO:278;
(vv) SEQ ID NO:283-[SEQ ID NO:284]_{(0 or 2-19)}-SEQ ID NO:285;
(ww) SEQ ID NO:289-[SEQ ID NO:290]_{(0 or 2-19)}-SEQ ID NO:291;
(xx) SEQ ID NO:295-[SEQ ID NO:296]_{(0 or 2-19)}-SEQ ID NO:297;
(yy) SEQ ID NO:301-[SEQ ID NO:302]_{(0 or 2-19)}-SEQ ID NO:303;
(zz) SEQ ID NO:307-[SEQ ID NO:308]_{(0 or 2-19)}-SEQ ID NO:309;
(aaa) SEQ ID NO:313-[SEQ ID NO:314]_{(0 or 2-19)}-SEQ ID NO:315;
(bbb) SEQ ID NO:319-[SEQ ID NO:320]_{(0 or 2-19)}-SEQ ID NO:321;
(ccc) SEQ ID NO:325-[SEQ ID NO:326]_{(0 or 2-19)}-SEQ ID NO:327;
(ddd) SEQ ID NO:331-[SEQ ID NO:332]_{(0 or 2-19)}-SEQ ID NO:333;
(eee) SEQ ID NO:337-[SEQ ID NO:338]_{(0 or 2-19)}-SEQ ID NO:339;
(fff) SEQ ID NO:343-[SEQ ID NO:344]_{(0 or 2-19)}-SEQ ID NO:345;
(ggg) SEQ ID NO:349-[SEQ ID NO:350]_{(0 or 2-19)}-SEQ ID NO:351;
(hhh) SEQ ID NO:355-[SEQ ID NO:356]_{(0 or 2-19)}-SEQ ID NO:357;
(iii) SEQ ID NO:361-[SEQ ID NO:362]_{(0 or 2-19)}-SEQ ID NO:363;
(jjj) SEQ ID NO:367-[SEQ ID NO:368]_{(0 or 2-19)}-SEQ ID NO:369;
(kkk) SEQ ID NO:373-[SEQ ID NO:374]_{(0 or 2-19)}-SEQ ID NO:375;
(lll) SEQ ID NO:379-[SEQ ID NO:380]_{(0 or 2-19)}-SEQ ID NO:381;
(mmm) SEQ ID NO:385-[SEQ ID NO:386]_{(0 or 2-19)}-SEQ ID NO:387;
(nnn) SEQ ID NO:391-[SEQ ID NO:392]_{(0 or 2-19)}-SEQ ID NO:393;
(ooo) SEQ ID NO:397-[SEQ ID NO:398]_{(0 or 2-19)}-SEQ ID NO:399;
(ppp) SEQ ID NO:403-[SEQ ID NO:404]_{(0 or 2-19)}-SEQ ID NO:405; and
(qqq) SEQ ID NO:409-[SEQ ID NO:410]_{(0 or 2-19)}-SEQ ID NO:411;
wherein the domain in brackets is an optional internal domain.

The polypeptides of the invention represent novel repeat proteins with precisely specified geometries identified using the methods of the invention, opening up a wide array of new possibilities for biomolecular engineering. The polypeptides of this aspect include 2 or 3 domains, and are represented in Table 1 below, reflected in each row showing listed as “DHRx_variants” (where x is replaced by a specific number in the table). As shown in the table, the residues in brackets are possible variant positions of the residue immediately preceding it. The domains noted as “Ncap” and “Ccap” are always present, while the domain listed as “internal” is optional. When present, the “internal” domain is present in 2-19 copies

TABLE 1

| Module | Ncap | Internal | Ccap |
|---|---|---|---|
| DHR1_variants | G[SDN]C[SDT]D[E]Q[DE]V[I | C[AKN]D[QS]C[A]V[I]AK[A | R[END]D[EK]C[A]V[I]R[KED] |
| | ET]AK[RE]D[KER]AS[AYR]S | DR]AAS[ARY]S[A]II[V]R[KE | K[AN]AAS[KR]S[A]II[LE]R |
| | [KED]T[RDE]I[V]R[KE]E[NQ | A]AVI[AL]E[T]K[QE]N[LAF] | [KEN]AVQ[KER]E[DKQ]K[QE] |
| | R]V[A]I[AL]E[KQ]K[EN]N[Y | PN[G]Y[ND]S[PAE]E[DQ]V[A] | N[LAF]P[E]N[G]Y[ND]S[PE |
| | RA]PN[G]Y[ND]S[PA]E[DKT] | V[IA]AD[TEI]VAAAIV[I]K | N]E[DKN]V[A]V[KIA]E[KRN] |
| | K[TQD]V[IA]AD[KER]V[EL] | [AEL]AI[V]I[ALV]E[KD]G[SQ] | D[IKT]VK[EHR]R[KDE]AIE |
| | AAK[ER]IV[I]K[AL]K[ER]I[V] | N[AS]PN[G]G[SD] (SEQ ID | [KR]K[DEQ]AI[R]K[ERQ]E[K |
| | I[ALV]E[K]G[ERS]N[SRD]P | NO: 2) | DR]G[SAQ]N[AD]PN[G] (SEQ |
| | N[G]G[SDN] (SEQ ID NO: 1) | | ID NO: 3) |
| DHR2_design | SDADEAAKEANKAENKAR | DAVEAAKEAAKALNKALN | DAVEKAKEAAKNLNKALN |
| | NRNDDEAAKAVKLIKEAIER | RNDDEAAKAVALIAEAIIRA | RNDDEQAKHVAKQAENIIR |
| | AKKRNES (SEQ ID NO: 10) | LKRNES (SEQ ID NO: 11) | ALKRNES (SEQ ID NO: 12) |
| DHR2_variants | S[DET]D[TS]A[S]D[E]E[DKR] | D[TE]AV[IL]E[KQ]AAK[AE] | D[ES]AV[IL]E[KRD]K[RN]A |
| | AA[KRE]K[RE]E[LAR]AN[D | E[LRA]AAK[ERQ]ALN[IKQ] | K[RAQ]E[KQR]AAK[ER]N[K |
| | EQ]K[ER]AE[R]N[KE]K[LE]A | K[L]ALN[KQD]R[NQ]N[HGE] | ET]LN[IKS]K[EQR]ALN[QKD] |
| | R[E]N[KRE]R[NKQ]N[G]D[N] | D[N]D[ER]E[RD]AAK[ER]AV | R[EKN]N[GH]D[SN]D[EQ]E |
| | D[ES]E[DNS]AA[QIK]K[ER]A | A[K]L[KR]IAE[KR]AIIR[EAL] | [D]Q[EKA]AK[R]H[KEN]VA |
| | VK[E]L[K]IK[QE]E[RT]AIE[K | ALK[QER]R[QK]N[G]E[SD]S | [K]K[E]Q[ETR]AE[RK]N[QK] |
| | T]R[EQ]AK[E]K[ER]R[QK]N[G] | [DER] (SEQ ID NO: 8) | IIR[EKQ]A[D]LK[QR]R[KDE] |
| | E[SD]S[DR] (SEQ ID NO: 7) | | N[G]E[DQ]S[DET] (SEQ ID |
| | | | NO: 9) |
| DHR3_design | SSEDTVRKIAQKCSEAIRESN | SELAVRIIAQVCSEAIRESND | SELAKRIIKQVCSEAKRESN |
| | DCEEAARKCAKTISEAIRES | CECAARICAKIISEAIRESNS | DTECAKRICTKIKSEAKRES |
| | NS (SEQ ID NO: 16) | (SEQ ID NO: 17) | NS (SEQ ID NO: 18) |
| DHR3_variants | S[D]S[T]E[D]D[EQ]T[ADE]V | S[TE]E[D]LA[LT]V[I]R[K]II | S[DEP]E[D]L[K]A[LR]K[ERD] |
| | [I]R[KQ]K[ERD]I[AV]A[S]Q | [AV]A[S]Q[AE]V[A]C[AVI]S | R[KQ]II[AV]K[DEN]Q[EA]V |
| | [KE]K[DQR]C[AVI]S[AR]E[KD | [AR]E[A]AIR[KEQ]E[T]S[A]N | [A]C[EAK]S[REK]E[A]AK[R]R |
| | N]A[D]IR[KEQ]E[KT]S[ENQ] | D[N]C[T]E[DK]C[AS]AAR[K | [EKQ]E[TV]S[A]N[K]D[N]T |
| | N[K]D[N]C[T]E[DRT]E[KR]A | EH]IC[A]AK[ETR]II[V]S[RAE] | [DEK]E[DK]C[AS]AK[TDN]R |
| | AR[KQE]K[DER]C[A]AK[ET | E[AKQ]A[L]I[AV]R[EK]E[Q | [KE]IC[AST]T[KEQ]K[QRE]IK |
| | [LT]I[AT]R[KET]E[KQ]S[AL] | R]S[AQ]N[G]S[D] (SEQ ID | [RE]S[ERK]E[AQR]A[L]K[RE] |
| | N[G]S[N] (SEQ ID NO: 13) | NO: 14) | R[EKN]E[Q]S[NQ]N[G]S[D] |
| | | | (SEQ ID NO: 15) |
| DHR6_design | SEEKEEALKKVREAAKKLG | AYEAAEALFKVLEAAYKLG | AYEAAERLFEELERAYEEGS |
| | SSDEEARKCFEEAREWAER | SSAEEACECFNQAAEWAER | SAEEACEEFNKKEEEAHRK |
| | TGSS (SEQ ID NO: 22) | TGSG (SEQ ID NO: 23) | GKK (SEQ ID NO: 24) |
| DHR6_variants | S[D]E[D]E[KD]K[DER]E[KN | AY[AW]E[LQR]AAE[HK]AL | AY[AK]E[DQ]AAE[HKR]R[E |
| | Q]E[TKR]AL[EKR]K[EQN]K | [A]F[A]K[EQN]VL[A]E[K]AA | K]L[A]F[A]E[QKR]E[VQ]L[A] |
| | [ELQ]VR[E]E[DRT]AAKK[EQ] | Y[HAW]K[R]L[N]GS[A]SAE | ER[EKN]AY[WAH]E[K]E[RN |
| | L[NQ]GS[A]S[N]D[ESQ]E[D] | [DR]E[Q]AC[ARL]E[KQ]C[A | Q]GS[KLE]S[D]AE[RDK]E[Q |
| | E[QDH]AR[EDK]K[ERQ]C[A | W]FN[DES]Q[ER]AAE[QKR] | R]AC[ART]E[KR]E[Q]F[Y]N |
| | NW]F[TW]E[RK]E[RQ]AR[A | WAE[KQS]R[EK]T[N]GS[AV] | [DS]K[RE]K[ERD]E[AQ]E[KR] |
| | KS]E[KNQ]W[A]AE[KNS]R[E | G[NT] (SEQ ID NO: 20) | E[KR]AH[KQR]R[KE]K[END] |
| | Q]T[A]GS[AV]S[NDT] (SEQ | | GK[QT]K[NDT] (SEQ ID NO: |
| | ID NO: 19) | | 21) |
| DHR7_design | STKEDARSTCEKAARKAAE | TKEAARSFCEAAARAAAES | TKEAARSFCEAAKRAAKES |
| | SNDEEVAKQAAKDCLEVAK | NDEEVAKIAAKACLEVAKQ | NDEEVEKIAKKACKEVAKQ |
| | QAGMP (SEQ ID NO: 28) | AGMP (SEQ ID NO: 29) | AGMP (SEQ ID NO: 30) |
| DHR7_variants | ST[SD]K[QE]E[DR]D[K]AR[K | T[RAE]K[R]E[KR]AAR[KEQ] | T[RKP]K[QR]E[K]AAR[KE]S |
| | ET]S[EKR]T[EQ]CE[RKQ]K | S[EDK]FCE[KQR]AAAR[EK] | [ERA]FCE[KR]AAK[E]R[KEQ] |
| | [RQ]AAR[EQ]K[REH]AAE[KN | AAAE[R]S[QEH]N[KR]D[S]E | AAK[RDE]E[K]S[QKN]N[GK] |
| | R]S[QKD]N[KR]D[NS]E[PK]E | [PKT]E[TKD]V[A]AK[ER]I[V | D[S]E[PDS]E[KQT]V[A]E[KR] |
| | [DNK]V[EDQ]AK[ERH]Q[KR | A]AAK[RYI]ACL[AKR]E[AQ | K[ER]I[VA]AK[RED]K[ERQ] |
| | E]AAK[REQ]D[ERK]CL[AKR] | R]V[A]AK[DEQ]Q[EN]AGM | ACK[ERQ]E[QAK]V[A]A[KL |
| | E[RK]V[A]AK[DQE]Q[KRE] | [AL]P[DT] (SEQ ID NO: 26) | R]K[ERD]Q[E]AGM[AL]P[DT] |
| | AGM[AL]P[DTN] (SEQ ID | | (SEQ ID NO: 27) |
| | NO: 25) | | |
| DHR8_design | SDEMKKVMEALKKAVELA | DEMAKVMLALAKAVLLAA | DEMAKKMLELAKRVLDAA |
| | KKNNDDEVAREIERAAKEIV | KNNDDEVAREIARAAAEIVE | KNNDDETAREIARQAAEEV |
| | EALRENNS (SEQ ID NO: 34) | ALRENNS (SEQ ID NO: 35) | EADRENNS (SEQ ID NO: 36) |
| DHR8_variants | S[DT]D[STN]E[KDT]M[AIQ] | D[ESK]E[DKL]M[AV]A[WIL] | D[ER]E[DKQ]M[AV]A[WIL]K |
| | K[EQR]K[EQR]V[A]M[KLR]E | K[ER]V[A]M[AL]L[AEY]A[L] | [DER]K[TED]M[AL]L[RAE]E |
| | [K]A[L]L[W]K[ERD]K[RE]AV | L[W]AK[ELQ]AV[AI]L[AR]L | [KR]L[ERW]AK[EQ]R[KES]V |
| | [AI]E[QDK]L[QI]AK[SR]K[N | [IQE]AAK[QER]N[SD]N[G]D | [AI]L[AR]D[RKQ]A[L]AK[QR] |
| | QD]N[SD]N[G]D[N]D[EPK]E | [N]D[A]E[DK]V[AQ]AR[AIQ] | N[SDE]N[G]D[N]D[A]E[KD]T |
| | [DK]V[AQ]AR[KE]E[RKA]IE | E[RQI]IAR[KEH]AAA[EK]E | [KES]AR[AIK]E[KR]I[QRT]A |
| | [KQR]R[KEH]AAK[DEQ]E[R]I | [RQ]I[A]V[A]E[RDK]AL[A]R | R[EKD]Q[KEN]AA[EV]E[RK] |
| | [A]V[KAE]E[KDR]AL[A]R[K | [AEK]E[KQT]N[VAI]N[TQK]S | E[ADK]V[A]E[RKD]A[KNE]D |
| | EN]E[KNQ]N[VAI]N[DPT]S | [DT] (SEQ ID NO: 32) | [LAE]R[AKD]E[KRQ]N[G]N |
| | [DQT] (SEQ ID NO: 31) | | [QTE]S[DT] (SEQ ID NO: 33) |
| DHR9_design | SYEDEAEEKARRVAEKVER | YEVIAEIVARIVAEIVEALKR | YEVIKEIVQRIVEEIVEALKR |
| | LKRSGTSEDEIAEEVAREISE | SGTSEDEIAEIVARVISEVIRT | SGTSEDEINEIVRRVKSEVER |
| | VIRTLKESGSS (SEQ ID NO: | LKESGSS (SEQ ID NO: 41) | TLKESGSS (SEQ ID NO: 42) |
| | 40) | | |
| DHR9_variants | S[D]Y[STD]E[DT]D[E]E[DT] | Y[ESD]E[DKS]V[AED]IAE[K | Y[SDE]E[DR]V[EQT]IK[RDQ] |
| | AE[KR]E[RK]K[RDE]AR[EK] | HR]I[V]V[IL]AR[QEA]I[AV]V | E[KH]I[V]V[IL]Q[RET]R[EQA] |
| | R[KT]V[I]AE[NRD]K[DET]V | [I]AE[AKR]I[V]V[A]E[KQR]A | I[AV]V[IAK]E[RKN]E[AKR]I |
| | [A]E[KR]R[KE]LK[YWA]R[KE | LK[QWH]R[EDQ]S[NE]GT[V] | [V]V[EIK]E[KR]ALK[QER]R |
| | D]S[NKD]GT[V]S[D]E[PNT]D | S[D]E[PT]D[EQT]E[LQ]IAE[K | [KE]S[NET]GT[V]S[D]E[PS]D |
| | [ET]E[KQ]IAE[KDQ]E[KRT]V | RD]I[V]V[A]AR[EHI]V[I]I[VL] | [E]E[QLK]IN[KRE]E[KR]I[V]V |
| | [A]AR[EKD]E[QDN]I[VL]S[A | S[AEK]E[RV]V[I]I[L]R[EKQ] | [ESA]R[KQ]R[IHQ]V[I]K[QR |
| | RE]E[KR]V[TDK]I[AL]R[KEQ] | T[AEQ]LK[EQT]E[NKR]S[DQ | E]S[EDK]E[KRV]V[IAT]E[KR] |
| | T[EDK]LK[EQ]E[KRD]S[RD | N]GS[KQ]S (SEQ ID NO: 38) | R[KE]T[AEQ]L[QKN]K[REN] |
| | K]GS[KQ]S[D] (SEQ ID | | E[KRD]S[QDN]GS[KQE]S[D |
| | NO: 37) | | NP] (SEQ ID NO: 39) |
| DHR10_design | SSEKEELRERLVKIVVENAK | SSEVLELAIRLIKEVVENAQ | SSETLKRAIEEIRKRVEEAQR |
| | RKGDDTEEAREAAREAFEL | REGYDISEAARAAAEAFKR | EGNDISEAARQAAEEFRKK |
| | VREAAERAGID (SEQ ID NO: | VAEAAKRAGIT (SEQ ID NO: | AEELKRRGD (SEQ ID NO: |
| | 46) | 47) | 48) |
| DHR10_variants | S[T]S[DE]E[DKT]K[AS]E[K]E | S[T]S[KNT]E[DTK]V[A]L[IA | S[T]S[TKW]E[DKS]T[ADR]L |
| | [KNR]L[IT]R[AKQ]E[KRN]R | V]E[KQ]L[IT]A[V]I[A]R[KE]L | [IAV]K[ER]R[EKD]A[V]I[A]E |
| | [KE]L[I]V[I]KI[KT]VV[AK]E | [I]I[V]KE[IK]VV[A]EN[AL]A | [KD]E[HKD]I[V]R[K]K[EQR]R |
| | [K]N[AL]AK[QER]R[KE]K[QN | Q[AW]RE[QKN]GY[EQ]D[N]I | [E]V[A]E[KQ]E[KRT]AQ[AL] |
| | R]GD[EQW]D[N]T[EKD]E[SD] | [V]S[AT]E[KD]AAR[QEK]A | R[KDE]E[KQ]GN[ERQ]D[NT] |
| | E[KDT]AR[AKE]E[KRD]A[D] | [D]AAE[DR]AF[VAW]K[EAQ] | I[V]S[AT]E[DQK]AAR[EKQ] |
| | AR[KE]E[K]AF[VWA]E[KR] | R[IQ]V[IA]AE[QR]AA[L]K[E | Q[ERD]AAE[KR]E[KRQ]F[V |
| | L[RI]V[IA]R[EKQ]E[RDK]AA | H]R[EHK]AGI[LD]T[VDK] | AW]R[KEA]K[RE]K[EDR]AE |
| | [L]E[KRD]R[EKD]A[S]GI[L]D | (SEQ ID NO: 44) | [QK]E[KRN]L[RA]K[HER]R[K |
| | (SEQ ID NO: 43) | | EQ]R[KE]GD[NQK] (SEQ ID |
| | | | NO: 45) |
| DHR12_design | DDEEQCREIAEKAKQTYTD | DEEICRCIAEAAKQTYTDDE | DEEIERCIEEAAKQTYTDDE |
| | DEEIARIIAEAARQTTTD | EIARIIAYAARQTTTD (SEQ | EIERIKEYARRQTTTD (SEQ |
| | (SEQ ID NO: 52) | ID NO: 53) | ID NO: 54) |
| DHR12_variants | D[N]D[ST]E[TDQ]E[D]Q[KET] | D[PK]E[TD]E[R]IC[A]R[KE]C | D[PES]E[DKN]EIE[RKD]R[K] |
| | C[A]R[KI]E[K]IAE[KR]K[QE | [LI]IAE[IR]AAK[RQ]Q[ER]T | C[LI]IE[K]E[IQ]AAK[R]Q[KE] |
| | N]AK[RQ]Q[KR]T[KDR]Y[SA | Y[ASR]T[DES]D[NTS]D[PKE] | TY[SAR]T[SD]D[TNS]D[PEQ] |
| | R]T[SD]D[TN]D[PKE]E[DKQ] | E[QDT]E[DKN]IAR[AK]I[LV] | E[DN]E[DKN]IE[KRD]R[KE |
| | E[KQA]IAR[KAE]I[ELY]IAE | IAY[AEI]AAR[KHQ]Q[KR]T | Q]I[LV]K[I]E[KD]Y[IEA]AR |
| | [KR]A[E]AR[KHQ]Q[KR]T[EQ | [Q]TTD[N] (SEQ ID NO: 50) | [EKD]R[KE]Q[EKR]T[QS]TTD |
| | R]TTD[N] (SEQ ID NO: 49) | | [N] (SEQ ID NO: 51) |
| DHR13_design | NAEDKAREVLKELKDEGSP | AEDAARAVLKALKDEGSPE | EEDASRAVLKALKDEGSPEE |
| | EEEAARQVLKDLNREGSN | EEAARAVLKALNREGSN | EARRAVEKALNREGSN |
| | (SEQ ID NO: 58) | (SEQ ID NO: 59) | (SEQ ID NO: 60) |
| DHR13_variants | N[SD]A[SDT]E[TAS]D[EK]K | A[RTE]E[TIS]D[EQK]AAR[A | E[TSK]E[DST]D[EKQ]AS[AK] |
| | [EDN]AR[ALY]E[K]V[EKQ]L | LY]A[IL]VLK[ERV]ALK[QR | R[KE]A[IK]VL[EW]K[RQE]A |
| | K[EQR]E[TKQ]LK[EQ]D[KR | N]D[QRK]E[QSR]GS[TVH]P | LK[EQR]D[QNE]E[SHQ]GS[V |
| | N]E[KQD]GS[TVL]P[SD]E[PT | [DS]E[PT]E[KST]E[Q]AAR[AL] | TK]P[SD]E[PR]E[D]E[KR]AR |
| | R]E[TRS]E[K]AAR[AEL]Q[K | A[ILQ]V[L]LK[EQR]ALN[EK] | [KN]R[EK]A[ILQ]V[A]E[KDR] |
| | EN]V[L]LK[EQR]D[EKQ]LN | R[NEQ]E[TNQ]GS[V]N[DS] | K[RED]AL[QE]N[KER]R[KN |
| | [EK]R[NKE]E[KRQ]GS[V]N | (SEQ ID NO: 56) | Q]E[TNH]GS[KQH]N[DR] |
| | [SD] (SEQ ID NO: 55) | | (SEQ ID NO: 57) |
| DHR14_design | DSEEVNERVKQLAEKAKEA | SELVNEIVKQLAEVAKEATD | SELVNEIVKQLEEVAKEATD |
| | TDKEEVIEIVKELAELAKQS | KELVIYIVKILAELAKQSTD | KELVEHIEKILEELKKQSTD |
| | TD (SEQ ID NO: 64) | (SEQ ID NO: 65) | (SEQ ID NO: 66) |
| DHR14_variants | D[NST]S[DTN]E[D]E[D]V[IE] | S[DEN]E[DKN]L[A]V[I]N[KR | S[DEP]E[DKR]L[A]V[IQ]N[K |
| | N[RKE]E[KDN]R[KEN]V[I]K | L]E[KQ]I[A]V[I]K[REQ]Q[LA] | QE]E[RDH]I[A]V[IE]K[EQ]Q |
| | [ERD]Q[KER]L[KR]AE[K]K[E | L[V]AEVAK[R]E[Q]ATD[NS] | [LAE]L[V]E[QKR]E[KR]VA[K |
| | R]AK[Q]E[KR]ATD[NS]K[RT | K[REP]E[DRS]LV[I]I[REH]Y | QR]K[DE]E[Q]ATD[NS]K[DE |
| | P]E[DSK]E[KL]V[I]I[KRE]E | [ERK]I[L]V[AL]K[RDE]I[AL] | P]E[DKN]LV[QIR]E[KR]H[EQ |
| | [KR]I[L]V[AL]K[ER]E[KT]L | L[I]A[ER]E[KQN]LAK[ER]Q | R]I[L]E[NQ]K[ER]I[AL]L[IR] |
| | [I]A[RQ]E[KNR]L[ER]AK[QS | [KDE]S[A]T[QNS]D[NST] | E[KR]E[KNQ]LK[Q]K[R]Q[R |
| | E]Q[KR]S[A]T[SNQ]D[NST] | (SEQ ID NO: 62) | SE]S[ALR]T[NQ]D[KNS] |
| | (SEQ ID NO: 61) | | (SEQ ID NO: 63) |
| DHR15_design | NDERQKQREEVRKLAEELA | DELIKQILEVAKLAFELASK | DEEIKQILETAKEAFERASK |
| | SKATDEELIKEIKKCAQLAE | ATDEELIKEILKCCQLAFELA | ATDEEEIKEILKKCQEKFEK |
| | ELASRSTN (SEQ ID NO: 70) | SRSTN (SEQ ID NO: 71) | KSRSTN (SEQ ID NO: 72) |
| DHR15_variants | N[DS]D[S]E[D]R[ETN]Q[KED] | D[P]E[TR]L[I]IK[RN]Q[LEA]I | D[P]E[DKN]E[DK]IK[RAI]Q |
| | K[RE]Q[L]R[EKQ]E[KQR]E | [A]LE[IK]V[A]AK[IL]LAF[A | [REK]I[A]LE[KQR]T[EIK]AK[I |
| | [KIR]V[A]R[E]K[DE]LA[W]E | N]E[K]LAS[QR]K[NER]A[L]T | L]E[RK]AF[AN]E[KQ]R[KDE] |
| | [KR]E[KRD]LAS[KNQ]K[NQR] | DE[P]E[NR]L[A]I[A]K[E]E[L | AS[EKQ]K[NRD]A[LI]T[DE] |
| | A[L]T[EN]D[NS]E[DSP]E[DQ] | Q]I[A]LK[ER]C[A]C[A]Q[KS] | D[ST]E[DPS]E[NKD]E[K]I[A |
| | L[A]I[RA]K[DEQ]E[QLR]I[A] | L[E]A[W]F[A]E[K]LASR[K]S | R]K[ES]E[KR]I[A]LK[ER]K[E |
| | K[Q]K[ER]C[A]AQ[KE]L[RK | [A]TN[D] (SEQ ID NO: 68) | R]C[A]Q[E]E[RKQ]K[REN]F |
| | E]A[W]E[KNQ]E[KDQ]LAS[K | | [A]E[KR]K[DER]K[DNS]S[N]R |
| | NE]R[KQD]S[A]TN[DS] (SEQ | | [KQD]S[KN]TN[DS] (SEQ ID |
| | ID NO: 67) | | NO: 69) |
| DHR16_design | NDKAKEAEELLRKALEKAE | DKAIEAVELLAKALEKALK | DKAIEEVERLAKELEKALKE |
| | KENDETAIRCVELLKEALER | ENDETAIRCVCLLAEALLRA | NDETKIREVCERAEELLRRL |
| | AKKNNN (SEQ ID NO: 76) | LKNNN (SEQ ID NO: 77) | KNNN (SEQ ID NO: 78) |
| DHR16_variants | N[D]D[T]K[T]A[S]K[DE]E[RD | D[EK]K[ET]AIEAVE[YKR]L | D[E]K[DSE]AIE[R]E[TNK]VE |
| | K]AE[KQ]E[KD]L[EKN]LR[K | [RK]LAK[ED]ALE[RLK]K[IR] | [RAL]R[KE]L[W]AK[ERD]E |
| | DE]K[EDR]AL[EK]E[RKQ]K | ALK[ERN]E[QR]NDE[KS]T[K | [KDN]LE[AKL]K[RED]ALK[E |
| | [IER]AE[QR]K[ER]E[QKR]N | D]AI[V]R[EK]C[A]VC[AL]LL | RN]E[KNQ]N[G]D[N]E[S]T[D |
| | [G]D[S]E[DKS]T[KDQ]AI[LQ] | AE[R]ALL[EK]R[EL]ALK[R] | K]K[AQS]I[V]R[EK]EVC[AL |
| | R[KE]C[A]VE[K]L[K]LK[RQE] | N[QER]N[G]N[D] (SEQ ID | R]E[KR]RAE[KR]E[KQR]LL |
| | E[KQ]ALE[KR]R[EIL]AK[ER] | NO: 74) | [AEK]R[ED]R[AD]LK[RE]N[K |
| | K[ER]N[QRD]N[G]N[D] | | Q]N[G]N[QK] (SEQ ID NO: |
| | (SEQ ID NO: 73) | | 75) |
| DHR17_design | SSEDAREKIEQLCREAKEIAE | SEVAREAIECLCRIAKLIAEL | SEVAREAIECLSRIAKLIEEL |
| | RAKQQNSQEEAREAIEKLLR | AKQANSQEVAREAIEALLRI | AKQANSQEVKREAQEALDR |
| | IAKRIAELAKQANQ (SEQ ID | AKLIAELAKQANQ (SEQ ID | IQKLIEELQKQANQ (SEQ ID |
| | NO: 82) | NO: 83) | NO: 84) |
| DHR17_variants | S[ND]SE[DT]D[EQ]A[N]R[KE | S[AP]E[DK]VIA]AR[ALQ]E[R | S[PAR]E[DKS]V[A]AR[KTE] |
| | L]E[KR]K[NDR]IE[KD]Q[KE] | DK]AIE[KR]C[A]LC[LAE]R[E | E[QK]AI[K]E[KR]C[A]LS[KQ |
| | LC[LRA]R[KEQ]E[KQR]AK[E | KH]I[V]AK[RE]LIAELAK[QE | N]R[EKT]I[V]A[KE]K[QRE]LI |
| | Q]E[KR]I[EV]AE[RN]R[EKT] | R]Q[EN]AN[G]S[D]Q[K]E[DK | E[KQR]E[RD]LAK[ERN]Q[E] |
| | AK[N]Q[RKE]Q[SEN]N[GK]S | T]V[A]AR[E]E[RVK]AI[V]E | AN[GK]S[D]Q[DE]E[DKT]V |
| | [N]Q[KR]E[D]E[DQS]AR[IKL] | [KDQ]ALL[AR]R[KET]I[V]AK | [A]K[RA]R[TKE]E[KIQ]AQ[K |
| | E[RK]AI[V]E[KRS]K[ERQ]L | [EQ]LIAE[RK]LAK[Q]Q[DKR] | E]E[K]AL[AKN]D[EKQ]R[KE |
| | L[AR]R[KE]I[V]AK[EQR]R[K | AN[GK]Q[TS] (SEQ ID NO: | Q]I[V]Q[DER]K[Q]LI[Q]E[KR] |
| | NQ]IAE[KR]L[E]AK[QRE]Q | 80) | E[KQ]LQ[KER]K[R]Q[DEN] |
| | [KRE]AN[GK]Q[TS] (SEQ ID | | AN[GK]Q[ETS] (SEQ ID NO: |
| | NO: 79) | | 81) |
| DHR18_design | DIEKLCKKAESEAREARSKA | DIAKLCIKAASEAAEAASKA | DIAKKCIKAASEAAEEASKA |
| | EELRQRHPDSQAARDAQKL | AELAQRHPDSQAARDAIKL | AEEAQRHPDSQKARDEIKE |
| | ASQAEEAVKLACELAQEHP | ASQAAEAVKLACELAQEHP | ASQKAEEVKERCERAQEHP |
| | NA (SEQ ID NO: 88) | NA (SEQ ID NO: 89) | NA (SEQ ID NO: 90) |
| DHR18_variants | D[STN]I[AW]E[D]K[D]L[ER] | D[EQ]I[A]AK[LQR]L[RK]CI | D[EKQ]I[AEQ]AK[RI]K[RED] |
| | CK[EQR]K[ETH]AE[QKR]S[K | [L]K[ET]AAS[AIQ]E[LAR]AA | CI[L]K[ER]A[DKE]AS[IAE]E |
| | EN]E[LA]AR[DKQ]E[KRQ]A | E[KRI]AAS[AKI]K[LAQ]AA[I] | [KR]AAE[KR]E[ANQ]AS[AIE] |
| | R[KE]S[KED]K[LRE]AE[QDK] | E[KDS]L[A]A[L]Q[KLR]R[D | K[RE]AA[I]E[QDR]E[ILK]A[L] |
| | E[KRS]L[A]R[YKE]Q[KDN] | QE]H[RAL]PD[N]S[NT]Q[ED | Q[KRS]R[KDE]H[RY]PD[NG] |
| | R[QDE]H[RAK]PD[NG]S[NT] | K]A[V]AR[KAE]D[LEK]AI[L] | S[DT]Q[EDS]K[DER]AR[KE |
| | Q[DE]A[V]AR[KNQ]D[LET]A | K[ERQ]L[AV]A[V]S[AIR]Q[A | Q]D[KER]E[AKD]I[L]K[EDR] |
| | Q[ERI]K[E]L[AV]A[V]S[EKR] | LE]AAE[KQR]AVK[YLQ]L[E | E[KRQ]A[V]S[RAI]Q[EKR]K |
| | Q[AEL]AE[KQI]E[RKQ]AVK | KQ]ACE[KRQ]LAQ[E]E[KQR] | [DLT]AE[RDK]E[KRD]VK[LA |
| | [ER]L[EKQ]ACE[KNR]LAQ[K | H[Y]PN[G]A[S] (SEQ ID NO: | I]E[RKQ]R[KDE]CE[KR]R[K] |
| | N]E[KQR]H[Y]P[K]N[G]A[S] | 86) | AQ[ED]E[KQ]H[NY]PN[G]A |
| | (SEQ ID NO: 85) | | [SQ] (SEQ ID NO: 87) |
| DHR19_design | DEIEKVREEAEKLKKKTDDE | DEILKVIKEALKLAKKTTDK | EEILKEIKEALKKAKETTDT |
| | DVLEVAREAIRAAKEATS | DVLEVAREAIRAAEEATD | EELEKAREQIRKAEESTD |
| | (SEQ ID NO: 94) | (SEQ ID NO: 95) | (SEQ ID NO: 96) |
| DHR19_variants | D[TS]E[DKN]I[KQ]E[KQD]K | D[SEQ]E[DKN]ILK[ERT]V[A] | E[DSK]E[DS]ILK[EQ]E[RKL]I |
| | [EHQ]V[A]R[IK]E[KDN]E[DR] | IK[EQR]E[Q]ALK[R]L[IV]AK | K[QEN]E[RKN]ALKK[IRE]A |
| | AE[KQN]K[ER]L[IV]K[SRA] | [QSE]K[QST]TTD[T]K[TED]D | K[QS]E[TKQ]TTD[T]T[EKS]E |
| | K[RDE]K[QT]TD[NT]D[T]E[Q | [EN]V[A]LE[KR]VAR[ELQ]E | [D]E[VD]LE[KRN]K[ER]AR[E |
| | D]D[EN]V[A]L[QKR]E[RKD] | [QKL]AIR[EK]AAE[RT]E[ND | KL]E[K]Q[TED]IR[EKQ]K[D |
| | VAR[KDE]E[LAK]AI[K]R[EK] | K]ATD[S] (SEQ ID NO: 92) | QR]AE[RT]E[KNQ]S[EKQ]TD |
| | AAK[ED]E[NDK]ATS (SEQ | | [N] (SEQ ID NO: 93) |
| | ID NO: 91) | | |
| DHR20_design | SDIEEIRQLAEELRKKSDNEE | SDVLEIVKDALELAKQSTNE | EEVLEEVKEALRRAKESTDE |
| | VRKLAQEAAELAKRSTD | EVIKLALKAAVLAAKSTD | EEIKEELRKAVEEAESTD |
| | (SEQ ID NO: 100) | (SEQ ID NO: 101) | (SEQ ID NO: 102) |
| DHR20_variants | S[TDN]D[TQ]I[VAR]E[KD]E | S[KEP]D[TKQ]V[A]L[W]E[K | E[KPS]E[DKT]V[A]L[W]E[K |
| | [KR]IR[EIQ]Q[EKR]L[TEK]AE | R]IVK[EQR]D[LKR]ALE[KR] | N]E[TIR]VK[ERA]E[KR]ALR |
| | [RKQ]E[RQD]L[VI]R[ASK]K | L[VI]AK[EQ]Q[KRD]S[AT]T | [EQ]R[KDE]AK[EQR]E[KR]S |
| | [NRT]K[EDN]S[ALT]D[T]N[D] | N[D]E[DPN]E[DK]V[AI]IK[R | [AKN]TD[N]E[DNP]E[DQR]E |
| | E[DPK]E[TDQ]V[AI]R[IQ]K | A]LALK[ELR]AAVLAAK[QR] | [KDN]IK[RAE]E[RKQ]E[ADL] |
| | [RED]LAQ[ERK]E[RTL]AAE | S[AEN]T[R]D[TS] (SEQ ID | LR[EK]K[NQR]AVE[RD]E[D |
| | [K]LAK[HQ]R[K]S[ANT]T[R] | NO: 98) | QA]AE[KQ]S[KRT]T[NR]D[T |
| | D[TS] (SEQ ID NO: 97) | | N] (SEQ ID NO: 99) |
| DHR21_design | SEKEKVEELAQRIREQLPDT | SEALKVVYLALRIVQQLPDT | QEALKSVYEALQRVQDKPN |
| | ELAREAQELADEARKSDD | ELAREALELAKEAVKSTD | TEEARESLERAKEDVKSTD |
| | (SEQ ID NO: 106) | (SEQ ID NO: 107) | (SEQ ID NO: 108) |
| DHR21_variants | S[DTN]E[KDL]K[AQS]E[K]K | S[EQD]E[KNQ]AL[W]K[E]VV | Q[EDK]E[DKR]AL[W]K[ED]S |
| | [EDR]VE[R]E[KQS]LAQ[REK] | [A]Y[KAE]LALR[QAE]I[V]V | [IKD]V[A]Y[KAE]E[KQR]AL |
| | R[KDE]I[V]R[AK]E[KN]Q[NT] | [A]Q[EKL]Q[RT]LPD[N]TE[D | Q[EKR]R[ITD]V[A]Q[EKL]D |
| | LP[K]D[N]TE[DRS]L[I]AR[E | Q]L[I]AR[KE]E[KLD]ALE[KR | [KQR]K[YHR]PNTE[D]E[DK] |
| | K]E[LKQ]AQ[ENL]E[KRQ]L | D]L[V]AK[EQR]E[KDN]AV[I] | AR[KEQ]E[KQR]S[A]LE[DQR] |
| | [V]AD[EKR]E[KDQ]AR[KEQ] | K[ER]ST[Q]D[SN] (SEQ ID | R[KEQ]AK[EQR]E[K]D[EKA] |
| | K[ERT]SD[NTR]D[SN] (SEQ | NO: 104) | V[IA]K[ET]S[R]T[NQ]D[NST] |
| | ID NO: 103) | | (SEQ ID NO: 105) |
| DHR22_design | DDAEELRERARDLLRKNGS | DDAVKLAVKAAALLAENGS | EEEVKDAVREAAELAERGS |
| | SEEEIKKVDEELEKIVRKAD | SAEEIVKVLEELLKIVEKAD | SAEEIRKQLKDRLRKVEESD |
| | S (SEQ ID NO: 112) | S (SEQ ID NO: 113) | S (SEQ ID NO: 114) |
| DHR22_variants | D[S]D[TK]AE[D]E[KT]LR[A] | D[SW]D[KET]AV[A]K[ITA]L | E[SW]E[DKS]E[QT]V[A]K[IT |
| | E[QK]R[KL]AR[A]D[KQE]LL | AV[A]K[L]AAALLAE[QKR]N | A]D[REK]AV[A]R[KEL]E[TD |
| | R[KQ]K[DEQ]NGS[AQ]S[D]E | GS[AQ]SAE[DQS]E[Q]IV[RA | K]AAE[DQ]L[QER]AE[QKR] |
| | [DKP]E[DS]E[QS]IK[N]K[RQ] | Y]K[R]VLE[H]E[ALW]L[I]L | R[KDE]GS[RE]SAE[DRS]E[R] |
| | VD[LT]E[K]E[ADL]L[I]E[KQ | [A]K[R]I[A]V[I]E[QK]K[Q]AD | IR[AY]K[E]Q[TES]LK[EHR]D |
| | R]K[RQ]I[A]V[RKI]R[DEK]K | [Q]S (SEQ ID NO: 110) | [EKN]R[LIQ]L[AE]R[KEQ]K |
| | [QDN]AD[QK]S (SEQ ID NO: | | [D]V[ILT]E[QKR]E[KNQ]S[A] |
| | 109) | | D[QT]S[D] (SEQ ID NO: 111) |
| DHR23_design | SDSEKLAKRVLKELKRRGTS | SDAMRLALRVVLELVRRGT | DDQMREALRQVLEEVRKGT |
| | DEELERMKRELEKIIKSATS | SSEILEKMMRMLIKIIQSATS | SSEQLERSMRKLIKEIKKRTS |
| | (SEQ ID NO: 118) | (SEQ ID NO: 119) | (SEQ ID NO: 120) |
| DHR23_variants | S[TDN]D[TR]S[AQ]E[DK]K[E | S[DE]D[TEK]AM[A]R[KEA]L | D[ES]D[ET]Q[EAL]M[A]R[K |
| | QR]LAK[QRD]R[EKT]V[AI]L | ALR[EK]V[AI]V[LI]LE[RQ]L | AE]E[RKQ]ALR[KE]Q[ETD]V |
| | [VR]K[ENR]E[QDL]L[A]K[R] | [A]V[AI]R[KE]R[KN]GT[EKQ] | [LI]LE[DRK]E[ADR]V[AI]R |
| | R[KN]R[NKS]GT[QE]S[D]D[S | SS[AIQ]E[DRT]I[EAN]L[I]E | [KEQ]K[ETD]GT[KQR]S[D]S |
| | P]E[DT]E[DAI]L[EI]E[KNR]R | [DKS]K[RT]M[ALI]M[A]R[EK] | [AIQ]E[DQR]Q[EDS]L[I]E[KD |
| | [K]M[ALI]K[ER]R[EQK]E[LAQ] | M[LAQ]L[I]I[QR]K[ERQ]I[V | R]R[KEQ]S[TLE]M[A]R[EQ]K |
| | L[I]E[KQR]K[RDE]I[VL]I[R | L]IQ[EK]S[EQA]AT[QK]S[T] | [EQ]L[I]I[KQ]K[RE]E[K]IK[Q |
| | KQ]K[DER]S[EQT]AT[Q]S[T] | (SEQ ID NO: 116) | R]K[NDQ]R[S]T[Q]S[DT] |
| | (SEQ ID NO: 115) | | (SEQ ID NO: 117) |
| DHR24_design | SEAEELARRAAKEAKELCK | SEAAKLALKAALEAIELCKQ | SEEAKRALKEAKELIEQCKE |
| | RSTDEELCKELKKLAELLKE | STDEELCEELVKLAQKLIEL | STDEDECRELVKRAEELIRE |
| | LAERYPD (SEQ ID NO: 124) | AKRYPD (SEQ ID NO: 125) | AKENPD (SEQ ID NO: 126) |
| DHR24_variants | SE[DQR]AE[KQ]E[KQR]L[E] | SE[RTD]AAK[ERQ]LALK[RE | SE[D]E[ANQ]AK[ERQ]R[EK] |
| | AR[E]R[EK]AA[EK]K[E]E[RK | S]AAL[AK]E[AKR]AI[L]E[KR | ALK[ER]E[NRK]AK[AEL]E[K |
| | A]AK[REQ]E[KQS]L[AV]CK | H]L[AV]CK[REQ]Q[EKD]S[Q | RN]L[A]I[L]E[RK]Q[EKR]CK |
| | R[DKE]S[KTQ]T[NR]D[N]E[D] | T]T[N]D[N]E[DNS]E[DKN]LC | [RQE]E[KQR]S[DK]T[D]D[N]E |
| | E[DKR]L[T]CK[E]E[DKL]LK | E[RQ]E[KL]LV[A]K[ER]LAQ | [DTS]D[EKQ]E[KR]CR[EKQ] |
| | [EQ]K[ER]LAE[KQR]L[EKQ] | [KES]K[ELQ]LI[VA]E[KR]LA | E[KR]LV[A]K[ER]R[KEQ]AE |
| | LK[EN]E[KQR]LAE[KRD]R | K[EQD]R[EK]Y[L]P[S]D (SEQ | [KQ]E[KR]L[EDK]I[VA]R[KE] |
| | [KEN]Y[L]PD (SEQ ID NO: | ID NO: 122) | E[KR]AK[EQR]E[KD]N[DH]P |
| | 121) | | D[K] (SEQ ID NO: 123) |
| DHR25_design | DERDKVRELIDRVEKELKRE | DEAIKVAKEIVRVILELVRE | EEAIKKAKEIVRRILELTREG |
| | GTSEELIEEIRKVLKKAKEA | GTSSELIEEILKVLSLAAEAA | TSEEEIREELKELRKKAQKA |
| | ADSDD (SEQ ID NO: 130) | KSTD (SEQ ID NO: 131) | KSPE (SEQ ID NO: 132) |
| DHR25_variants | D[T]E[DK]R[A]D[KE]K[E]V | D[E]E[KD]AIK[E]V[A]AK[QY | E[DR]E[DS]AIK[RE]K[IEQ]A |
| | [A]R[EKS]E[K]LID[EKQ]R[EK | E]E[L]IV[A]R[EKD]V[A]IL[A | K[RYE]E[KR]IV[A]R[EKD]R |
| | Q]V[A]E[KR]K[E]E[QL]LK[Q | KR]E[LR]LV[AT]R[EK]E[SQ | [T]IL[AKR]E[R]LT[VAS]R[QK |
| | E]R[K]E[RSQ]GT[EQK]S[D]E | R]GT[EKQ]S[D]S[P]E[KRS]LI | E]E[RKD]GT[EQR]S[DNT]E[S |
| | [SPD]E[DNR]LIE[KTN]E[QA | E[QKR]E[QKD]ILK[ER]VLS | P]E[DN]E[KDQ]IR[SEK]E[K] |
| | D]IR[Q]K[ER]VLK[DRT]K[LE | [AEK]L[EK]AAE[KLR]AAK[N | E[TQR]LK[E]E[KQ]LR[AEK] |
| | N]AK[QDE]E[KQS]AAD[NKR] | RA]S[A]T[SP]D[N] (SEQ ID | K[E]K[REQ]AQ[KER]K[E]AK |
| | S[S]D[N] (SEQ ID NO: 127) | NO: 128) | [ANR]S[K]P[S]E[D] (SEQ ID |
| | | | NO: 129) |
| DHR26_design | DECERLRQEVEKAEKELEK | DECLRLASEVVKAVQELVK | EECLREASEVVKEVQELVK |
| | LAKQSTDEEVRQIAREVAK | LAEQATDEEVIRVALEVARE | EAEKSTDEEEIRELLQRAEE |
| | QLRRLAEEACRSNS (SEQ ID | LIRLAQEACRSND (SEQ ID | RIREAQERCREGD (SEQ ID |
| | NO: 136) | NO: 137) | NO: 138) |
| DHR26_variants | D[NT]E[DK]CE[KD]R[KE]LR | D[KPE]E[DNK]CL[I]R[KE]LA | E[DKP]E[NSD]CL[I]R[KEN]E |
| | [NQ]Q[EKT]E[ADK]VE[KDQ] | S[EKR]E[QR]VV[A]K[EQR]A | [T]AS[EAY]E[KQ]VV[A]K[E |
| | K[RS]AE[QKI]K[EDR]E[ALK] | V[A]Q[KER]E[LKA]LV[A]K | QR]E[RKS]V[A]Q[KER]E[K]L |
| | LE[NKQ]K[ERD]L[VA]A[K]K | [EDQ]L[VA]AE[KRA]Q[KNE] | V[A]K[EQ]E[KQR]AE[KLR]K |
| | [RDQ]Q[KNE]S[A]T[N]D[N]E | A[S]TDE[P]E[KNQ]V[AIL]IR | [R]S[AD]TD[N]E[P]E[NDQ]E |
| | [P]E[NDR]V[AIL]R[I]Q[KNR] | [KE]V[LIK]AL[A]E[RDK]VAR | [KRS]IR[K]E[KR]L[AD]L[A]Q |
| | I[LEK]AR[KQ]E[KTD]VAK[E | [AEL]E[LAR]LIR[EKN]LAQ | [KER]R[EKQ]AE[ALQ]E[KRD] |
| | D]Q[EAL]LR[EKQ]R[EKQ]LA | [YAL]E[LIK]ACR[EK]S[QNE] | R[EQT]IR[KEN]E[K]AQ[EA |
| | E[RDK]E[LDH]ACR[KN]S[N | N[GR]D[N] (SEQ ID NO: 134) | Y]E[K]R[KNQ]CR[KEQ]E[KN |
| | QE]N[G]S[D] (SEQ ID NO: | | R]GD[Q] (SEQ ID NO: 135) |
| | 133) | | |
| DHR27_design | TRQKEQLDEVLEEIQRLAEE | NEVIEKLLEVVKEIIRLAEEA | KERIEQLLREVKEEIRRAEEE |
| | ARKLMTDEEEAKKIQEEAE | MKKMTDEEEAAKIAKEALE | SRKETDDEEAAKRAREALR |
| | RAKEMLRRAVEKVTD (SEQ | AIKMLARAVEEVTD (SEQ | RIRERAREVEEDKS (SEQ ID |
| | ID NO: 142) | ID NO: 143) | NO: 144) |
| DHR27_variants | T[SD]R[EDK]Q[ATV]K[ED]E | N[VAD]E[DQN]V[AL]I[LV]E | K[NDE]E[DN]R[KQD]I[LV]E |
| | [KR]Q[REK]L[IA]D[KR]E[QT] | [KQR]K[ERQ]L[IA]L[A]]E[KH | [KR]Q[KRE]L[IAT]L[AI]R[ED |
| | V[A]L[IVE]E[K]E[R]IQ[KR]R | R]V[A]V[IA]K[ERQ]E[RL]IIR | K]E[KQR]V[IA]K[ELN]E[KR] |
| | [KE]L[A]AE[DK]EAR[A]K[RQ] | [E]L[A]AE[QK]E[RK]AM[A]K | E[I]IR[KE]R[EKN]AE[KQR]E |
| | L[RK]M[AE]T[SD]D[SNT]E | [ER]K[LR]M[A]T[ES]D[NT]E | [QRK]E[RKD]S[A]R[KED]K[R |
| | [DPS]E[NDK]E[KQR]AK[NQ] | [KDP]E[QK]E[DQR]AAK[ER]I | E]E[A]T[DS]D[NST]D[KPR]E |
| | K[ER]IQ[KI]E[KDN]E[QDK]A | A[I]K[ARE]E[KQ]ALE[KQR] | [QN]E[KDR]AAK[ERN]R[IE]A |
| | E[K]R[KEQ]AK[IQ]E[KQR]M | AIK[A]M[ADL]L[IQ]AR[AE] | [I]R[KAL]E[KQR]ALR[QEK] |
| | [ADL]L[IT]R[KED]R[DQE]A | AV[A]E[IK]E[QD]V[I]T[Q]D | R[KDQ]IR[AK]E[KQN]R[ETH] |
| | V[SAH]E[KR]K[QE]V[I]T[DE] | [N] (SEQ ID NO: 140) | AR[KND]E[KRD]V[AE]E[RQ |
| | D[N] (SEQ ID NO: 139) | | K]E[KR]D[EKR]K[TDQ]S[DN |
| | | | G] (SEQ ID NO: 141) |
| DHR28_design | DEEVQRIREEVRRAIEEVRE | DLAIEAIRALVRLAIEIVRLA | ELAKEAIRALRRLAEEIRRL |
| | SLERNDSEEAEELAREALER | LEQNDSELAREVAEEALRA | AEEQNDDELAREVEELARE |
| | VAEEVKESIKERPDR (SEQ | VAEVVKEAIRQRGDR (SEQ | AIEEVRKELERQRPGR (SEQ |
| | ID NO: 148) | ID NO: 149) | ID NO: 150) |
| DHR28_variants | D[TN]E[D]E[DNQ]V[IRK]Q[E | D[EQ]L[IVE]AI[EKQ]E[KQ]A | E[DS]L[IVE]AK[ED]E[KRD]A |
| | KR]R[KN]I[AL]R[KE]E[NKQ] | I[AL]R[KE]A[V]LV[A]R[EK]L | I[LEA]R[KQ]A[LV]LR[EKI]R |
| | E[TQ]V[A]R[KE]R[KQE]AI[A | [AT]AI[VAE]E[RQ]I[AL]V[IA] | [E]L[AT]AE[RK]E[RT]I[AL]R |
| | VK]E[RKQ]E[DKQ]V[IA]R[K | R[KEQ]L[E]ALE[KDQ]QN[G] | [IVA]R[KN]L[E]AE[KQ]E[KQ |
| | EQ]E[KDR]S[A]LE[DKR]R[E | D[N]S[P]E[DKQ]L[V]AR[EL | D]Q[H]N[G]D[N]D[PSQ]E[DK |
| | KN]N[G]D[N]S[PT]E[D]E[K]A | A]E[RKN]V[IA]AE[KQR]E[K | R]L[V]AR[EKQ]E[RKN]V[IA] |
| | E[ALK]E[K]L[IR]AR[EKQ]E | T]ALR[KE]AV[I]AE[QS]V[A] | E[KR]E[RK]L[EQN]AR[EDK] |
| | [KNQ]ALE[KDR]R[KTQ]V[I]A | V[A]K[Q]EA[I]IR[K]QR[A]G | E[RKQ]AI[V]E[KNR]E[R]V[A] |
| | E[RQK]E[IQA]V[A]K[R]E[RK] | [P]D[N]R[T] (SEQ ID NO: 146) | R[QED]K[ERN]E[QTV]L[RE |
| | S[ATI]IK[RQ]E[KNQ]R[HAK] | | K]E[K]R[KEN]Q[E]R[A]PG[N] |
| | PD[NG]R[TS] (SEQ ID NO: | | R[T] (SEQ ID NO: 147) |
| | 145) | | |
| DHR29_design | SEVEESAQEVEKRAQEVREE | SEVAESALQVVREALKVVL | SETARRALEKVRESLKEVLE |
| | AERRGTSQEVLDEIKRVVDE | SALERGTSEEVLKEILRVVS | QLERGTSEEELRESLREVSE |
| | ARQLAQRAKESDD (SEQ ID | EAIKLALEAIKSSD (SEQ ID | NIRKALEEIKSPD (SEQ ID |
| | NO: 154) | NO: 155) | NO: 156) |
| DHR29_variants | S[TD]E[DKR]V[ALT]E[KR]E | S[QEK]E[DKR]V[A]AE[KA]S | S[QER]E[DK]T[EDQ]AR[EKL] |
| | [KQT]S[ALQ]AQ[RED]E[KRQ] | [AEK]ALQ[EKR]V[A]V[IAL]R | R[KED]ALE[KR]K[ED]V[IA |
| | V[A]E[IKQ]K[DE]R[ELA]A | [AEK]E[ALK]A[L]L[W]K[QL | L]R[AKE]E[KR]S[AL]L[W]K |
| | [L]Q[KED]E[KRN]V[AL]R[EI | R]V[AL]V[ALI]L[IQR]S[QEA] | [ENQ]E[KDQ]V[ALI]L[IQA]E |
| | K]E[KQ]E[QRD]AE[KRQ]R[K | ALE[KQR]R[QET]GT[V]SE[D | [KQR]Q[ADR]L[Q]E[KRN]R[K |
| | DE]R[QTE]GT[V]SQ[SDP]E[D] | RW]E[DK]V[A]L[IV]K[RAD] | DQ]GT[KER]SE[DPR]E[DK]E |
| | V[AT]L[IQV]D[KRN]E[QDA] | E[LKD]IL[I]R[EKT]V[A]V[IA] | [QDK]L[IV]R[AKN]E[K]S[IQ |
| | IK[QEI]R[KEQ]V[A]V[IA]D[K | S[KQA]E[RLN]A[V]I[L]K[RE] | T]L[I]R[KE]E[KR]V[IA]S[EK |
| | ER]E[DKL]A[VL]R[KQE]Q[E | L[AV]A[ILV]L[EKQ]E[QIK]A | Q]E[KR]N[RTV]I[L]R[KEN]K |
| | R]L[AV]A[ILV]Q[KE]R[ELQ] | I[L]K[NRD]S[AL]S[T]D[NS] | [QRE]A[ILV]L[EIK]E[KR]E[D |
| | AK[RNE]E[K]S[AL]D[STQ]D | (SEQ ID NO: 152) | NK]I[L]K[RNQ]S[R]P[ST]D[S] |
| | [NS] (SEQ ID NO: 151) | | (SEQ ID NO: 153) |
| DHR30_design | STVKELLDRARELMRELAE | SEVIRLIAKAIMLMAELALR | KEEIRKVAEEIMRRAKTALD |
| | RASEQGSDEEEARKLLEDLE | AAEQGSDAEEAMKLLKDLL | EARQGSDAEEAMKRLKEQL |
| | QLVQEIRRELEETGTS (SEQ | RLVLEILRELRETGTD (SEQ | RRILERLREEREKGTD (SEQ |
| | ID NO: 160) | ID NO: 161) | ID NO: 162) |
| DHR30_variants | S[NT]T[DEK]V[AIT]K[ED]E | S[NKD]E[KNR]V[A]I[KAQ]R | K[DPQ]E[DQT]E[QT]I[KAQ] |
| | [KRN]L[A]L[E]D[KNE]R[KE] | [KIE]L[A]I[V]AK[E]AIM[A]L | R[KEI]K[REN]V[TDN]AE[KR] |
| | AR[KEL]E[K]L[R]M[AL]R[E | M[AL]AE[KQR]L[A]AL[AV]R | E[KRT]IM[A]R[DE]R[ALK]A |
| | K]E[KQ]L[A]AE[KRD]R[QEL] | [EKL]AAE[KDR]Q[ED]GS[A | K[REQ]T[EQD]AL[VKN]D[E |
| | AS[AKR]E[KR]Q[ED]GS[AQ | Q]D[NT]AE[AK]E[KR]AM[A | RK]E[RQK]AR[KED]Q[KDR] |
| | N]D[TN]E[PSK]E[DKN]E[RK] | L]K[Q]LLK[RI]D[EK]L[IV]LR | GS[EQD]D[NT]AE[KAR]E[K |
| | AR[KNQ]K[EQ]LLE[KD]D[E | [E]L[A]V[I]L[A]E[RK]IL[I]R | DQ]AM[AL]K[EQ]R[EKN]LK |
| | K]L[IV]E[QKR]Q[ERK]L[A]V | [EQD]E[AL]LR[KET]E[KR]T | [IRL]E[KD]Q[EKR]LR[EK]R |
| | [IE]Q[KED]E[RDK]IR[QKN]R | [AS]GTD[ST] (SEQ ID NO: | [KN]I[QT]L[A]E[RK]R[EK]L[I] |
| | [EK]E[ALQ]LE[DK]E[DKR]T | 158) | R[EKQ]E[KNR]E[RKL]R[K]E |
| | [SA]GT[A]S[TD] (SEQ ID | | [KDN]K[QEN]GTD[T] (SEQ ID |
| | NO: 157) | | NO: 159) |
| DHR31_design | DSYTERARKAVKRYVKEEG | SYLIQAAAAVVAYVIEEGGS | RELIRRAAERVAEVIERGGS |
| | GSEEEAEREAEKVREEIRKK | PEEAVKIAEEVVRRIKEKAD | PEEAVKEAEKEVKKQKEES |
| | ASD (SEQ ID NO: 166) | D (SEQ ID NO: 167) | D (SEQ ID NO: 168) |
| DHR31_variants | DS[D]Y[A]T[E]E[KR]R[QEK] | S[DQ]Y[A]LI[L]Q[ER]AAAA | R[D]E[DS]LI[L]R[EKQ]R[KQ |
| | AR[AN]K[ERD]A[L]V[A]K[AI | V[A]V[AI]AY[W]V[A]I[L]E[K | S]AAE[KQR]R[QEK]V[AI]AE |
| | E]R[KDE]Y[W]V[AT]K[ERQ] | N]E[KQ]GG[QY]S[DT]PE[D]E | [RDK]V[AEQ]I[L]E[KR]R[KQ |
| | E[K]E[KQ]GG[QY]S[T]E[P]E | [DR]AV[A]K[RE]I[ERQ]AE[R | N]GG[KNQ]S[T]PE[D]E[QKR] |
| | [D]E[QR]AE[KR]R[KE]E[DIN] | S]E[KQR]V[L]VR[EK]R[KE]I | AV[A]K[RQ]E[N]AE[K]K[RE] |
| | AE[KN]K[ER]V[L]R[VEK]E | [AL]K[EQ]E[KNT]K[NQ]AD[N | E[LQR]VK[R]K[ER]Q[ED]K[E |
| | [KQR]E[KR]I[AL]R[EK]K[DN | R]D (SEQ ID NO: 164) | Q]E[KNQ]E[KDN]S[R]D[TN] |
| | Q]K[QE]AS[NDE]D (SEQ ID | | (SEQ ID NO: 165) |
| | NO: 163) | | |
| DHR32_design | SIQEKAKQSVIRKVKEEGGS | STLVRAAAAVVLYVLEKGG | EELIREAAKEVLKVLEEGGS |
| | EEEARERAKEVEERLKKEA | STEEA VQRAREVIERLKKEA | VEEAVERARERIEELQKRSD |
| | DD (SEQ ID NO: 172) | SD (SEQ ID NO: 173) | D (SEQ ID NO: 174) |
| DHR32_variants | S[DT]I[TAQ]Q[E]E[DKQ]K[R | S[D]T[AQ]LV[IKA]R[KL]AA | E[D]E[DSK]L[EQ]I[VKA]R[KI |
| | DQ]AK[A]Q[NRE]S[A]VI[R]R | AAVVL[YAW]Y[WA]V[A]LE | N]E[KST]AAK[NQR]E[VQR] |
| | [KE]K[WY]V[AE]K[QER]E[K | [QK]K[EQ]GG[Y]S[ND]T[V]E | VL[YAW]K[EQ]V[AT]LE[DN |
| | QN]E[QKR]GG[KY]S[ND]E[D] | [D]E[T]AV[IL]Q[KRE]R[IKQ] | Q]E[RKD]GG[Y]S[ND]V[T]E |
| | E[D]E[KQ]AR[KQ]E[KRN]R | AR[EK]E[QR]V[AT]IE[KR]R | [DQ]E[Q]AV[L]E[KTD]R[E]A |
| | [EKL]AK[E]E[RKQ]V[AT]E[IR | [DKN]L[I]K[EQ]K[NT]E[KQD] | R[EK]E[KQR]R[QEA]IE[RK]E |
| | Q]E[RKQ]R[DEI]L[I]K[RQ]K | AS[NDK]D[S] (SEQ ID NO: | [KR]L[ERD]Q[EKS]K[TEN]R |
| | [RTD]E[KNS]AD[KNE]D[ST] | 170) | [KEN]S[AR]D[TN]D (SEQ ID |
| | (SEQ ID NO: 169) | | NO: 171) |
| DHR33_design | SETEEVKKLVEEKVKKEGG | STLLKVAALVASAVLKEGG | EELLKEAARQAEESLRQGKS |
| | SPEEAKETAKEVTEELKEES | SPEEAAETAKEVVKELRKSA | PEEAAEEAKKEVKKLKEKS |
| | QD (SEQ ID NO: 178) | SD (SEQ ID NO: 179) | QD (SEQ ID NO: 180) |
| DHR33_variants | S[DTN]E[AL]T[ELS]E[K]E[K | S[DE]T[LAE]LL[AR]K[EQR] | E[D]E[KDN]LL[AR]K[EQ]E[K |
| | D]VK[AN]K[ER]L[R]V[A]E[A | VAALV[A]AS[AK]A[WEL]V | RD]AAR[KEQ]Q[VRE]AE[A] |
| | K]E[KRQ]K[WQA]V[AT]K[Q | [A]LK[DE]E[QDK]GG[Q]S[NT | E[KRQ]S[VAT]LR[KEQ]Q[RK |
| | R]K[NDQ]E[QRD]GG[KQ]S[N | D]PE[D]E[Q]AA[V]E[KR]T[K | D]GK[GQ]S[NTD]PE[D]E[QR] |
| | D]P[D]E[D]E[RQ]AK[QE]E[K | QE]AK[ERA]E[R]V[A]VK[RD | AA[V]E[KR]E[NR]AK[AER]K |
| | QR]T[EKL]AK[DR]E[RK]V[A] | E]E[RKD]LR[TK]K[DER]S[Q | [RE]E[QHR]VK[ER]K[EQR]L |
| | T[VER]E[KD]E[RKD]LK[RT] | AT]AS[QH]D (SEQ ID NO: | [ENQ]K[TNQ]E[KNR]K[RE]S |
| | E[RKT]E[AQN]S[A]Q[DHR]D | 176) | Q[T]D[K] (SEQ ID NO: 177) |
| | [ST] (SEQ ID NO: 175) | | |
| DHR35_design | SEEDEVAKQASRYAKEQGG | SEALQVALEAARYASEEGE | EEDLKEALDRAREASERGQ |
| | DPEKSREEAEKALEEVKKQ | DPAEALKEAARALEEVRRS | NPAESLKEAAEELKKKKEK |
| | ATS (SEQ ID NO: 184) | ATS (SEQ ID NO: 185) | SSD (SEQ ID NO: 186) |
| DHR35_variants | S[NT]E[DT]E[QKR]D[EKQ]E | S[D]E[D]A[D]L[EIK]Q[KR]V | E[D]E[D]D[A]L[EKI]K[QE]E |
| | [KQ]V[A]AK[REQ]Q[ELW]AS | [A]ALE[LW]AAR[KE]Y[W]AS | [KRQ]ALD[KER]R[E]AR[KDE] |
| | [A]R[EKD]Y[W]AK[SQR]E[K | [YHR]E[KNQ]E[Q]GE[Q]D[N] | E[RK]AS[YAQ]E[KNQ]R[ED |
| | NR]QGG[QH]D[N]PE[N]K[ED | PAE[DK]ALK[EQR]E[R]AAR | Q]GQ[E]N[D]PAE[DQ]S[A]L |
| | Q]S[A]R[LK]E[K]E[KDR]AE | [KE]ALE[K]E[QK]V[A]R[KN] | K[EHQ]E[KRQ]AAE[KR]E[K |
| | [KRN]K[ER]ALE[K]E[LQ]V[A] | R[K]S[A]AT[E]S[T] (SEQ ID | R]LK[E]K[EQR]K[ERQ]K[SN] |
| | K[REN]K[R]Q[A]AT[QS]S[T] | NO: 182) | E[KR]K[E]SS[TQ]D[RT] (SEQ |
| | (SEQ ID NO: 181) | | ID NO: 183) |
| DHR36_design | SDLEKALKRFVKEEKKKGR | SDLLTALAKFVLEEVRKGR | SEQLEKLATKVLEEVKKGR |
| | NPEEAKKEAKKLKKKLKKS | NPEEAVKEAIKLAEKLKRSA | NPKRAVEEAIKQAKEDRKR |
| | AGS (SEQ ID NO: 190) | GS (SEQ ID NO: 191) | SNS (SEQ ID NO: 192) |
| DHR36_variants | S[T]D[EN]LE[DK]KALK[NEQ] | S[D]D[AKN]LLT[KEQ]ALAK | S[D]E[DQS]Q[E]LE[RKT]K[E] |
| | R[QEN]F[Y]V[I]K[RED]E[D | [TDR]F[Y]VLE[QD]E[Q]VR[K | LAT[KRE]K[ESH]VLE[K]E[R |
| | QK]E[Q]K[ET]K[DRE]KGR[Q | EQ]KGR[KQ]N[TD]PEE[K]A | AL]VK[QE]K[R]GR[EQT]N[T |
| | K]N[DTS]P[ER]E[DKQ]E[KD | VK[R]E[S]AIK[E]LAE[Q]K[N | D]PK[E]R[EDK]AVE[RK]E[K |
| | Q]AK[R]K[RED]E[SD]AK[ER] | R]LK[RQ]R[KN]S[A]AGS | DR]AIK[ER]Q[EKN]AK[EQ]E |
| | K[E]LK[ER]K[ER]K[RD]LK | (SEQ ID NO: 188) | [KR]D[RKE]R[KN]K[REN]R |
| | [RE]K[RNT]S[A]AGS (SEQ ID | | [KTN]S[K]N[QT]S[D] (SEQ ID |
| | NO: 187) | | NO: 189) |
| DHR37_design | SSTERAAQSVKKYLQQQGK | SSVIRAAAAVVFYLLEQGY | DDVIKEAAKVVYKRLEEGQ |
| | DPDQAQKKAQEVKENIEKE | DPDQALKKAQEVARNIENE | DPDKALEEARKRAQKTEKK |
| | ANS (SEQ ID NO: 196) | ANS (SEQ ID NO: 197) | TTS (SEQ ID NO: 198) |
| DHR37_variants | S[TD]S[AQ]T[SA]E[KQD]R[K | S[DE]S[AE]V[A]IR[KES]AAA | D[E]D[ESQ]V[A]IK[RE]E[RT |
| | E]AAQ[RDK]S[AE]VK[IRY]K | A[E]VVF[EKI]YLLE[RNQ]QG | A]AAK[ERS]VVY[EIK]K[E]R |
| | [ER]YLQ[K]Q[REK]QGK[YG | Y[Q]D[S]P[A]D[E]Q[KER]AL | [LE]LE[KQR]E[RK]GQ[YKR] |
| | R]D[SN]P[S]D[E]Q[E]AQ[ED | K[ER]K[QVE]AQ[RI]E[KR]V | D[S]P[A]D[E]K[QDE]ALE[KQ] |
| | K]K[R]K[VQ]AQ[RED]E[Q]V | AR[KNQ]N[ADQ]IEN[KDE]E | E[KQR]AR[IQ]K[ER]R[EHQ] |
| | K[AQ]E[KT]N[QAD]IE[T]K[E] | [QT]ANS[T] (SEQ ID NO: | AQ[KER]K[ENR]T[EKI]EK[R |
| | E[QT]AN[T]S[T] (SEQ ID | 194) | EN]K[ETQ]T[EKR]TS[DT] |
| | NO: 193) | | (SEQ ID NO: 195) |
| DHR39_design | SDLQEVADRIVEQLKREGRS | SELIEVAVRIVKELEEQGRSP | SDRIKKAVELVRELEERGRS |
| | PEEARKEARRLIEEIKQSAG | SEAAKEAVELIERIRRAAGG | PSEAARRAVEEIQRSVEEDG |
| | GD (SEQ ID NO: 202) | D (SEQ ID NO: 203) | GN (SEQ ID NO: 204) |
| DHR39_variants | S[ND]D[EKN]L[TED]Q[KD]E | S[EDQ]E[DQN]LI[R]E[RDQ]V | S[DP]D[KE]R[L]IK[EQR]K[R |
| | [KNR]V[I]AD[KE]R[KEN]IV[I | [I]AV[AI]R[QEW]IV[I]K[EQ] | E]AV[AI]E[K]L[ET]V[I]R[KE |
| | R]E[KR]Q[AD]L[A]K[EQR]R | E[QAD]L[A]E[QTI]E[KNQ]Q | D]E[KQ]L[AE]E[QAN]E[KRN] |
| | [KN]E[DKN]GR[QHK]S[DN]P | [DKE]GR[QY]S[DN]P[A]S[AR] | R[KED]GR[QKN]S[DN]P[A]S |
| | [ER]E[DN]E[S]AR[EK]K[RE]E | E[R]AAK[ER]E[TK]AV[A]E[R] | [AR]E[KDN]AAR[EK]R[EKQ] |
| | [TKQ]AR[DEK]R[EK]LI[V]E | LI[V]E[KQR]R[K]IR[V]R[DE | AV[A]E[RQ]E[RDK]I[V]Q[EK |
| | [KRN]E[KRQ]IK[RQ]Q[DKE]S | Q]AAGGD[N] (SEQ ID NO: | A]R[KNE]S[DER]VE[RKD]E |
| | [A]AGGD[NT] (SEQ ID NO: | 200) | [KNR]D[NQ]GGN (SEQ ID |
| | 199) | | NO: 201) |
| DHR40_design | SESDEVAKRISKEAKKEGRS | SEAIRVAVEIADEALREGLSP | EDEIQKAVETAQEQLEEGRS |
| | EEEVKELVERFREAIEKLKE | EEVVELVERFVQAIQKLQEN | PKEVVETVEEQVKEVEEKQ |
| | QGD (SEQ ID NO: 208) | GE (SEQ ID NO: 209) | QKGE (SEQ ID NO: 210) |
| DHR40_variants | S[TD]E[DKQ]S[A]D[EK]E[K] | S[EDK]E[DKR]AI[EKV]R[EQ | E[DKS]D[E]E[SAR]I[EKV]Q |
| | V[A]AK[QE]R[KN]IS[AEK]K | K]V[A]AVE[RKQ]IAD[E]E[Q | [EK]K[RQ]AVE[RKQ]T[IED]A |
| | [ER]E[QL]AKK[R]E[DKQ]GR | L]AL[Q]R[K]E[DK]GL[KRA]S | Q[EI]E[KNQ]Q[A]L[Q]E[RDK] |
| | [KAE]S[D]E[P]E[DK]E[QR]VK | [D]P[A]E[KQ]E[QRT]VV[A]E | E[DTK]GR[KAE]S[DN]P[A]K |
| | [NQ]E[K]LV[A]E[KR]R[D]F | [R]LVE[IKQ]R[E]F[Y]V[A]Q | [ER]E[QKS]VV[A]E[RK]T[DR |
| | [Y]R[KEQ]E[KQD]AI[L]E[KQ | [KRD]AI[L]Q[ENK]K[DQ]LQ | N]VE[Q]]E[RK]Q[HES]V[A]K |
| | D]K[ER]LK[QRE]E[KRD]Q[N | [RE]E[KQR]N[EQ]GE[ND] | [ET]E[KNR]V[EIN]E[DQK]E |
| | ED]GD[N] (SEQ ID NO: 205) | (SEQ ID NO: 206) | [KR]K[EL]Q[DEK]Q[KRD]K[E |
| | | | QR]GE[QKN] (SEQ ID NO: |
| | | | 207) |
| DHR41_design | SDIEKAKRIADRAIDVVRKA | SDVREAARVALEAVRVVVR | ENVRESARRALEKVLKTVQ |
| | AEKEGGSPEKIREALQQAKR | AAEEKGGSPEEVVEAVCRA | QAEEEGKSPEEVVEQVCRS |
| | CAEKLIRLVKEAQESNS | VRCAEKLIRLVKRAEESNS | VRKAEEQIRETQERERSTS |
| | (SEQ ID NO: 214) | (SEQ ID NO: 215) | (SEQ ID NO: 216) |
| DHR41_variants | S[DT]D[NLR]I[ARE]E[KDR]K | S[DEQ]D[NA]V[A]R[QK]E[K | E[DQT]N[RDE]V[A]RE[KR]S |
| | [ER]AK[ER]R[KE]I[V]AD[KE | RQ]AAR[EKQ]V[I]AL[I]E[RD | [AR]AR[KQE]R[KE]AL[I]E[K] |
| | Q]R[EK]AI[VE]D[EKR]V[AI] | Q]AVR[EK]V[AI]V[A]VR[EK] | K[HDE]VL[ER]K[ER]T[V]VQ |
| | V[A]R[QED]K[ER]AAE[KDR] | AAE[Q]E[KR]K[RET]GGS[D | [REK]Q[KEN]AE[QKS]E[KR] |
| | K[NR]E[KQR]GGS[D]P[SE]E | N]P[A]E[DKR]E[DQR]V[I]V | E[DKR]GK[G]S[D]P[A]E[DR |
| | [DNQ]K[ER]IR[KDQ]E[QKR]A | [A]E[R]AV[I]C[EAI]R[E]AV[A] | K]E[KD]V[I]V[A]E[R]Q[RND] |
| | L[EIR]Q[KDE]Q[ERD]AK[RE] | R[EK]C[AV]AE[RK]K[ERL]L | V[I]C[EKQ]R[EK]S[A]V[A]R |
| | R[EK]C[AV]AE[KR]K[RL]L | [AI]I[VL]R[EKD]L[IAV]V[A] | [EK]K[QR]AE[AKQ]E[KRQ]Q |
| | [AI]I[KLR]R[KE]L[IAV]V[A] | K[EAQ]R[EDK]AE[Q]E[RDK] | [EDR]I[VL]R[KEQ]E[KTD]T[Q |
| | K[EQ]E[KRQ]AQ[EDK]E[RDK] | S[D]N[PSQ]S[N] (SEQ ID NO: | VA]Q[EAK]E[KNT]R[KE]E[Q] |
| | S[LAK]N[PS]S[N] (SEQ ID | 212) | R[KDE]S[RK]T[NPS]S[DN] |
| | NO: 211) | | (SEQ ID NO: 213) |
| DHR42_design | SDAEEVKKQAEEIANRAYK | SDALEVARQALEIARRAFET | QKALEIARKALQKAKENFE |
| | TAQKQGESDSRAKKAEKLV | AKKQGHSATEAAKAFVDV | EAQKRGESATQAAKRFVDT |
| | RKAAEKLARLIERAQKEGD | VEAAISLAELIISAKRQGD | VEKEIKKAQEQIKRERKGD |
| | (SEQ ID NO: 220) | (SEQ ID NO: 221) | (SEQ ID NO: 222) |
| DHR42_variants | S[DT]D[TIQ]A[S]E[KQ]E[KR | S[DEQ]D[TEI]AL[AEK]E[KQ | Q[DER]K[EDT]AL[EK]E[KQR] |
| | Q]V[I]K[ERQ]K[ED]Q[EDK]A | R]V[I]AR[E]Q[EI]AL[A]E[K]I | I[V]AR[ES]K[EQ]AL[A]Q[EK |
| | E[KR]E[K]I[LT]AN[EQK]R[Q | [LT]AR[KEI]R[KDE]AFE[KR] | R]K[RD]AK[ELR]E[RK]N[AE] |
| | KE]AY[EKR]K[EDR]T[QER] | T[EQ]AK[RNT]K[R]Q[DER]G | FE[KQR]E[QKN]AQ[REN]K |
| | AQ[KRE]K[EQR]Q[DE]GE[Q | H[QLE]SAT[QR]E[QR]AAK[E] | [NR]R[DKQ]GE[KLR]S[D]AT |
| | HK]S[D]D[EPS]S[DK]R[EQ]A | AF[Y]V[AEQ]D[TAL]VVE[R | E[QR]Q[ER]AAK[QE]R[EA]F |
| | K[QDE]K[Q]AE[YFR]K[EDR] | KD]AAI[K]S[KEQ]LAE[QRT] | [Y]V[AEK]D[ER]T[VR]VE[KD |
| | L[TDA]VR[EKL]K[RE]AAE[R | LI[A]I[EL]S[KEQ]AK[QRE]R | R]K[E]E[A]I[REK]K[E]K[E]A |
| | KD]K[EQR]LAR[EKQ]LI[A]E | [KQ]Q[ED]GD[NS] (SEQ ID | Q[ERK]E[KR]Q[ASE]I[RLN]K |
| | [KR]R[KE]AQ[ERK]K[DER]E | NO: 218) | [ERQ]R[LE]E[QDK]R[KEQ]K |
| | [QN]GD[NS] (SEQ ID NO: | | [ER]GD[QKT] (SEQ ID NO: |
| | 217) | | 219) |
| DHR43_design | SKEEELIEKARRVAKEAIEE | SELAELISEAIQVAVEAVEE | SELAKKINDTIREAVREVQQ |
| | AKRQGKDPSEAKKAAEKLI | AVRQGKDPFKAAEAAAELI | AVEDGKDPFEAAREAAEKI |
| | KAVEEAVKEAKRLKEEGN | RAVVEAVKEAERLKREGN | RESVERVREEEEKKRRGN |
| | (SEQ ID NO: 226) | (SEQ ID NO: 227) | (SEQ ID NO: 228) |
| DHR43_variants | S[TD]K[ETD]E[L]E[KD]E[KN | S[EQT]E[DK]LAE[RKD]LIS[E | S[KET]E[DK]LAK[RDE]K[ER] |
| | Q]LIE[KR]K[ER]AR[E]R[EKQ] | KR]E[KR]AIQ[REK]V[AT]AV | IN[KRE]D[EKQ]T[AS]IR[EK |
| | V[AT]AK[ER]E[KRN]A[L]I | [I]E[RDQ]A[L]VE[DKQ]E[QT | Q]E[QK]AV[IL]R[KEQ]E[DN |
| | [V]E[KDR]E[KQT]AK[QRE]R | R]AV[QRA]R[KE]Q[DE]GK[Q | K]V[I]Q[E]Q[END]AV[QAN]E |
| | [KED]Q[DK]GK[QL]D[NS]P[E | L]D[N]P[A]F[WA]K[RED]AA | [KR]D[QKE]GK[Q]D[NT]P[A] |
| | S]S[DNT]E[KRL]AK[REQ]K | E[KR]AAAE[RK]LIR[KE]AV | F[WAT]E[DK]AAR[EKH]E[R |
| | [ER]AAE[KDR]K[ER]LIK[ENR] | V[A]E[KRD]AV[A]K[ERQ]E | KD]AAE[KQR]K[ERH]IR[EK |
| | AVE[R]E[KQR]AV[A]K[E]E | [VR]AE[RH]R[KQ]LK[ES]R[E | Q]E[KNQ]S[VET]V[A]E[KRD] |
| | [TVK]AK[ER]R[KE]LK[ER]E | K]E[NDK]GN (SEQ ID NO: | R[QED]V[A]R[QKE]E[KR]E |
| | [RKD]E[NQR]GN (SEQ ID NO: | 224) | [DKQ]E[AS]E[KR]K[RA]K[DR] |
| | 223) | | R[KEN]R[NEK]GN[KEQ] |
| | | | (SEQ ID NO: 225) |
| DHR44_design | SNEQEKKDLKKAEEAAKSP | NKAKEIILRAAEEAAKSPDP | EKAKEIIKRAAEEAQKSPDP |
| | DPELIREAIERAEESGS (SEQ | ELIRLAIEAAERSGS (SEQ ID | ELQKLAKEARERLG (SEQ ID |
| | ID NO: 232) | NO: 233) | NO: 234) |
| DHR44_variants | S[T]N[DT]E[DQ]Q[DE]E[KDN] | N[ED]K[REQ]AK[E]E[K]IILR | E[D]K[DEQ]AK[R]E[KR]IIK |
| | K[EQR]K[ER]D[RIK]LK[ER | [DEI]AAE[KR]E[V]AAK[DER] | [REL]R[ILT]AAE[DKR]E[VQ] |
| | D]K[RDE]AE[KQR]E[KQ]AA | S[A]P[ST]D[N]PE[DNQ]LI[L] | AQ[KE]K[RN]S[AE]P[SQ]D[N] |
| | K[ENR]SP[ST]D[N]PE[DNS]L | R[KDE]L[TKQ]AI[V]E[KR]A | P[E]E[DN]LQ[L]K[ER]L[KET] |
| | [KND]I[L]R[KDE]E[RKT]AI[L | [W]AE[KQR]R[E]S[Q]GS[T] | AK[EQR]E[KR]A[W]R[AEK] |
| | V]E[KDR]R[ELQ]AE[QKD]E | (SEQ ID NO: 230) | E[KRN]R[EKQ]L[QSE]G |
| | [KRQ]S[QET]GS[T] (SEQ ID | | (SEQ ID NO: 231) |
| | NO: 229) | | |
| DHR45_design | SSEEEELEKDAREASESGAD | SEVIELAKRALEAAKSGADP | EEVIELAKRALEEAKKGKDP |
| | PEWLREIVDLARESGD (SEQ | EWLLRIVRQAEESGS (SEQ | KELLEEVRKREESG (SEQ ID |
| | ID NO: 238) | ID NO: 239) | NO: 240) |
| DHR45_variants | S[DN]S[DT]E[D]E[TSD]E[KD] | S[DP]E[Q]V[A]I[K]E[K]LAK | E[PDK]E[QND]V[A]I[K]E[KR] |
| | E[KR]LE[QK]K[R]D[LKA]AR | [ES]R[LAK]ALE[QD]AAK[E]S | L[EA]AKR[EKQ]ALE[DRK]E |
| | [KD]E[S]AS[A]E[NK]S[T]GA | [T]GAD[NT]P[A]E[RKQ]W[A | [RD]AK[R]K[E]GK[QH]D[NT] |
| | D[TN]P[S]E[NT]W[ALY]LR[K | LY]LL[W]R[KQ]IVR[QDE]Q | P[A]K[REH]E[QDR]LL[W]E |
| | E]E[KR]IVD[REN]L[QTD]AR | [TE]AE[RST]E[KDN]S[E]GS[D | [KR]E[K]VR[QKA]K[ET]R[KN |
| | [KTS]E[KNR]S[Q]GD[NT] | N] (SEQ ID NO: 236) | S]E[T]E[KDR]S[RKE]G (SEQ |
| | (SEQ ID NO: 235) | | ID NO: 237) |
| DHR46_design | STKEEKERIERIEKEVRSPDP | TEAEELLRRAIEAAVRAPDP | EEAKELLRRAIESAKKAPDP |
| | ENIREAVRKAEELLRENPS | EAIREAVRAAEELLRENPS | EAQREAKRAEEELRKEDP |
| | (SEQ ID NO: 244) | (SEQ ID NO: 245) | (SEQ ID NO: 246) |
| DHR46_variants | S[D]T[D]K[DEQ]E[DKT]E[KL | T[DE]E[D]AE[KQR]E[KR]L[A | E[DQ]E[DK]AK[QER]E[KR]L |
| | Q]K[RED]E[KDR]R[TDK]I[E | I]LR[EAS]R[KE]AIE[RKQ]A | [AI]LR[EK]R[ETK]AIE[RKQ] |
| | AK]E[KR]R[EKD]IE[RDK]K | [RQ]AV[A]R[EKD]APD[N]P[A | S[AER]AK[QE]K[ERN]APD[N] |
| | [R]E[A]V[A]R[EKD]S[A]P[S] | D]E[SDK]AIR[KE]E[ALR]AV | P[SEK]E[DKN]AQ[R]R[KDE] |
| | D[N]P[ADS]E[DKN]N[EAD]IR | R[ED]AAE[RS]E[QH]LL[Y]R | E[AKL]AK[E]R[EQK]AE[QK |
| | [EK]E[KQR]AVR[EK]K[EAD] | [EK]E[NRD]N[D]P[D]S (SEQ | R]E[KR]E[QDR]LR[DKE]K[E |
| | AE[ARK]E[KR]LL[YA]R[KE | ID NO: 242) | R]E[NQD]D[N]P[D] (SEQ ID |
| | Q]E[KRN]N[D]P[D]S (SEQ | | NO: 243) |
| | ID NO: 241) | | |
| DHR48_design | NSREEEEAKRIVKEAKKSGF | SEALKEALKIVEEAAKSGYD | PEELKEALKRVLEAAKRGE |
| | DPEEVEKALREVIRVAEETG | PAEVAKALAEVIRVAEETG | DPAQVAKELAEEIRRNQEEG |
| | N (SEQ ID NO: 250) | N (SEQ ID NO: 251) | (SEQ ID NO: 252) |
| DHR48_variants | N[D]S[D]R[EDH]E[AS]E[KR] | S[PR]E[D]AL[A]K[ER]E[DKQ] | P[RQ]E[D]E[SAD]L[A]K[ENR] |
| | E[K]E[KDL]AK[ER]R[EK]I[V] | ALK[RED]I[V]V[A]E[KR]E | E[KR]ALK[ER]R[E]V[A]L[E |
| | V[A]K[E]E[QRK]AK[Q]K[E]S | [Q]AAK[ER]SGYD[N]P[A]AE | RS]E[KR]AAK[ER]R[KEQ]GE |
| | GF[Y]D[N]P[S]E[NTK]E[KQT] | [QD]VAK[RD]ALAE[KR]V[L]I | [KRT]D[N]P[A]AQ[DKE]VAK |
| | VE[KQ]K[RE]ALR[DEK]E[R | R[KE]VAE[Q]E[DR]T[HKS]G | [ED]E[K]LAE[KR]E[Q]IR[EK |
| | KQ]V[L]I[QR]R[EK]VAE[QR] | N[D] (SEQ ID NO: 248) | Q]R[EDK]N[ARD]Q[ET]E[RD |
| | E[RQ]T[KH]GN[D] (SEQ ID | | K]E[KR]G (SEQ ID NO: 249) |
| | NO: 247) | | |
| DHR49_design | DSEEEQERIRRILKEARKSGT | SEVLEEAIRVILRIAKESGSE | PRVLEEAIRVIRQIAEESGSE |
| | EESLRQAIEDVAQLAKKSQD | EALRQAIRAVAEIAKEAQD | EARRQAERAEEEIRRRAQ |
| | (SEQ ID NO: 256) | (SEQ ID NO: 257) | (SEQ ID NO: 258) |
| DHR49_variants | D[TS]S[T]E[D]E[DQ]E[WS]Q | S[P]E[DS]VL[W]E[KAR]E[RH | PR[NED]VL[W]E[KR]E[TAH] |
| | [KAE]E[KNR]R[KDN]I[A]R[K | L]AI[A]R[EDK]V[ERL]IL[AE | AI[KAQ]R[KE]V[ERQ]IR[QE |
| | EQ]R[KEN]I[T]L[AVW]K[EN | V]R[EK]I[AL]AK[DEQ]E[QD | K]Q[ERK]I[AL]AE[KDR]E[Q |
| | R]E[KND]AR[QTD]K[NR]S[D | R]S[A]GS[DN]E[DNP]E[DR]A | ND]S[A]GS[DN]E[DPS]E[DK] |
| | Q]GT[SDN]E[DKN]E[DS]S[A | [V]L[I]R[KAI]Q[ERK]AIR[ED | A[V]R[KEI]R[KE]Q[KEL]AE |
| | DQ]L[I]R[KIQ]Q[KER]AIE[K | Q]AV[I]AE[RDK]IAK[ERS]E | [KQR]R[EK]AE[IKQ]E[RDK]E |
| | DN]D[KRE]V[I]AQ[RKE]L[IE | [QDK]AQ[TND]D[NST] (SEQ | [RQT]IR[KDE]R[KD]R[QDK] |
| | V]AK[ERS]K[EDQ]S[A]Q[TN | ID NO: 254) | AQ[TND] (SEQ ID NO: 255) |
| | R]D[TS] (SEQ ID NO: 253) | | |
| DHR50_design | DPEEVRREVERATEEYRKNP | PEAVQVAVEAATQIYENTP | PEAVRVAEEAADQIRKNTP |
| | GSDEAREQLKEAVERAEEA | GSEEAKKALEIAVRAAENA | GSELAKRADEIKKRARELLE |
| | ARSPD (SEQ ID NO: 262) | ARLPD (SEQ ID NO: 263) | RLP (SEQ ID NO: 264) |
| DHR50_variants | D[SNT]P[ST]E[DS]E[DR]V[A | P[WAE]E[KD]AV[AL]Q[EDK] | P[ST]E[KDQ]AV[AL]R[KED] |
| | EL]R[KEL]R[KDE]E[TKI]V[A] | V[AT]AV[A]E[RKN]A[I]AT[K | V[TAK]AE[QKR]E[RKT]A[I] |
| | E[RKD]R[KED]AT[EKQ]E[K | QE]Q[IKR]I[V]Y[W]E[QDK]N | AD[KE]Q[IRT]I[V]R[WYI]K |
| | R]E[IRT]Y[W]R[KQD]K[E]N | [DT]T[E]PGSE[DQ]E[LAN]A | [QE]N[T]T[E]PGSE[D]L[AEN] |
| | [HRD]PGSD[ER]E[DK]AR[KE | K[ER]K[ERT]ALE[KR]I[LA]A | AK[E]R[EKT]AD[QKR]E[KN |
| | H]E[KR]Q[AS]LK[RE]E[KRQ] | VR[DE]AAE[R]N[AER]A[L]A | R]I[LA]K[A]K[E]R[EKQ]AR |
| | AVE[K]R[AD]AE[KQR]E[K]A | R[EN]L[SN]P[S]D[SNT] (SEQ | [EQK]E[KQR]L[NVA]L[KAR] |
| | [L]AR[KDE]S[LKN]P[S]D[SN | ID NO: 260) | E[KDN]R[KE]L[SAN]P[S] |
| | T] (SEQ ID NO: 259) | | (SEQ ID NO: 261) |
| DHR51_design | QSEDRKEKIRELERKARENT | ADTAKEAIQRLEDLARDYS | KETAEEAIKRLRELAEDYKG |
| | GSDEARQAVKEIARIAKEAL | GSDVASLAVKAIAKIAETAL | SEVAKLAEEAIERIEKVSRER |
| | EEGN (SEQ ID NO: 268) | RNGY (SEQ ID NO: 269) | G (SEQ ID NO: 270) |
| DHR51_variants | Q[HNK]S[DNT]E[D]D[EQT]R | A[SRK]D[E]T[V]AK[EIQ]E[H | K[RST]E[D]T[V]AE[KQ]E[HT |
| | [QAD]K[IQ]E[KR]K[DR]IR[K | T]AIQ[EKR]R[E]LE[AQK]D | Q]AI[K]K[RD]R[EQS]LR[QK |
| | QE]E[R]LE[AQK]R[KE]K[TI] | [K]L[IV]AR[EDS]D[KTE]Y[F] | E]E[KDS]L[IV]A[R]E[DKR]D |
| | AR[EKQ]E[KRT]N[YEH]T[S] | S[T]GS[T]D[ES]V[A]AS[RK]L | [KQE]Y[F]K[TED]GS[T]E[DQ] |
| | GS[T]D[E]E[DKR]AR[K]Q[KE | [E]AV[AI]K[ERQ]A[L]IAK[E | V[A]AK[E]L[EDQ]AE[KR]E |
| | N]AV[AI]K[QER]E[DKR]IAR | HR]IAE[KQ]T[VER]AL[A]R | [KQ]A[L]IE[KQR]R[EHK]IE[K |
| | [KED]IAK[ER]E[KQ]AL[A]E | [KEQ]N[Q]GY[ND] (SEQ ID | N]K[EDT]V[EIQ]S[A]R[K]E[K |
| | [KRQ]E[RK]GN[S] (SEQ ID | NO: 266) | R]R[QEN]G (SEQ ID NO: 267) |
| | NO: 265) | | |
| DHR53_design | SNDEKEKLKELLKRAEELA | NLAKKALEIILRAAEELAKL | ELAKKALEIIERAAEELKKSP |
| | KSPDPEDLKEAVRLAEEVV | PDPEALKEAVKAAEKVVRE | DPEAQKEAKKAEQKVREER |
| | RERPGS (SEQ ID NO: 274) | QPGS (SEQ ID NO: 275) | PG (SEQ ID NO: 276) |
| DHR53_variants | SN[DST]D[E]E[DTR]K[ED]E | N[EDS]L[NAK]AK[E]K[ERT] | E[SR]L[ANQ]AK[E]K[ER]AL |
| | [K]K[E]LK[ER]E[K]L[IRK]LK | ALE[KR]IILR[TKD]AAE[KR] | E[KDR]IIE[QK]R[ELT]AAE[K |
| | [DRE]R[K]AE[R]E[KQ]LAK[R | E[AN]LAK[EQR]LPD[NS]P[E] | RT]E[NA]LK[QER]K[R]S[L]P |
| | EN]S[L]PD[N]P[E]E[NDK]D | E[NRT]ALK[EQN]E[KRA]AV | D[NS]P[DE]E[QKN]AQ[KR]K |
| | [A]LK[ERQ]E[KR]AVR[DEK] | K[ER]AAE[K]K[DEQ]VV[I]R | [E]E[KRD]AK[E]K[RE]AE[K |
| | L[TAE]AE[KQ]E[KR]VV[I]R | [NDK]E[TQ]Q[RT]PGS (SEQ | NQ]Q[EKN]K[ERD]VR[K]E[D |
| | [EQK]E[Q]R[Q]P[S]GS (SEQ | ID NO: 272) | RK]E[TQ]R[QN]PG (SEQ ID |
| | ID NO: 271) | | NO: 273) |
| DHR54_design | TTEDERRELEKVARKAIEAA | TEAVKLALEVVARVAIEAA | EEAVRLALEVVKRVSDEAK |
| | REGNTDEVREQLQRALEIAR | RRGNTDAVREALEVALEIA | KQGNEDAVKEAEEVRKKIE |
| | ESGT (SEQ ID NO: 280) | RESGT (SEQ ID NO: 281) | EESG (SEQ ID NO: 282) |
| DHR54_variants | T[S]T[DNS]E[DQ]DE[WAD]R | T[DEK]E[KD]AV[WF]K[EDR] | E[DKP]E[RDK]AV[WF]R[KD |
| | [KEN]R[EK]E[KQN]L[I]E[KR | L[RK]AL[I]E[RD]V[A]V[AI]A | E]L[WER]AL[EIQ]E[KR]V[A] |
| | Q]K[E]V[AI]A[K]R[EKQ]K[R | [K]R[EKQ]V[A]AI[KAQ]E[A | V[AI]K[DEH]R[EKN]V[A]S[A] |
| | E]AI[KA]E[KQR]AAR[QEK]E | QR]AAR[Q]R[QK]GNT[ANR] | D[EKR]E[AKQ]AK[QED]K[E |
| | [KRD]GN[D]T[ANR]D[EK]E | D[E]AVR[EK]E[AIK]ALE[RK | NR]Q[RK]GNE[SRD]D[ES]A |
| | [RQ]VR[EKQ]E[RK]Q[A]LQ[E | IV[A]A[I]L[AIQ]E[QRK]I[A]A | V[AES]K[EDQ]E[LRA]AE[QR |
| | RD]R[KEN]A[I]L[ARI]E[KQR] | R[KND]E[DNK]S[A]GT[S] | K]E[KQ]V[AT]R[AEK]K[ER] |
| | I[AET]AR[NKE]E[KDQ]S[A | (SEQ ID NO: 278) | K[REH]I[A]E[KQR]E[KRN]E |
| | QT]GT[S] (SEQ ID NO: 277) | | [NDK]S[AK]G (SEQ ID NO: |
| | | | 279) |
| DHR55_design | SSVAEEIEKRAKKISKELKK | SDALEIAKRAVKIAEELAKQ | PKALKQAKEAVKEAEELAK |
| | EGKNPEWIEELQRAADKLV | GSNPKWIAELLKAAAKLVE | KGRNPKEIAEELKKRAKEVE |
| | EVARRATS (SEQ ID NO: 286) | VAARATS (SEQ ID NO: 287) | KLARST (SEQ ID NO: 288) |
| DHR55_variants | S[DN]S[NT]V[K]AE[DK]E[KT] | S[PE]D[VK]AL[R]E[K]IAK[E | P[SE]K[ED]AL[RW]K[RE]Q[I |
| | IE[RKA]K[E]R[TIK]AK[E]K[E] | QL]R[KLT]AV[A]K[EQR]IAE | TE]AK[EQ]E[KRD]AV[A]K[E |
| | IS[A]K[ER]E[H]LKK[R]E[Q] | [KLR]E[RDK]LAK[ERQ]Q[ER] | R]E[KQS]AE[K]E[KR]L[RKD] |
| | GK[AS]N[D]PE[TN]W[AK]IE | GS[A]N[D]PK[ES]W[AKQ]IA | AK[DRE]K[RE]GR[QDK]N[D] |
| | [QNK]E[KNR]LQ[L]R[DEK]A | E[K]LLK[EQ]AAAK[EDQ]LV | PK[ES]E[K]IAE[K]E[RHK]LK |
| | AD[ENK]K[ER]LV[A]E[RK]V | [A]E[RQ]V[A]AA[D]R[EK]AT | [ER]K[E]R[A]AK[ER]E[KTR] |
| | [A]AR[QK]R[KEN]AT[Q]S[N | [N]S[N] (SEQ ID NO: 284) | V[A]E[RKL]K[EN]L[QA]A[D] |
| | T] (SEQ ID NO: 283) | | R[KE]S[EQR]T[Q] (SEQ ID |
| | | | NO: 285) |
| DHR57_design | STEELKKVLERVRELSERAK | TDALRAVLEAVRLASEVAK | EEAKRAVEEAKRLAEEVSK |
| | ESTDPEEALKIAKEVIELALK | RVTDPDKALKIAKLVIELAL | RVTDPELSEKIRQLVKELEE |
| | AVKEDPS (SEQ ID NO: 292) | EAVKEDPS (SEQ ID NO: 293) | EAQKEDP (SEQ ID NO: 294) |
| DHR57_variants | S[D]T[DN]E[D]E[DK]LK[ER] | T[DE]D[LNT]ALR[EKQ]AVL | E[DKQ]E[LDT]AK[LA]R[EK] |
| | K[Q]VL[KIY]E[RK]R[DTK]V | [YAE]E[RKL]AVR[EKQ]LAS | AVE[K]E[LRK]AK[IEA]R[ED |
| | R[EKQ]E[HR]L[AD]S[A]E[KR | [A]E[R]V[A]AK[QER]R[K]V[I | K]LAE[KQR]E[RKQ]V[A]S[A] |
| | D]R[EQ]AK[REN]E[K]S[VIE] | L]T[N]D[N]PD[E]K[AL]AL[A | K[QER]R[NQK]V[IL]T[D]DP |
| | T[DS]D[N]P[T]E[DTN]E[KDN] | KR]K[E]I[LV]AK[ER]L[KW]V | [DS]E[NDK]L[KNS]S[AR]E[K |
| | AL[KAR]K[E]I[LV]AK[E]E | [A]I[V]E[KR]LAL[EAK]E[KD | RN]K[ER]I[LV]R[KE]Q[ERK] |
| | [KR]V[A]I[V]E[KR]L[E]AL[A | L]AV[A]K[ENR]E[QNR]D[N] | L[EKW]V[AK]K[ER]E[KRD]L |
| | EK]K[EQ]AV[A]K[RDE]E[K]D | PS (SEQ ID NO: 290) | E[RKQ]E[KR]E[LRD]AQ[KE |
| | [NK]PS (SEQ ID NO: 289) | | N]K[ER]E[QRH]D[NY]P (SEQ |
| | | | ID NO: 291) |
| DHR59_design | KTEVEKKAKEVIKEAKELA | TEVAKLALKVLEEAIELAKE | SDEARDALRRLEEAIEEAKE |
| | KELDSEEAKKVVERIKEAAE | NRSEEALKVVLEIARAALAA | NRSKESLEKVREEAKEAEQ |
| | AAKRAAEQGK (SEQ ID NO: | AQAAEEGK (SEQ ID NO: | QAEDAREG (SEQ ID NO: |
| | 298) | 299) | 300) |
| DHR59_variants | K[N]T[S]E[KT]VE[KDR]K[ED] | T[S]E[R]VAK[E]L[RK]ALK[E] | S[T]D[ER]E[V]AR[KE]D[KER] |
| | K[QET]AK[ER]E[KR]V[A]I | V[A]LE[KT]E[RQ]AIE[RK]L | ALR[EKD]R[KE]LE[KQT]E |
| | [KR]K[E]E[KRN]AK[ED]E[KR | [V]AK[EQR]E[KN]N[LAI]R[D | [KQR]AIE[RK]E[HTD]AK[EQ |
| | N]L[V]A[REV]K[RE]E[DKN] | KP]SE[KD]E[TKQ]ALK[E]VV | R]E[KQR]N[DHR]R[DKP]SK |
| | L[IA]D[KPR]SE[DKQ]E[TVL] | [A]L[A]E[AQ]I[V]AR[KE]AA | [DE]E[D]S[A]LE[KNQ]K[E]V |
| | AK[E]K[DQR]VV[A]E[K]R[E | L[KAE]A[E]AAQ[ER]AAE[K | [A]R[LKY]E[DK]E[RIW]AK[R |
| | AQ]I[V]K[R]E[KR]AAE[K]A | RQ]E[QSD]GK[N] (SEQ ID | E]E[KQN]AE[KRA]Q[EK]Q[E |
| | [E]AK[RIE]R[EKQ]AAE[KDR] | NO: 296) | KD]AE[RD]D[RKN]AR[KQS] |
| | Q[SN]GK[N] (SEQ ID NO: | | E[NR]G (SEQ ID NO: 297) |
| | 295) | | |
| DHR60_design | TDIKKKAEEIIKEAKKQGSE | DILVRAAEIVVRAQEQGSED | PTLVKAAEKVVRAQQKGSQ |
| | DAIRLAQEAKKQGT (SEQ ID | AIRLAKEASREGT (SEQ ID | DTIEKAKEESREG (SEQ ID |
| | NO: 304) | NO: 305) | NO: 306) |
| DHR60_variants | T[ND]D[TS]I[T]K[SQR]K[DE] | D[EKP]I[T]L[A]V[A]R[KDE]A | P[EQR]T[RIK]L[A]V[A]K[ER] |
| | K[ED]AE[KDN]E[RK]I[VA]I | AE[RKQ]I[VA]V[I]V[AI]R[E] | AAE[QRK]K[RE]V[I]V[AI]R |
| | [K]KE[RD]AK[QE]KQ[TEN]G | AQE[QKR]Q[EST]GSE[DRS] | [EDK]AQ[ET]Q[KRE]K[E]GSQ |
| | SE[DRS]D[TEK]AI[K]R[EK]L | D[TA]AIR[EK]L[AT]AK[REA] | [DER]D[E]T[SK]I[K]E[KR]K |
| | [AT]AQ[ERK]E[RK]AK[A]K | E[KQR]AS[A]R[E]E[QRK]GT | [RQ]AK[REN]E[KR]E[ADK]S |
| | [ERN]Q[KRE]GT[ND] (SEQ ID | [ND] (SEQ ID NO: 302) | [A]R[KE]E[KRQ]G (SEQ ID |
| | NO: 301) | | NO: 303) |
| DHR62_design | DNDEKRKRAEKALQRAQEA | NDVLRKVAEQALRIAKEAE | QDVLRKVSEQAERISKEAK |
| | EKKGDVEEAVRAAQEAVR | KQGNVEVAVKAARVAVEA | KQGNSEVSEEARKVADEAK |
| | AAKESGD (SEQ ID NO: 310) | AKQAGD (SEQ ID NO: 311) | KQTG (SEQ ID NO: 312) |
| DHR62_variants | D[SN]N[T]D[ER]E[D]K[L]R[K | N[QKT]D[E]V[LA]LR[KE]K[E | Q[KT]D[ES]V[LAS]LR[EHK] |
| | E]K[EQ]R[EK]AE[KRQ]K[ER | QR]V[A]AE[R]Q[VEA]AL[E] | K[ER]V[A]S[A]E[RQ]Q[VAE] |
| | D]AL[I]Q[KER]R[EKN]AQ[K | R[KQ]I[AV]AK[EQD]E[QL]A | AE[KQR]R[KEQ]I[AV]S[A]K |
| | ED]E[KQ]AE[RQI]K[R]K[ED | E[RQ]K[RE]Q[ED]GN[D]V[A] | [E]E[QDL]AK[ER]K[R]Q[ED] |
| | R]GD[N]V[A]E[KDR]E[RSK] | E[KRQ]V[AL]AV[A]K[EDR]A | GN[D]S[EKD]E[DQ]V[AL]S |
| | AV[A]R[KE]AA[L]Q[EK]E[R] | A[L]R[KE]V[I]AV[A]E[RD]A | [A]E[KRD]E[KQ]AR[KQE]K[E |
| | AV[A]R[EKQ]AAK[TR]E[KR] | AK[SRE]Q[EN]AGD[S] (SEQ | Q]V[I]AD[KNR]E[KRH]AK[A |
| | S[A]GD[SN] (SEQ ID NO: | ID NO: 308) | L]K[RT]Q[NE]T[A]G (SEQ ID |
| | 307) | | NO: 309) |
| DHR63_design | DPDEDRERLKEELKKIREAL | PDLAREALKEINKVIREALEI | PDLAREALEEIDKVIDEAQEI |
| | REAKEKPDPEEIKRALREVL | AKRVPDPEVIKEALRVVLEA | SERVPDEEVQREAQEVIKEA |
| | EAIRRILKLAERAGD (SEQ | IRAILKLAEQAGD (SEQ ID | DRARKKLSEQSG (SEQ ID |
| | ID NO: 316) | NO: 317) | NO: 318) |
| DHR63_variants | D[N]P[SNT]D[E]E[DK]D[A]R | PD[NEK]LAR[KEA]E[KHR]A | PD[ENK]LAR[KE]E[KR]A[VI] |
| | [EAK]E[KR]R[DEK]L[A]K[ER | [VI]L[A]K[ERD]E[AK]I[AV]N | L[AR]E[KR]E[AQK]I[AV]D[K |
| | Q]E[KRQ]E[AV]L[AIV]K[ER] | [ALE]K[RE]V[AL]I[A]R[KEQ] | E]K[ER]V[AL]I[AR]D[KER]E |
| | K[DIL]I[A]R[EK]E[KQR]AL[I | E[DIN]AL[AIQ]E[KR]I[A]AK | [IVN]AQ[EKS]E[RKN]I[A]S[A |
| | AS]R[KE]E[KDI]AK[REN]E[K | [ETQ]R[KTE]VPD[N]P[T]E[NT | KE]E[KNR]R[EKT]VPD[N]E |
| | T]K[IVT]PD[N]P[ST]E[NDQ]E | K]VIK[AER]E[AKT]ALR[EK | [PS]E[NKT]VQR[KEQ]E[QA]A |
| | [QTD]IK[ALR]R[EK]ALR[EK | N]VV[IA]L[AKQ]E[TAQ]AI[L | Q[KRE]E[KR]VI[AV]K[EDR] |
| | Q]E[IK]V[IA]L[KAQ]E[KR]AI | V]R[QEK]AI[A]L[AR]K[EQD] | E[QIK]AD[KQE]R[KEQ]AR[A |
| | [LV]R[EKD]R[KD]I]A]L[RA] | LAE[K]Q[H]AGD [N] (SEQ ID | KI]K[ET]K[ER]LS[AEK]E[KQ] |
| | K[EQ]LAE[KQR]R[KDQ]AGD | NO: 314) | Q[H]S[A]G (SEQ ID NO: 315) |
| | [N] (SEQ ID NO: 313) | | |
| DHR64_design | DPEDELKRVEKLVKEAEELL | PEVALRAVELVVRVAELLL | PEVARRAVELVKRVAELLE |
| | RQAKEKGSEEDLEKALRTA | RIAKESGSEEALERALRVAE | RIARESGSEEAKERAERVRE |
| | EEAAREAKKVLEQAEKEGD | EAARLAKRVLELAEKQGD | EARELQERVKELREREG |
| | (SEQ ID NO: 322) | (SEQ ID NO: 323) | (SEQ ID NO: 324) |
| DHR64_variants | D[S]P[S]E[DK]DE[KT]L[V]K | P[A]E[Q]V[A]AL[V]R[KE]AV | P[A]E[KRD]V[A]AR[KEH]R |
| | [ER]R[K]V[A]E[KR]K[E]L[IT | [A]E[R]LVVR[E]V[A]AE[KR] | [EKT]AV[A]E[KR]LVK[QR]R |
| | E]VK[RED]E[KQT]AE[DKQ]E | L[I]LLR[EK]I[A]AK[QEN]E[Q | [EK]V[A]AE[KDR]L[I]LE[KR] |
| | [KQ]L[KAD]L[R]R[KQE]Q[EK | DS]S[KRE]GSE[DR]E[D]ALE | R[KEQ]I[A]AR[KEN]E[QDS]S |
| | D]AK[QN]E[KR]K[E]GSE[DK] | [KQT]R[EK]AL[AE]R[EKQ]V | [EKQ]GSEE[DK]AKE[K]R[K] |
| | E[D]D[AE]LE[KDR]K[RE]AL | AE[S]E[K]AAR[K]L[Q]AK[E | AE[KQ]R[KQ]VR[EKQ]E[KD |
| | [AER]R[EKQ]T[RV]AE[AHN] | Q]R[DE]V[A]L[IA]E[DK]LAE | R]E[KQR]AR[KE]E[KR]L[E]Q |
| | E[QRK]AAR[KEN]E[R]AK[E | [QKR]K[RQ]Q[R]GD (SEQ ID | [EKR]E[KN]R[EQ]V[A]K[ED] |
| | R]K[E]V[A]L[IA]E[KDH]Q[E | NO: 320) | E[KR]LR[A]E[KR]R[K]E[Q]G |
| | KS]AE[KQ]K[ER]E[QND]GD | | (SEQ ID NO: 321) |
| | [S] (SEQ ID NO: 319) | | |
| DHR66_design | TSDDDKVREAEERVREAIER | SDAIKVAEAAARVAEAIARI | TEALKVAEKAARVAEKIARI |
| | IQRALKKRDTPDARKALEA | LEALNERDTPDARKALRAAI | LEKLNERDTPEARKKLRQAI |
| | AKKLLKVVEKAKKRGT | KLAEVVYKAAESGT (SEQ | KEAEKVYKESEQG (SEQ ID |
| | (SEQ ID NO: 328) | ID NO: 329) | NO: 330) |
| DHR66_variants | TS[DNT]D[NER]D[EQ]D[KE] | S[DTE]D[NRE]AI[AL]K[R]V | T[DER]E[DRS]AL[IA]K[EQR] |
| | K[RI]V[L]R[KED]EAE[KR]E | [L]AEAAARV[A]AE[Q]AI[A]A | V[LS]AE[K]K[EQ]AAR[KD]V |
| | [KDQ]RV[A]R[ED]E[KQR]AI | R[EK]I[A]LEAL[I]N[EKD]E[N | [A]AEK[EST]I[A]AR[DE]I[A] |
| | [EQ]E[K]R[EK]I[A]Q[KR]R[E | KR]R[NK]DT[DN]P[D]D[ES] | LE[DK]K[ER]L[I]N[KER]E[K |
| | KQ]AL[I]KK[EDN]R[NKS]D[P] | A[L]RK[EDR]AL[V]R[K]AA[I] | DR]R[NDH]D[N]T[S]P[DE]E |
| | T[SD]P[DES]D[ES]A[L]R[Q | I[V]K[EL]LAE[DK]VV[I]Y[A] | [D]A[EL]R[L]K[Q]K[EN]L[V]R |
| | K]K[REN]ALE[K]AA[I]K[QE | K[EQR]AAE[QRD]S[RQD]G | [EKQ]Q[DER]A[I]I[V]K[R]E |
| | R]K[RL]LL[AKR]K[ER]VV[I] | T (SEQ ID NO: 326) | [RDK]AE[R]K[EQ]V[I]Y[V]K |
| | E[KD]K[ERD]AK[ESQ]K[RE] | | [QER]E[KSL]S[AE]E[KQR]Q |
| | R[EQK]GT (SEQ ID NO: 325) | | [KER]G (SEQ ID NO: 327) |
| DHR67_design | TSEIDKLIKKLRQTAKEVKR | SEVAKLVWKLARTAIEVIRE | EEVAKKVWKEAYRAIEEIR |
| | EAEERKRRSTDPTVREVIER | AIERAERSTDPEVIRVILELA | KAIEKAERSTDPNEIKKILEE |
| | LAQLALDVAEEAARLIKKA | RLAAEVAKEAARLIVKATT | ARKKAEEAIERAKEIVKST |
| | TT (SEQ ID NO: 334) | (SEQ ID NO: 335) | (SEQ ID NO: 336) |
| DHR67_variants | T[ND]S[TD]E[DRT]I[LK]D[K | S[KET]E[DTK]V[LI]AK[ER]L | E[KRT]E[DN]V[LI]AK[REQ] |
| | E]K[E]LI[VK]K[ER]K[RDE]L | V[I]W[A]K[REQ]L[V]AR[AK | K[R]V[I]W[A]K[ERQ]E[LKT] |
| | [V]R[QEK]Q[KNR]T[EKQ]AK | N]T[EKR]AI[L]E[KRD]V[A]I | AY[KAQ]R[EKD]AI[L]E[KD] |
| | [D]E[KQR]V[A]K[IAE]R[KEN] | [V]R[AEK]E[DR]AI[A]E[RKQ] | E[RKN]I[V]R[AEL]K[EQR]AI |
| | E[RDQ]AE[K]E[KR]R[AL]K[I | R[AL]A[VI]E[LQA]R[EKN]S | [A]E[KD]K[DEQ]A[VI]E[RAK] |
| | QR]R[K]R[KEN]S[A]TD[NS]P | [A]TD[NS]P[DSE]E[TDN]V[L]I | R[KE]S[AET]T[NQ]D[NS]P[T |
| | [SD]T[RDN]V[L]R[A]E[KNT] | [A]R[KE]V[IL]IL[W]E[KR]L | EQ]N[ETD]E[KDN]I[A]K[ETR] |
| | V[IL]IE[KHQ]R[EK]L[AI]AQ | [AI]AR[KE]LAAE[KRS]V[I]A | K[E]I[E]L[W]E[KR]E[KNR]A |
| | [EKR]L[I]AL[KRE]D[KER]V[I] | K[IQE]E[RHK]AAR[EK]LIV | R[KE]K[E]K[IEA]AE[KR]E[K |
| | AE[KDR]E[RN]AAR[KEQ]LI | [A]KAT[KP]T[DN] (SEQ ID | R]AI[KE]E[KR]R[KET]AK[ER] |
| | K[QRE]KAT[EPQ]T[ND] | NO: 332) | E[KRQ]I[QT]V[A]K[N]S[DK] |
| | (SEQ ID NO: 331) | | T[P] (SEQ ID NO: 333) |
| DHR68_design | TPRERLEEAKERVEEIRELID | PELALRAAELLVRLIKLLIEI | PELAKRAAELLKRLIELLKEI |
| | KARKLQEQGNKEEAEKVLR | AKLLQEQGNKEEAEKVLRE | AKLLEEEGNEDEAEKVKEE |
| | EAREQIREVTRELEEIAKNS | ATELIKRVTELLEKIAKNSD | AKELEERVRELEERIRKNSD |
| | DT (SEQ ID NO: 340) | T (SEQ ID NO: 341) | (SEQ ID NO: 342) |
| DHR68_variants | TP[STN]R[EK]E[D]R[KDQ]L | P[AVN]EL[I]AL[V]RAAE[K]L | P[ATV]EL[I]AK[QE]RAAE[D |
| | [V]E[RK]E[KR]AK[ER]E[KQR] | L[I]VR[KDE]LI[V]K[ER]LLI | KR]LL[I]K[EQR]R[EK]LI[V]E |
| | R[K]VE[KDQ]E[K]I[V]R[EK] | [V]E[R]IAK[E]LLQ[AL]E[RKN] | [KR]LLK[EQR]E[RKT]IAK[E] |
| | E[KR]L[DKT]I[V]D[RKE]KA | Q[S]GNK[ST]E[D]E[D]AE[K | LLE[K]E[RQ]E[SQN]GNE[SK |
| | R[E]KLQ[AEL]E[KR]Q[SKN] | RD]K[RDS]V[A]LR[EDK]E[K | P]D[E]E[D]AE[KNQ]K[RDE] |
| | GNK[WPS]E[DKT]E[K]AE[R | RT]AT[ER]E[K]L[QAE]IK[ER] | V[A]K[EQ]E[KRD]E[KRD]AK |
| | K]K[E]V[AEQ]LR[ED]E[KQR] | R[EKN]V[A]T[AER]E[KRQ]L | [END]E[KQ]L[ADQ]E[K]E[K] |
| | AR[E]E[K]Q[DKL]IR[EKD]E | [T]LE[KNR]K[EQR]I[L]AK[R | R[KED]V[A]R[KQE]E[KQ]L[I |
| | [KR]V[A]T[AEQ]R[EKD]E[I]L | Q]NS[A]D[E]T (SEQ ID NO: | QK]E[K]E[KDQ]R[KDE]I[L]R |
| | E[KNS]E[KR]I[L]AK[E]NS[A] | 338) | [K]K[D]N[H]SD[EK] (SEQ ID |
| | D[KE]T (SEQ ID NO: 337) | | NO: 339) |
| DHR69_design | NPQEDLERAEKVVRSVEEV | PEVLLRVAELIVRLVEVVLE | PESLKRVAELIKRLVKVVDE |
| | LQRAKEAQREGDKEKVERL | LAKLAEKNGDKEQVERLIQ | LSKLAERNGDRDQVERLRQ |
| | IKEAENQIRKARELLERVVR | TAEELIREARELLERVSREIP | LAEELRREAEELEERVRRER |
| | QNPDD (SEQ ID NO: 346) | DN (SEQ ID NO: 347) | PD (SEQ ID NO: 348) |
| DHR69_variants | N[D]P[S]Q[EDK]E[DK]D[ELK] | P[WA]E[KDQ]V[AL]LL[A]R | P[WA]E[DKH]S[AL]LK[QR]R |
| | L[A]E[KR]R[KE]AE[KR]K[E | [EKQ]V[I]AE[KRQ]LI[L]V[A] | [KE]V[I]AE[DKQ]LI[L]K[ED |
| | Q]V[L]V[A]R[KE]S[KE]V[AI] | R[EDK]LV[AI]E[RKN]V[AIN] | R]R[EK]LV[ARI]K[E]V[AIN] |
| | E[KHQ]E[KR]V[ADI]L[AIV]Q | V[AI]L[AIV]E[RK]LAK[E]L[E] | V[AI]D[EK]E[KR]L[Q]S[A]K |
| | [ERK]R[KDE]AK[ER]E[KQR] | AE[QA]K[NEQ]N[EDT]GD[N] | [E]L[S]AE[KQ]R[K]N[EST]GD |
| | AQ[TS]R[KE]E[KD]GD[N]K | K[E]E[DK]Q[KET]V[A]E[RH | [N]R[EST]D[E]Q[KTD]V[A]E |
| | [E]E[DT]K[ETR]V[A]E[RKQ]R | Q]R[EKQ]LI[D]Q[EKR]T[EQ | [KRN]R[KET]LR[KEN]Q[KE]L |
| | [KE]L[R]I[T]K[E]E[KR]AE[D | D]AE[KQS]E[RK]L[DAI]I[V] | [EQT]AE[KR]E[KRQ]L[DAI]R |
| | RS]N[EKQ]Q[LKA]I[V]R[KE | R[KEQ]E[KD]AR[EKT]E[KR] | [EK]R[KE]E[KDQ]AE[KR]E |
| | Q]K[RE]AR[KTE]E[KT]L[AE | L[A]LE[DRK]R[KQE]V[A]S[A | [KQ]L[A]E[KQ]E[KR]R[ILD]V |
| | K]LE[RDQ]R[KE]V[A]V[AKR] | KR]R[KND]E[NDQ]I[RAD]PD | [A]R[KE]R[KND]E[NQT]R[A |
| | R[KN]Q[KDN]N[RAD]PD[T] | [T]N[D] (SEQ ID NO: 344) | DQ]PD[T] (SEQ ID NO: 345) |
| | D[N] (SEQ ID NO: 343) | | |
| DHR70_design | STEEKIEEARQSIKEAERSLR | TEVLIEAARLAIEVARVALK | DEVLKRAAELAKEVARVAK |
| | EGNPEKAREDVRRALELVR | VGSPETAREAVRTALELVQE | EVGSPETARQARETAERLRE |
| | ELEKLARKTGS (SEQ ID NO: | LERQARKTGS (SEQ ID NO: | ELRRNREKKG (SEQ ID NO: |
| | 352) | 353) | 354) |
| 3DHR70_variants | S[DN]T[SDN]E[DK]E[DQ]K[L | T[IDV]E[RDK]V[A]LI[ALV]E | D[TIR]E[DKQ]V[AT]LK[EQR] |
| | RT]I[ALW]E[KQ]E[DKS]AR | [KAD]AAR[EK]L[I]AI[V]E[R | R[EKT]AAE[RKD]L[I]AK[ER] |
| | [EKQ]Q[KRD]S[A]I[V]K[ER]E | KQ]V[A]AR[EKD]V[A]AL[A | E[KQR]V[A]AR[EK]V[A]AK |
| | [KQR]AE[QK]R[KE]S[ADN]L | Q]K[ERN]V[T]GS[D]P[ST]E | [QER]E[KQR]V[T]GS[D]P[SD] |
| | [AHR]R[KED]E[KQR]GN[SD] | [DQ]T[LV]AR[E]E[K]AV[ALI] | E[D]T[LSV]AR[KEQ]Q[KED] |
| | P[DKS]E[DKQ]K[STE]AR[EK] | R[EKQ]T[QLE]ALE[KNQ]L[A | AR[EKQ]E[KQR]T[LQA]AE |
| | E[KRQ]D[A]V[ALI]R[KEQ]R | I]V[A]Q[RKE]E[RD]L[IA]E[A | [KRQ]R[KDN]L[AI]R[EKD]E |
| | [KE]AL[EQ]E[KDN]L[AI]V[A] | KR]R[KE]Q[EAR]AR[EK]K[R | [KQ]E[RQA]L[IAK]R[KE]R[K |
| | R[KEQ]E[KR]L[IA]E[SAI]K | E]T[SEH]GS[DN] (SEQ ID | ED]N[EQA]R[ADN]E[KR]K[R] |
| | [ERQ]L[ERD]AR[KEQ]K[ERT] | NO: 350) | K[QRN]G (SEQ ID NO: 351) |
| | T[QRK]G[D]S[DN] (SEQ ID | | |
| | NO: 349) | | |
| DHR71_design | DPEEILERAKESLERAREASE | PELVLEAAKVALRVAELAA | PELVEEAAKVAEEVRKLAK |
| | RGDEEEFRKAAEKALELAK | KNGDKEVFKKAAESALEVA | KQGDEEVYEKARETAREVK |
| | RLVEQAKKEGD (SEQ ID | KRLVEVASKEGD (SEQ ID | EELKRVREEKG (SEQ ID NO: |
| | NO: 358) | NO: 359) | 360) |
| DHR71_variants | D[N]P[SD]E[D]E[DR]I[TVD]L | P[A]E[KQ]L[A]V[I]L[A]E[DK] | P[ALT]E[DN]L[A]V[I]E[RKQ] |
| | [AET]E[K]R[KN]AK[REQ]E | AAK[REQ]V[I]ALR[EK]V[L] | E[QKL]AAK[ERQ]V[I]AE[KR] |
| | [KR]S[AE]LE[RDK]R[KET]AR | AE[R]LAA[K]K[ER]N[KQE]G | E[RK]V[L]R[A]K[ER]LAK[E |
| | [E]E[KQ]AS[AHK]E[KN]R[K | DK[DSQ]E[DQ]VFK[QR]K[E | R]K[E]Q[KRE]GDE[DRS]E[D] |
| | DQ]GDE[DSQ]E[DQK]E[TK]F | DQ]AAE[KRD]S[TAV]ALE[K | V[L]Y[FR]E[K]K[ERQ]AR[EQ] |
| | R[KQ]K[EDR]AAE[RKQ]K[R | T]V[IL]AK[QE]R[ED]L[A]V | E[KDR]T[VA]AR[E]E[KRT]V |
| | TN]ALE[KDR]L[ITV]AK[QRE] | [A]E[KR]V[EQ]AS[KER]K[NE] | [IL]K[ETR]E[K]E[IR]L[A]K[E] |
| | R[K]L[A]V[A]E[KD]Q[ERK] | E[Q]GD[N] (SEQ ID NO: 356) | R[EKH]V[EQ]R[A]E[KT]E[K |
| | AK[ERS]K[ENQ]E[QDK]GD | | NR]K[QE]G (SEQ ID NO: 357) |
| | [N] (SEQ ID NO: 355) | | |
| DHR72_design | DSTKEKARQLAEEAKETAE | SEKAKAILLAAEAARVAKE | SEKARAILEAAERAREAKER |
| | KVGDPELIKLAEQASQEGD | VGDPELIKLALEAARRGD | GDPEQIKKARELAKRG (SEQ |
| | (SEQ ID NO: 364) | (SEQ ID NO: 365) | ID NO: 366) |
| DHR72_variants | D[N]S[TD]T[DE]K[ETS]E[DK | S[R]E[KD]K[W]AK[ER]AI[V | S[AKR]E[DKR]K[QW]AR[ED] |
| | Q]K[ERD]AR[K]Q[EDK]L[RK] | A]L[K]L[R]AAE[K]AAR[KEL] | AI[VA]L[KR]E[RK]AAE[KR] |
| | AE[KND]E[K]AK[AQI]E[KH] | V[IT]AKE[KQ]V[T]GD[NS]P | R[KET]AR[KLE]E[K]AKE[KQ] |
| | T[IVS]AE[K]K[ER]V[TA]GD | E[D]LIK[R]LAL[REQ]E[KQ]A | R[EK]GD[SN]P[S]E[DNQ]Q |
| | [NS]PE[DHN]LI[K]K[ER]L[T] | AR[KE]R[EDN]GD (SEQ ID | [KRT]IK[EQ]K[ER]AR[EKQ]E |
| | AE[KQD]Q[EKR]AS[A]Q[KD | NO: 362) | [KR]L[EK]AK[REQ]R[EK]G |
| | R]E[DQR]GD[N] (SEQ ID NO: | | (SEQ ID NO: 363) |
| | 361) | | |
| DHR73_design | DAEEEAKEAIKRAQEAIELA | AEVLALVAIALALVAIALAE | ARVLKLVAKALELVAEALK |
| | RKGNPEEARKVAEEARERA | VGNPEEAREVAERAKEIAER | KVGNPEEAREVEERAREIKE |
| | ERVREEAEKRGD (SEQ ID | VRELAEKRGD (SEQ ID NO: | RVRRLLEEKG (SEQ ID NO: |
| | NO: 370) | 371) | 372) |
| DHR73_variants | D[NS]A[SD]E[R]E[D]E[KR]A | AE[RK]V[A]LALVAIALALV | A[DIW]R[DEK]V[A]LK[EQR] |
| | KE[K]AIK[E]R[DK]AQ[K]E[R | AIALAE[QK]VGN[D]PE[D]E | LVAK[ER]ALE[K]LVAE[K]A |
| | K]AI[S]E[K]L[KDE]AR[KQE] | [S]AR[EYK]E[RK]VAE[RD]R | LK[RQ]K[QEN]VGN[D]PE[D] |
| | K[R]GN[D]PE[DK]E[SRT]AR | [EDT]AK[RYE]E[KRQ]I[LV]A | E[S]AR[EKT]E[KRS]VE[KQ]E |
| | [KE]K[E]V[TKE]AE[DR]E[DQ | E[QDR]R[E]V[A]R[EY]E[KR] | [RKQ]R[QDE]AR[EQK]E[KR] |
| | R]AR[EY]E[KR]R[ILD]AE[Q | L[EQ]AE[RQ]K[ER]R[QDN]G | I[VLT]K[QER]E[KRD]R[EDK] |
| | DK]R[EK]V[A]R[EAL]E[KR]E | D[N] (SEQ ID NO: 368) | V[A]R[KDE]R[KEQ]L[EIN]L |
| | [RKN]AE[RKQ]K[ER]R[KQ]G | | [AK]E[KRT]E[KR]K[RQN]G |
| | D[NS] (SEQ ID NO: 367) | | (SEQ ID NO: 369) |
| DHR74_design | DSEADRIIKKLQKEIKEVEQE | SEAIRIIKKLVKEITEVVREA | QEAIKRIKKLVKKIIEVVRK |
| | ARDSNDDEERELLKRLAEA | RKSTDKEEIELLIRLAEALAR | ARKSTNKKEIEKLIRKAEKL |
| | LKRAAEAVKRAQESGD | AAEAVADAAKSGD (SEQ ID | ARKAEQIAEDAKRG (SEQ |
| | (SEQ ID NO: 376) | NO: 377) | ID NO: 378) |
| DHR74_variants | D[N]S[TDN]E[DQT]AD[KEN] | S[QDE]E[QR]AI[L]R[KED]I[L] | Q[ED]E[DS]AI[L]K[EDR]R[K |
| | R[KE]I[L]I[AR]K[ED]K[RQ]L | IK[R]K[EQS]LV[A]K[EHR]E | T]IK[E]K[EQR]LV[A]K[ER]K |
| | Q[KE]K[RE]E[ALQ]IK[ED]E | [ADL]IT[IL]E[KR]V[IL]V[AI]R | [RN]II[LS]E[KQ]V[ILK]V[AI]R |
| | [KR]V[IL]E[QK]Q[EKR]E[KN | [EQK]E[R]AR[DET]K[R]S[AE | [EKQ]K[EQR]AR[EKN]K[RE |
| | Q]AR[KEN]D[KRE]S[EAR]N | Q]TDK[EPQ]E[DN]E[RK]IE[K | N]S[AEK]T[N]N[D]K[EPQ]K |
| | [T]D[N]D[SPQ]E[TD]E[KLQ]R | HR]LLI[V]R[KDL]LAEAL[A] | [ETD]E[KQR]IE[KR]K[E]L[KR] |
| | [IQ]E[KD]LLK[Q]R[KL]LAEA | ARAAEAV[A]AD[KRE]AAK | I[V]R[EKQ]K[E]AE[KQ]K[E |
| | L[A]K[QER]R[I]AAE[DKR]A | [EQ]S[TAK]GD[N] (SEQ ID | D]L[A]AR[DK]K[RE]AEQ[EN |
| | V[A]K[QDE]R[IEK]AQ[AER] | NO: 374) | R]I[AEL]AE[KR]D[RK]AK[E |
| | E[KDQ]S[TQA]GD (SEQ ID | | QR]R[KED]G (SEQ ID NO: |
| | NO: 373) | | 375) |
| DHR75_design | DSEKEKATELAERAQDVAS | SEKAKAILLAAKAVLVAVE | SEKARAILEAAREVLRAVEQ |
| | RVEEEARREGSRELIEIAREL | VYERAKRQGSDELREIAREL | YERAKRRGDDDERERAREE |
| | RERAEEASQEGD (SEQ ID | AKEALRAAQEGD (SEQ ID | AREALERAREG (SEQ ID NO: |
| | NO: 382) | NO: 383) | 384) |
| DHR75_variants | D[N]S[DNT]E[DKT]K[ES]E[D | S[APE]E[KDS]K[AR]AK[ELD] | S[APE]E[DKS]K[R]AR[EDQ] |
| | K]K[ERT]AT[KR]E[KRH]L[K | AI[V]L[AKR]L[KRE]AAK[E | AI[V]L[AKR]E[RKQ]AAR[KE |
| | ER]AE[KN]R[KE]AQ[IK]D[K | DL]AVL[KRA]V[ILT]AV[IA] | Q]E[KAR]VL[KAR]R[EKQ]A |
| | E]V[LTI]AS[KEQ]R[EKQ]V[A] | E[QR]V[A]YE[RK]R[LEK]AK | V[IA]E[QRK]Q[EKN]YE[SKA] |
| | E[LKR]E[KR]E[LR]AR[DKQ] | [RH]R[EKQ]Q[EN]GSD[ES]E | R[KET]AK[RDS]R[KE]R[KE] |
| | R[KEQ]E[TDQ]GSR[DSE]E[D | [DTK]LR[KQ]E[KNQ]IAR[EK | GD[S]D[ES]D[E]E[KDR]R[QA |
| | K]LI[EKA]E[KQN]IAR[EKQ] | Q]E[RKQ]LAK[RE]E[L]ALR | E]E[RKQ]R[KE]AR[EKN]E[R |
| | E[KQR]LR[AE]E[KR]R[LEQ] | [KEQ]AAQ[KR]E[R]GD (SEQ | DK]E[KR]AR[KE]E[KQ]ALE |
| | AE[K]E[KQR]AS[A]Q[KER]E | ID NO: 380) | [KR]R[EK]AR[KQ]E[R]G (SEQ |
| | [RK]GD[N] (SEQ ID NO: | | ID NO: 381) |
| | 379) | | |
| DHR76_design | NPELEEWIRRAKEVAKEVE | PELVEWVARAAKVAAEVIK | PELVERVARLAKKAAELIKR |
| | KVAQRAEEEGNPDLRDSAK | VAIQAEKEGNRDLFRAALEL | AIRAEKEGNRDERREALERV |
| | ELRRAVEEAIEEAKKQGN | VRAVIEAIEEAVKQGN (SEQ | REVIERIEELVRQG (SEQ ID |
| | (SEQ ID NO: 388) | ID NO: 389) | NO: 390) |
| DHR76_variants | N[DS]P[NDS]E[KDR]L[RT]E | P[WAS]E[KRD]LV[A]E[KR] | P[WA]E[KD]LV[A]E[KQR]R |
| | [KQ]E[K]W[A]I[DK]R[KD]R[E | W[A]VAR[EK]AAK[EQR]V[A] | [EKT]VAR[EKD]L[REK]AK[E |
| | K]AK[QEN]E[KRQ]V[A]AK[E | AA[V]E[KLR]V[A]I[L]K[EQ | R]K[EQ]AA[V]E[KQ]L[VAE]I |
| | ND]E[KD]V[A]E[KQR]K[E]V | R]V[LQA]AI[EL]Q[KER]A[L] | [L]K[EQ]R[KEH]AI[LE]R[EK] |
| | [LQA]AQ[KE]R[K]A[L]E[KQR] | E[QKA]K[N]E[DSN]GNR[PE | A[DL]E[QKH]K[NRQ]E[NRK] |
| | E[KRN]E[NSQ]GN[D]P[DE]D | K]D[KET]LF[ART]R[KED]A | GN[D]R[PEK]D[EK]E[KRD]R |
| | [EK]LR[AT]D[RNE]S[ALI]AK | [LV]AL[AIR]E[KR]LVR[EK]A | [AT]R[EKD]E[KR]A[N]L[AE |
| | [ENR]E[KR]LR[VIK]R[EKD] | V[I]IE[RK]AIE[KR]E[KR]AV | K]E[KR]R[KET]VR[EKD]E[K |
| | AV[I]E[QRK]E[R]AIE[KR]E | [A]K[DE]Q[K]GN[DS] (SEQ ID | N]V[I]IE[KRQ]R[TEK]IE[K]E |
| | [QR]AK[QRS]K[REN]Q[ER]G | NO: 386) | [K]L[AS]V[A]R[KDS]Q[EKR] |
| | N[DS] (SEQ ID NO: 385) | | G (SEQ ID NO: 387) |
| DHR77_design | NSDEEEAREWAERAEEAAK | SEEAEAVYWAARAVLAALE | PEEARAVYEAARDVLEALQ |
| | EALEQAKREGDEDARRVAE | ALEQAKREGDEDARRVAEE | RLEEAKRRGDEEERREAEER |
| | ELEKQAEEARRKKD (SEQ | LLRQAEEAARKKN (SEQ ID | LRQAEERARKK (SEQ ID |
| | ID NO: 394) | NO: 395) | NO: 396) |
| DHR77_variants | N[D]S[T]D[ER]E[DK]E[KDQ] | S[ARQ]E[DKS]E[LT]AE[AKQ] | P[KAE]E[KDS]E[TQD]AR[ED |
| | E[KN]AR[QK]E[KQR]W[A]A | AVY[A]W[A]A[V]AR[LEK]A | N]AVY[A]E[KRD]A[V]AR[K |
| | [V]E[DRK]R[EK]AE[KR]E[RK] | V[AI]L[A]A[L]ALE[KQR]ALE | E]D[AEK]V[AI]L[YAK]E[KR] |
| | A[L]AK[QRD]E[KR]AL[EK] | [L]Q[L]AK[Q]R[E]E[Q]GDE | ALQ[ERK]R[EK]L[Y]E[HIK]E |
| | E[K]Q[EKL]AK[QRE]R[K]E | [D]D[QK]AR[IQE]R[EK]V[L]A | [KQR]AK[ER]R[K]R[KED]GD |
| | [QR]GDE[D]D[QRE]AR[ILE]R | E[RKQ]E[RK]LLR[KE]Q[L]A | [N]E[DKQ]E[DK]E[ADK]R[K |
| | [KE]V[L]AE[KQD]E[RQ]LE[R | E[R]E[K]AA[L]R[EK]K[N]K | QI]R[KEQ]E[KRS]AE[KR]E[K |
| | L]K[ER]Q[ELR]AE[KRD]E[K] | [N]N[D] (SEQ ID NO: 392) | DR]R[EKN]LR[KE]Q[KER]AE |
| | AR[EKA]R[EK]K[N]K[NHQ] | | [KR]E[KR]R[AKN]A[Q]R[ED |
| | D[NS] (SEQ ID NO: 391) | | K]K[NR]K[NEH] (SEQ ID NO: |
| | | | 393) |
| DHR79_design | SSDEEEARELIERAKEAAER | SDVNEALKLIVEAIEAAVRA | EEVNEALKKIVKAIQEAVES |
| | AQEAAERTGDPRVRELARE | LEAAERTGDPEVRELARELV | LREAEESGDPEKREKARERV |
| | LKRLAQEAAEEVKRDPSS | RLAVEAAEEVQRNPSS (SEQ | REAVERAEEVQRDPS (SEQ |
| | (SEQ ID NO: 400) | ID NO: 401) | ID NO: 402) |
| DHR79_variants | S[ND]S[DTN]D[E]E[DK]E[KD] | S[DKE]D[N]V[A]N[RV]E[RK] | E[DKQ]E[DS]V[AS]N[VA]E[R |
| | E[KRT]AR[EK]E[KR]L[RAE] | AL[A]K[ED]L[R]I[V]V[IL]E[K | DK]AL[A]K[E]K[ER]I[V]V[IL] |
| | I[TK]E[RK]R[KE]AK[EQ]E[K | R]AIE[K]AAVR[EAK]ALE[K] | K[RQ]A[L]IQ[EKD]E[DK]AV |
| | R]AA[S]E[KRD]R[EKL]AQ[E | AAE[IKN]R[KQ]T[V]GDPE[K | E[RKQ]S[A]LR[EKQ]E[KNR] |
| | KN]E[QR]AAE[KNR]R[EKN] | RN]V[A]R[I]E[K]LAR[AV]E | AE[NKQ]E[RDK]S[KTE]GD |
| | T[A]GDPR[KNT]V[A]R[IK]E | [KR]LVR[EQ]LAVE[RK]AAE | [N]PE[NQ]K[EQ]R[IKQ]E[K]K |
| | [K]LAR[KE]E[KR]LK[SRV]R | [K]E[RKN]VQ[WLD]R[EK]N | [RE]AR[AV]E[KR]R[EKQ]VR |
| | [ED]LAQ[EKR]E[RKN]AAE[K | [D]PS[RK]S[DN] (SEQ ID NO: | [E]E[KR]AVE[RK]R[KET]AE |
| | R]E[QRD]VK[QE]R[K]DPS[R | 398) | [QK]E[K]V[I]Q[HLA]R[KN]DP |
| | T]S[ND] (SEQ ID NO: 397) | | S[NRT] (SEQ ID NO: 399) |
| DHR80_design | NSEELERESEEAERRLQEAR | SEEAERASEKAQRVLEEAR | KEEAERAYEDARRVEEEAR |
| | KRSEEARERGDLKELAEALI | KVSEEAREQGDDEVLALALI | KVKESAEEQGDSEVKRLAE |
| | EEARAVQELARVASERGN | AIALAVLALAEVASSRGN | EAEQLAREARRHVQETRG |
| | (SEQ ID NO: 406) | (SEQ ID NO: 407) | (SEQ ID NO: 408) |
| DHR80_variants | N[SD]S[TD]E[DK]E[DKQ]L[A | S[RDK]E[DKQ]E[TLA]AE[DK | K[QSR]E[DKS]E[KTA]AE[KD |
| | D]E[KRQ]R[KE]E[RN]S[EAH] | Q]R[EKQ]AS[AEK]E[KR]K[R | R]R[EK]AY[AEK]E[KRQ]D[K |
| | E[KR]E[KD]AE[KQR]R[KE]R | EW]AQ[EKR]R[KQE]VL[YAE] | ER]AR[EKQ]R[EKQ]VE[KYA] |
| | [EKD]L[YAE]Q[REK]E[KR]A | E[KRQ]E[QDK]AR[EKQ]K[E] | E[KR]E[RKS]AR[EKS]K[E]V |
| | R[KE]K[E]R[EK]S[A]E[K]E[K | V[I]S[A]E[KRD]E[KRQ]AR[E | [I]K[AR]E[RK]S[EQR]AE[KD |
| | R]AR[KEQ]E[KR]R[KQT]GD | K]E[K]Q[KEN]GD[N]D[LYE] | Q]E[KR]Q[KN]GD[N]S[DEQ] |
| | LK[REQ]E[TAK]L[AEK]AE[K] | E[RQK]V[A]L[A]ALALIAI[A | E[K]V[A]K[LYA]R[KND]LAE |
| | ALIE[KRI]E[RIA]AR[QKD]A | RQ]AL[Q]AV[A]L[AV]AL[IK | [KNQ]E[TKR]AE[IAR]Q[EKN] |
| | V[A]Q[KAE]E[KIR]L[IAK]AR | A]AE[ILV]V[A]AS[AEK]S[A] | L[K]AR[EKD]E[RK]AR[IKA] |
| | [EK]V[A]AS[KAE]E[RKD]R | R[EKS]GN[DS] (SEQ ID NO: | R[KQE]H[ILQ]V[A]Q[KAD]E |
| | [KEA]GN[SD] (SEQ ID NO: | 404) | [KRD]T[SA]R[KQS]G (SEQ ID |
| | 403) | | NO: 405) |
| DHR82_design | NDEEVQEAVERAEELREEA | DEAVETAVRLARELKKVAE | EEAVETAKRLAEELRKVAE |
| | EELIKKARKTGDPELLRKAL | ELQERAKKTGDPELLKLAL | LLEERAKETGDPELQELAKR |
| | EALEEAVRAVEEAIKRNPDN | RALEVAVRAVELAIKSNPD | AKEVADRARELAKKSNPN |
| | (SEQ ID NO: 412) | N (SEQ ID NO: 413) | (SEQ ID NO: 414) |
| DHR82_variants | N[D]D[TS]E[DR]E[DR]V[AD] | D[EKS]E[DK]AV[A]E[KNR]T | E[KQR]E[DRK]AV[A]E[KDR] |
| | Q[KE]E[KRQ]A[KL]V[ANQ]E | [AIL]A[L]V[ANQ]R[EDK]L[I | T[AIL]A[L]K[QEA]R[EKS]L[I |
| | [RK]R[KDE]AE[QRD]E[RQK] | AV]AR[EKQ]E[LDR]L[A]K[A | AV]AE[KQR]E[LRA]L[A]R[K |
| | L[A]R[AEI]E[KR]E[KD]AE[R | QI]K[ER]V[AI]AE[KQR]E[DK | EQ]K[RES]V[AI]AE[KR]L[DE |
| | KQ]E[K]L[AW]I[AEQ]K[DER] | L]L[A]Q[IAE]E[KQR]R[LEI]A | I]L[A]E[K]E[KRD]R[LIQ]AK |
| | K[E]AR[KEQ]K[E]T[EK]G[N] | K[REQ]K[RD]T[E]GD[NTS]P | [QER]E[KR]T[HQ]G[N]D[TNS] |
| | D[TNS]P[T]E[DTQ]L[AK]LR | [T]E[QRT]L[A]LK[RDE]L[EK | P[ERS]E[TDR]L[A]Q[EK]E[K |
| | [KDE]K[E]AL[IVA]E[R]A[KR | W]AL[IVA]R[EK]AL[V]E[KI] | N]L[AKI]AK[EQ]R[EKD]AK |
| | W]L[V]E[IKR]E[KR]AV[AI]R | V[AL]AV[AI]R[KEQ]AV[A]E | [ER]E[KR]V[AL]AD[KNR]R[E |
| | [EK]A[L]V[A]E[AKR]E[QKR] | [A]L[AEI]AI[L]K[RE]S[A]N[D | K]AR[EKQ]E[KQ]L[AEI]AK |
| | AI[L]K[RD]R[DQE]N[DER]P | RE]PD[NSE]N[D] (SEQ ID | [RQ]K[RDE]S[A]N[DRS]PN[D |
| | D[RGN]N[D] (SEQ ID NO: | NO: 410) | ST] (SEQ ID NO: 411) |
| | 409) | | |

In another embodiment, the polypeptide comprises or consists of the amino acid sequence selected from the group consisting of:
(A) SEQ ID NO:4-[SEQ ID NO:5]_{(0 or 2-19)}-SEQ ID NO:6;
(B) SEQ ID NO:10-[SEQ ID NO:11]_{(0 or 2-19)}-SEQ ID NO:12;
(C) SEQ ID NO:16-[SEQ ID NO:17]_{(0 or 2-19)}-SEQ ID NO:18;
(D) SEQ ID NO:22-[SEQ ID NO:23]_{(0 or 2-19)}-SEQ ID NO:24;
(E) SEQ ID NO:28-[SEQ ID NO:29]_{(0 or 2-19)}-SEQ ID NO:30;
(F) SEQ ID NO:34-[SEQ ID NO:35]_{(0 or 2-19)}-SEQ ID NO:36;
(G) SEQ ID NO:40-[SEQ ID NO:41]_{(0 or 2-19)}-SEQ ID NO:42;
(H) SEQ ID NO:46-[SEQ ID NO:47]_{(0 or 2-19)}-SEQ ID NO:48;
(I) SEQ ID NO:52-[SEQ ID NO:53]_{(0 or 2-19)}-SEQ ID NO:54;
(J) SEQ ID NO:58-[SEQ ID NO:59]_{(0 or 2-19)}-SEQ ID NO:60;
(K) SEQ ID NO:64-[SEQ ID NO:65]_{(0 or 2-19)}-SEQ ID NO:66;
(L) SEQ ID NO:70-[SEQ ID NO:71]_{(0 or 2-19)}-SEQ ID NO:72;
(M) SEQ ID NO:76-[SEQ ID NO:77]_{(0 or 2-19)}-SEQ ID NO:78;
(N) SEQ ID NO:82-[SEQ ID NO:83]_{(0 or 2-19)}-SEQ ID NO:84;
(O) SEQ ID NO:88-[SEQ ID NO:89]_{(0 or 2-19)}-SEQ ID NO:90;
(P) SEQ ID NO:94-[SEQ ID NO:95]_{(0 or 2-19)}-SEQ ID NO:96;
(Q) SEQ ID NO:100-[SEQ ID NO:101]_{(0 or 2-19)}-SEQ ID NO:102;
(R) SEQ ID NO:106-[SEQ ID NO:107]_{(0 or 2-19)}-SEQ ID NO:108;
(S) SEQ ID NO:112-[SEQ ID NO:113]_{(0 or 2-19)}-SEQ ID NO:114;
(T) SEQ ID NO:118-[SEQ ID NO:119]_{(0 or 2-19)}-SEQ ID NO:120;
(U) SEQ ID NO:124-[SEQ ID NO:125]_{(0 or 2-19)}-SEQ ID NO:126;
(V) SEQ ID NO:130-[SEQ ID NO:131]_{(0 or 2-19)}-SEQ ID NO:132;
(W) SEQ ID NO:136-[SEQ ID NO:137]_{(0 or 2-19)}-SEQ ID NO:138;
(X) SEQ ID NO:142-[SEQ ID NO:143]_{(0 or 2-19)}-SEQ ID NO:144;
(Y) SEQ ID NO:148-[SEQ ID NO:149]_{(0 or 2-19)}-SEQ ID NO:150;
(Z) SEQ ID NO:154-[SEQ ID NO:155]_{(0 or 2-19)}-SEQ ID NO:156;
(AA) SEQ ID NO:160-[SEQ ID NO:161]_{(0 or 2-19)}-SEQ ID NO:162;
(BB) SEQ ID NO:166-[SEQ ID NO:167]_{(0 or 2-19)}-SEQ ID NO:168;
(CC) SEQ ID NO:172-[SEQ ID NO:173]_{(0 or 2-19)}-SEQ ID NO:174;
(DD) SEQ ID NO:178-[SEQ ID NO:179]_{(0 or 2-19)}-SEQ ID NO:180;
(EE) SEQ ID NO:184-[SEQ ID NO:185]_{(0 or 2-19)}-SEQ ID NO:186;
(FF) SEQ ID NO:190-[SEQ ID NO:191]_{(0 or 2-19)}-SEQ ID NO:192;
(GG) SEQ ID NO:196-[SEQ ID NO:197]_{(0 or 2-19)}-SEQ ID NO:198;
(HH) SEQ ID NO:202-[SEQ ID NO:203]_{(0 or 2-19)}-SEQ ID NO:204;
(II) SEQ ID NO:208-[SEQ ID NO:209]_{(0 or 2-19)}-SEQ ID NO:210;
(JJ) SEQ ID NO:214-[SEQ ID NO:215]_{(0 or 2-19)}-SEQ ID NO:216;
(KK) SEQ ID NO:220-[SEQ ID NO:221]_{(0 or 2-19)}-SEQ ID NO:222;
(LL) SEQ ID NO:226-[SEQ ID NO:227]_{(0 or 2-19)}-SEQ ID NO:228;
(MM) SEQ ID NO:232-[SEQ ID NO:233]_{(0 or 2-19)}-SEQ ID NO:234;
(NN) SEQ ID NO:238-[SEQ ID NO:239]_{(0 or 2-19)}-SEQ ID NO:240;
(OO) SEQ ID NO:244-[SEQ ID NO:245]_{(0 or 2-19)}-SEQ ID NO:246;
(PP) SEQ ID NO:250-[SEQ ID NO:251]_{(0 or 2-19)}-SEQ ID NO:252;
(QQ) SEQ ID NO:256-[SEQ ID NO:257]_{(0 or 2-19)}-SEQ ID NO:258;
(RR) SEQ ID NO:262-[SEQ ID NO:263]_{(0 or 2-19)}-SEQ ID NO:264;
(SS) SEQ ID NO:268-[SEQ ID NO:269]_{(0 or 2-19)}-SEQ ID NO:270;
(TT) SEQ ID NO:274-[SEQ ID NO:275]_{(0 or 2-19)}-SEQ ID NO:276;
(UU) SEQ ID NO:280-[SEQ ID NO:281]_{(0 or 2-19)}-SEQ ID NO:282;
(VV) SEQ ID NO:286-[SEQ ID NO:287]_{(0 or 2-19)}-SEQ ID NO:288;
(WW) SEQ ID NO:292-[SEQ ID NO:293]_{(0 or 2-19)}-SEQ ID NO:294;
(XX) SEQ ID NO:298-[SEQ ID NO:299]_{(0 or 2-19)}-SEQ ID NO:300;
(YY) SEQ ID NO:304-[SEQ ID NO:305]_{(0 or 2-19)}-SEQ ID NO:306;
(ZZ) SEQ ID NO:310-[SEQ ID NO:311]_{(0 or 2-19)}-SEQ ID NO:312;
(AAA) SEQ ID NO:316-[SEQ ID NO:317]_{(0 or 2-19)}-SEQ ID NO:318;
(BBB) SEQ ID NO:322-[SEQ ID NO:323]_{(0 or 2-19)}-SEQ ID NO:324;
(CCC) SEQ ID NO:328-[SEQ ID NO:329]_{(0 or 2-19)}-SEQ ID NO:330;
(DDD) SEQ ID NO:334-[SEQ ID NO:335]_{(0 or 2-19)}-SEQ ID NO:336;
(EEE) SEQ ID NO:340-[SEQ ID NO:341]_{(0 or 2-19)}-SEQ ID NO:342;
(FFF) SEQ ID NO:346-[SEQ ID NO:347]_{(0 or 2-19)}-SEQ ID NO:348;
(GGG) SEQ ID NO:352-[SEQ ID NO:353]_{(0 or 2-19)}-SEQ ID NO:354;
(HHH) SEQ ID NO:358-[SEQ ID NO:359]_{(0 or 2-19)}-SEQ ID NO:360;
(III) SEQ ID NO:364-[SEQ ID NO:365]_{(0 or 2-19)}-SEQ ID NO:366;
(JJJ) SEQ ID NO:370-[SEQ ID NO:371]_{(0 or 2-19)}-SEQ ID NO:372;
(KKK) SEQ ID NO:376-[SEQ ID NO:377]_{(0 or 2-19)}-SEQ ID NO:378;
(LLL) SEQ ID NO:382-[SEQ ID NO:383]_{(0 or 2-19)}-SEQ ID NO:384;
(MMM) SEQ ID NO:388-[SEQ ID NO:389]_{(0 or 2-19)}-SEQ ID NO:390;
(NNN) SEQ ID NO:394-[SEQ ID NO:395]_{(0 or 2-19)}-SEQ ID NO:396;
(OOO) SEQ ID NO:400-[SEQ ID NO:401]_{(0 or 2-19)}-SEQ ID NO:402;
(PPP) SEQ ID NO:406-[SEQ ID NO:407]_{(0 or 2-19)}-SEQ ID NO:408; and
(QQQ) SEQ ID NO:412-[SEQ ID NO:413]_{(0 or 2-19)}-SEQ ID NO:414;
wherein the domain in brackets is an optional internal domain.

The polypeptides of this embodiment include 2 or 3 domains (as described above), and are represented in Table 1 above, reflected in each row showing listed as “DHRx_design” (where x is replaced by a specific number in the table).

In one embodiment of any aspect or embodiment of the polypeptides, the internal domain is absent. In certain alternative embodiments, the polypeptides according to this aspect further comprise at least one of an Ncap domain coupled to the N-terminus of the at least two Internal domains and a Ccap domain coupled to the C-terminus of the at least two Internal domains. In certain embodiments, the optional internal domain is present in 2-19 copies. In certain specific embodiments, the optional internal domain is present in 2-3 copies.

In another aspect, the invention provides polypeptides comprising or consisting of a polypeptide having at least 50% identity over its length with a polypeptide having the amino acid sequence selected from the group consisting of SEQ ID NO: 415-497 (see Table 2). The polypeptides of this aspect of the invention represent novel repeat proteins with precisely specified geometries identified using the methods of the invention, opening up a wide array of new possibilities for biomolecular engineering. In various embodiments, the polypeptides comprise or consist of a polypeptide having at least 55%, 60%, 65%, 70%, 75%, 80%, 85%, 90%, 95%, 96%, 97%, 98%, 99%, or 100% identity over its length with a polypeptide having the amino acid sequence selected from the group consisting of SEQ ID NO: 415-497.

TABLE 2

| Name | Sequence |
|---|---|
| DHR1 | GCDQVAKDASSTIREVIEKNPNYSEKVADVAAKIVKKIIEGNPNGC |
| | DCVAKAASSIIRAVIEKNPNYSEVVADVAAAIVKAIIEGNPNGCDCVA |
| | KAASSIIRAVIEKNPNYSEVVADVAAAIVKAIIEGNPNGRDCVRKAAS |
| | SIIRAVQEKNPNYSEVVEDVKRAIEKAIKEGNPN (SEQ ID NO: 415) |
| DHR2 | SDADEAAKEANKAENKARNRNDDEAAKAVKLIKEAIERAKKRNESD |
| | AVEAAKEAAKALNKALNRNDDEAAKAVALIAEAIIRALKRNESDAVE |
| | AAKEAAKALNKALNRNDDEAAKAVALIAEAIIRALKRNESDAVEKAK |
| | EAAKNLNKALNRNDDEQAKHVAKQAENIIRALKRNES (SEQ ID NO: |
| | 416) |
| DHR3 | SSEDTVRKIAQKCSEAIRESNDCEEAARKCAKTISEAIRESNSSELAVRI |
| | IAQVCSEAIRESNDCECAARICAKIISEAIRESNSSELAVRIIAQVCSEAIR |
| | ESNDCECAARICAKIISEAIRESNSSELAKRIIKQVCSEAKRESNDTECA |
| | KRICTKIKSEAKRESNS (SEQ ID NO: 417) |
| DHR4 | SYEDECEEKARRVAEKVERLKRSGTSEDEIAEEVAREISEVIRTLKESG |
| | SSYEVICECVARIVAEIVEALKRSGTSEDEIAEIVARVISEVIRTLKESGS |
| | SYEVICECVARIVAEIVEALKRSGTSEDEIAEIVARVISEVIRTLKESGSS |
| | YEVIKECVQRIVEEIVEALKRSGTSEDEINEIVRRVKSEVERTLKESGSS |
| | (SEQ ID NO: 418) |
| DHR5 | SSEKEELRERLVKICVENAKRKGDDTEEAREAAREAFELVREAAERA |
| | GIDSSEVLELAIRLIKECVENAQREGYDISEACRAAAEAFKRVAEAAK |
| | RAGITSSEVLELAIRLIKECVENAQREGYDISEACRAAAEAFKRVAEAA |
| | KRAGITSSETLKRAIEEIRKRVEEAQREGNDISEACRQAAEEFRKKAEE |
| | LKRRGD (SEQ ID NO: 419) |
| DHR6 | SEEKEEALKKVREAAKKLGSSDEEARKCFEEAREWAERTGSSAYEAA |
| | EALFKVLEAAYKLGSSAEEACECFNQAAEWAERTGSGAYEAAEALFK |
| | VLEAAYKLGSSAEEACECFNQAAEWAERTGSGAYEAAERLFEELERA |
| | YEEGSSAEEACEEFNKKEEEAHRKGKK (SEQ ID NO: 420) |
| DHR7 | STKEDARSTCEKAARKAAESNDEEVAKQAAKDCLEVAKQAGMPTKE |
| | AARSFCEAAARAAAESNDEEVAKIAAKACLEVAKQAGMPTKEAARS |
| | FCEAAARAAAESNDEEVAKIAAKACLEVAKQAGMPTKEAARSFCEA |
| | AKRAAKESNDEEVEKIAKKACKEVAKQAGMP (SEQ ID NO: 421) |
| DHR8 | SDEMKKVMEALKKAVELAKKNNDDEVAREIERAAKEIVEALRENNS |
| | DEMAKVMLALAKAVLLAAKNNDDEVAREIARAAAEIVEALRENNSD |
| | EMAKVMLALAKAVLLAAKNNDDEVAREIARAAAEIVEALRENNSDE |
| | MAKKMLELAKRVLDAAKNNDDETAREIARQAAEEVEADRENNS |
| | (SEQ ID NO: 422) |
| DHR9 | SYEDEAEEKARRVAEKVERLKRSGTSEDEIAEEVAREISEVIRTLKESG |
| | SSYEVIAEIVARIVAEIVEALKRSGTSEDEIAEIVARVISEVIRTLKESGSS |
| | YEVIAEIVARIVAEIVEALKRSGTSEDEIAEIVARVISEVIRTLKESGSSY |
| | EVIKEIVQRIVEEIVEALKRSGTSEDEINEIVRRVKSEVERTLKESGSS |
| | (SEQ ID NO: 423) |
| DHR10 | SSEKEELRERLVKIVVENAKRKGDDTEEAREAAREAFELVREAAERA |
| | GIDSSEVLELAIRLIKEVVENAQREGYDISEAARAAAEAFKRVAEAAK |
| | RAGITSSEVLELAIRLIKEVVENAQREGYDISEAARAAAEAFKRVAEA |
| | AKRAGITSSETLKRAIEEIRKRVEEAQREGNDISEAARQAAEEFRKKAE |
| | ELKRRGD (SEQ ID NO: 424) |
| DHR11 | SDADEAAKEANKAENKARNRNDDEAAKAVKLCKEAIERAKKRNESD |
| | AVEAAKEAAKALNKALNRNDDEAAKAVALCCEAIIRALKRNESDAV |
| | EAAKEAAKALNKALNRNDDEAAKAVALCCEAIIRALKRNESDAVEK |
| | AKEAAKNLNKALNRNDDEQAKHVAKQCENTIRALKRNES (SEQ ID |
| | NO: 425) |
| DHR12 | DDEEQCREIAEKAKQTYTDDEEIARIIAEAARQTTTDDEEICRCIAEAA |
| | KQTYTDDEEIARIIAYAARQTTTDDEEICRCIAEAAKQTYTDDEEIARII |
| | AYAARQTTTDDEEIERCIEEAAKQTYTDDEEIERIKEYARRQTTTD |
| | (SEQ ID NO: 426) |
| DHR13 | NAEDKAREVLKELKDEGSPEEEAARQVLKDLNREGSNAEDAARAVL |
| | KALKDEGSPEEEAARAVLKALNREGSNAEDAARAVLKALKDEGSPEE |
| | EAARAVLKALNREGSNEEDASRAVLKALKDEGSPEEEARRAVEKALN |
| | REGSN (SEQ ID NO: 427) |
| DHR14 | DSEEVNERVKQLAEKAKEATDKEEVIEIVKELAELAKQSTDSELVNEI |
| | VKQLAEVAKEATDKELVIYIVKILAELAKQSTDSELVNEIVKQLAEVA |
| | KEATDKEL VIYIVKILAELAKQSTDSELVNEIVKQLEEVAKEATDKEL |
| | VEHIEKILEELKKQSTD (SEQ ID NO: 428) |
| DHR15 | NDERQKQREEVRKLAEELASKATDEELIKEIKKCAQLAEELASRSTND |
| | ELIKQILEVAKLAFELASKATDEELIKEILKCCQLAFELASRSTNDELIK |
| | QILEVAKLAFELASKATDEELIKEILKCCQLAFELASRSTNDEEIKQILE |
| | TAKEAFERASKATDEEEIKEILKKCQEKFEKKSRSTN (SEQ ID NO: 429) |
| DHR16 | NDKAKEAEELLRKALEKAEKENDETAIRCVELLKEALERAKKNNNDK |
| | AIEAVELLAKALEKALKENDETAIRCVCLLAEALLRALKNNNDKAIEA |
| | VELLAKALEKALKENDETAIRCVCLLAEALLRALKNNNDKAIEEVER |
| | LAKELEKALKENDETKIREVCERAEELLRRLKNNN (SEQ ID NO: 430) |
| DHR17 | SSEDAREKIEQLCREAKEIAERAKQQNSQEEAREAIEKLLRIAKRIAEL |
| | AKQANQSEVAREAIECLCRIAKLIAELAKQANSQEVAREAIEALLRIAK |
| | LIAELAKQANQSEVAREAIECLCRIAKLIAELAKQANSQEVAREAIEAL |
| | LRIAKLIAELAKQANQSEVAREAIECLSRIAKLIEELAKQANSQEVKRE |
| | AQEALDRIQKLIEELQKQANQ (SEQ ID NO: 431) |
| DHR18 | DIEKLCKKAESEAREARSKAEELRQRHPDSQAARDAQKLASQAEEAV |
| | KLACELAQEHPNADIAKLCIKAASEAAEAASKAAELAQRHPDSQAAR |
| | DAIKLASQAAEAVKLACELAQEHPNADIAKLCIKAASEAAEAASKAA |
| | ELAQRHPDSQAARDAIKLASQAAEAVKLACELAQEHPNADIAKKCIK |
| | AASEAAEEASKAAEEAQRHPDSQKARDEIKEASQKAEEVKERCERAQ |
| | EHPNA (SEQ ID NO: 432) |
| DHR19 | DEIEKVREEAEKLKKKTDDEDVLEVAREAIRAAKEATSDEILKVIKEA |
| | LKLAKKTTDKDVLEVAREAIRAAEEATDDEILKVIKEALKLAKKTTD |
| | KDVLEVAREAIRAAEEATDEEILKEIKEALKKAKETTDTEELEKAREQI |
| | RKAEESTD (SEQ ID NO: 433) |
| DHR20 | SDIEEIRQLAEELRKKSDNEEVRKLAQEAAELAKRSTDSDVLEIVKDA |
| | LELAKQSTNEEVIKLALKAAVLAAKSTDSDVLEIVKDALELAKQSTNE |
| | EVIKLALKAAVLAAKSTDEEVLEEVKEALRRAKESTDEEEIKEELRKA |
| | VEEAESTD (SEQ ID NO: 434) |
| DHR21 | SEKEKVEELAQRIREQLPDTELAREAQELADEARKSDDSEALKVVYL |
| | ALRIVQQLPDTELAREALELAKEAVKSTDSEALKVVYLALRIVQQLPD |
| | TELAREALELAKEAVKSTDQEALKSVYEALQRVQDKPNTEEARESLE |
| | RAKEDVKSTD (SEQ ID NO: 435) |
| DHR22 | DDAEELRERARDLLRKNGSSEEEIKKVDEELEKIVRKADSDDAVKLA |
| | VKAAALLAENGSSAEEIVKVLEELLKIVEKADSDDAVKLAVKAAALL |
| | AENGSSAEEIVKVLEELLKIVEKADSEEEVKDAVREAAELAERGSSAE |
| | EIRKQLKDRLRKVEESDS (SEQ ID NO: 436) |
| DHR23 | SDSEKLAKRVLKELKRRGTSDEELERMKRELEKIIKSATSSDAMRLAL |
| | RVVLELVRRGTSSEILEKMMRMLIKIIQSATSSDAMRLALRVVLELVR |
| | RGTSSEILEKMMRMLIKIIQSATSDDQMREALRQVLEEVRKGTSSEQL |
| | ERSMRKLIKEIKKRTS (SEQ ID NO: 437) |
| DHR24 | SEAEELARRAAKEAKELCKRSTDEELCKELKKLAELLKELAERYPDSE |
| | AAKLALKAALEAIELCKQSTDEELCEELVKLAQKLIELAKRYPDSEAA |
| | KLALKAALEAIELCKQSTDEELCEELVKLAQKLIELAKRYPDSEEAKR |
| | ALKEAKELIEQCKESTDEDECRELVKRAEELIREAKENPD (SEQ ID |
| | NO: 438) |
| DHR25 | DERDKVRELIDRVEKELKREGTSEELIEEIRKVLKKAKEAADSDDDEAI |
| | KVAKEIVRVILELVREGTSSELIEEILKVLSLAAEAAKSTDDEAIKVAK |
| | EIVRVILELVREGTSSELIEEILKVLSLAAEAAKSTDEEAIKKAKEIVRRI |
| | LELTREGTSEEEIREELKELRKKAQKAKSPE (SEQ ID NO: 439) |
| DHR26 | DECERLRQEVEKAEKELEKLAKQSTDEEVRQIAREVAKQLRRLAEEA |
| | CRSNSDECLRLASEVVKAVQELVKLAEQATDEEVIRVALEVARELIRL |
| | AQEACRSNDDECLRLASEVVKAVQELVKLAEQATDEEVIRVALEVAR |
| | ELIRLAQEACRSNDEECLREASEVVKEVQELVKEAEKSTDEEEIRELLQ |
| | RAEERIREAQERCREGD (SEQ ID NO: 440) |
| DHR27 | TRQKEQLDEVLEEIQRLAEEARKLMTDEEEAKKIQEEAERAKEMLRR |
| | AVEKVTDNEVIEKLLEVVKEIIRLAEEAMKKMTDEEEAAKIAKEALEA |
| | IKMLARAVEEVTDNEVIEKLLEVVKEIIRLAEEAMKKMTDEEEAAKIA |
| | KEALEAIKMLARAVEEVTDKERIEQLLREVKEEIRRAEEESRKETDDE |
| | EAAKRAREALRRIRERAREVEEDKS (SEQ ID NO: 441) |
| DHR28 | DEEVQRIREEVRRAIEEVRESLERNDSEEAEELAREALERVAEEVKESI |
| | KERPDRDLAIEAIRALVRLAIEIVRLALEQNDSELAREVAEEALRAVAE |
| | VVKEAIRQRGDRDLAIEAIRALVRLAIEIVRLALEQNDSELAREVAEEA |
| | LRAVAEVVKEAIRQRGDRELAKEAIRALRRLAEEIRRLAEEQNDDELA |
| | REVEELAREAIEEVRKELERQRPGR (SEQ ID NO: 442) |
| DHR29 | SEVEESAQEVEKRAQEVREEAERRGTSQEVLDEIKRVVDEARQLAQR |
| | AKESDDSEVAESALQVVREALKVVLSALERGTSEEVLKEILRVVSEAI |
| | KLALEAIKSSDSEVAESALQVVREALKVVLSALERGTSEEVLKEILRV |
| | VSEAIKLALEAIKSSDSETARRALEKVRESLKEVLEQLERGTSEEELRE |
| | SLREVSENIRKALEEIKSPD (SEQ ID NO: 443) |
| DHR30 | STVKELLDRARELMRELAERASEQGSDEEEARKLLEDLEQLVQEIRRE |
| | LEETGTSSEVIRLIAKAIMLMAELALRAAEQGSDAEEAMKLLKDLLRL |
| | VLEILRELRETGTDSEVIRLIAKAIMLMAELALRAAEQGSDAEEAMKL |
| | LKDLLRLVLEILRELRETGTDKEEIRKVAEEIMRRAKTALDEARQGSD |
| | AEEAMKRLKEQLRRILERLREEREKGTD (SEQ ID NO: 444) |
| DHR31 | DSYTERARKAVKRYVKEEGGSEEEAEREAEKVREEIRKKASDSYLIQA |
| | AAAVVAYVIEEGGSPEEAVKIAEEVVRRIKEKADDSYLIQAAAAVVA |
| | YVIEEGGSPEEAVKIAEEVVRRIKEKADDRELIRRAAERVAEVIERGGS |
| | PEEAVKEAEKEVKKQKEESD (SEQ ID NO: 445) |
| DHR32 | SIQEKAKQSVIRKVKEEGGSEEEARERAKEVEERLKKEADDSTLVRAA |
| | AAVVLYVLEKGGSTEEAVQRAREVIERLKKEASDSTLVRAAAAVVLY |
| | VLEKGGSTEEAVQRAREVIERLKKEASDEELIREAAKEVLKVLEEGGS |
| | VEEAVERARERIEELQKRSDD (SEQ ID NO: 446) |
| DHR33 | SETEEVKKLVEEKVKKEGGSPEEAKETAKEVTEELKEESQDSTLLKVA |
| | ALVASAVLKEGGSPEEAAETAKEVVKELRKSASDSTLLKVAALVASA |
| | VLKEGGSPEEAAETAKEVVKELRKSASDEELLKEAARQAEESLRQGK |
| | SPEEAAEEAKKEVKKLKEKSQD (SEQ ID NO: 447) |
| DHR34 | SETEEVKKLCEEKVKKEGGSPEEAKETAKEVTEELKEESQDSTLLKVA |
| | ALCASAVLKEGGSCEEAAETAKEVVKELRKSASDSTLLKVAALCASA |
| | VLKEGGSCEEAAETAKEVVKELRKSASDEELLKEAARQAEESLRQGK |
| | SCEEAAEEAKKEVKKLKEKSQD (SEQ ID NO: 448) |
| DHR35 | SEEDEVAKQASRYAKEQGGDPEKSREEAEKALEEVKKQATSSEALQV |
| | ALEAARYASEEGEDPAEALKEAARALEEVRRSATSSEALQVALEAAR |
| | YASEEGEDPAEALKEAARALEEVRRSATSEEDLKEALDRAREASERG |
| | QNPAESLKEAAEELKKKKEKSSD (SEQ ID NO: 449) |
| DHR36 | SDLEKALKRFVKEEKKKGRNPEEAKKEAKKLKKKLKKSAGSSDLLTA |
| | LAKFVLEEVRKGRNPEEAVKEAIKLAEKLKRSAGSSDLLTALAKFVLE |
| | EVRKGRNPEEAVKEAIKLAEKLKRSAGSSEQLEKLATKVLEEVKKGR |
| | NPKRAVEEAIKQAKEDRKRSNS (SEQ ID NO: 450) |
| DHR37 | SSTERAAQSVKKYLQQQGKDPDQAQKKAQEVKENIEKEANSSSVIRA |
| | AAAVVFYLLEQGYDPDQALKKAQEVARNIENEANSSSVIRAAAAVVF |
| | YLLEQGYDPDQALKKAQEVARNIENEANSDDVIKEAAKVVYKRLEE |
| | GQDPDKALEEARKRAQKTEKKTTS (SEQ ID NO: 451) |
| DHR38 | SSTERAAQSCKKYLQQQGKDPDQAQKKAQEVKENIEKEANSSSVIRA |
| | AAACVFYLLEQGYDCDQALKKAQEVARNIENEANSSSVIRAAAACVF |
| | YLLEQGYDCDQALKKAQEVARNIENEANSDDVIKEAAKVVYKRLEE |
| | GQDCDKALEEARKRAQKTEKKTTS (SEQ ID NO: 452) |
| DHR39 | SDLQEVADRIVEQLKREGRSPEEARKEARRLIEEIKQSAGGDSELIEVA |
| | VRIVKELEEQGRSPSEAAKEAVELIERIRRAAGGDSELIEVAVRIVKEL |
| | EEQGRSPSEAAKEAVELIERIRRAAGGDSDRIKKAVELVRELEERGRSP |
| | SEAARRAVEEIQRSVEEDGGN (SEQ ID NO: 453) |
| DHR40 | SESDEVAKRISKEAKKEGRSEEEVKELVERFREAIEKLKEQGDSEAIRV |
| | AVEIADEALREGLSPEEVVELVERFVQAIQKLQENGESEAIRVAVEIAD |
| | EALREGLSPEEVVELVERFVQAIQKLQENGEEDEIQKAVETAQEQLEE |
| | GRSPKEVVETVEEQVKEVEEKQQKGE (SEQ ID NO: 454) |
| DHR41 | SDIEKAKRIADRAIDVVRKAAEKEGGSPEKIREALQQAKRCAEKLIRL |
| | VKEAQESNSSDVREAARVALEAVRVVVRAAEEKGGSPEEVVEAVCR |
| | AVRCAEKLIRLVKRAEESNSSDVREAARVALEAVRVVVRAAEEKGGS |
| | PEEVVEAVCRAVRCAEKLIRLVKRAEESNSENVRESARRALEKVLKT |
| | VQQAEEEGKSPEEVVEQVCRSVRKAEEQIRETQERERSTS (SEQ ID |
| | NO: 455) |
| DHR42 | SDAEEVKKQAEEIANRAYKTAQKQGESDSRAKKAEKLVRKAAEKLA |
| | RLIERAQKEGDSDALEVARQALEIARRAFETAKKQGHSATEAAKAFV |
| | DVVEAAISLAELIISAKRQGDSDALEVARQALEIARRAFETAKKQGHS |
| | ATEAAKAFVDVVEAAISLAELIISAKRQGDQKALEIARKALQKAKENF |
| | EEAQKRGESATQAAKRFVDTVEKEIKKAQEQIKRERKGD (SEQ ID |
| | NO: 456) |
| DHR43 | SKEEELIEKARRVAKEAIEEAKRQGKDPSEAKKAAEKLIKAVEEAVKE |
| | AKRLKEEGNSELAELISEAIQVAVEAVEEAVRQGKDPFKAAEAAAELI |
| | RAVVEAVKEAERLKREGNSELAELISEAIQVAVEAVEEAVRQGKDPF |
| | KAAEAAAELIRAVVEAVKEAERLKREGNSELAKKINDTIREAVREVQ |
| | QAVEDGKDPFEAAREAAEKIRESVERVREEEEKKRRGN (SEQ ID NO: |
| | 457) |
| DHR44 | SNEQEKKDLKKAEEAAKSPDPELIREAIERAEESGSNKAKEIILRAAEE |
| | AAKSPDPELIRLAIEAAERSGSNKAKEIILRAAEEAAKSPDPELIRLAIE |
| | AAERSGSEKAKEIIKRAAEEAQKSPDPELQKLAKEARERLG (SEQ ID |
| | NO: 458) |
| DHR45 | SSEEEELEKDAREASESGADPEWLREIVDLARESGDSEVIELAKRALEA |
| | AKSGADPEWLLRIVRQAEESGSSEVIELAKRALEAAKSGADPEWLLRI |
| | VRQAEESGSEEVIELAKRALEEAKKGKDPKELLEEVRKREESG (SEQ |
| | ID NO: 459) |
| DHR46 | STKEEKERIERIEKEVRSPDPENIREAVRKAEELLRENPSTEAEELLRRA |
| | IEAAVRAPDPEAIREAVRAAEELLRENPSTEAEELLRRAIEAAVRAPDP |
| | EAIREAVRAAEELLRENPSEEAKELLRRAIESAKKAPDPEAQREAKRA |
| | EEELRKEDP (SEQ ID NO: 460) |
| DHR47 | STKEEKERIERIEKEVRSPDCENIREAVRKAEELLRENPSTEAEELLRRA |
| | IEAAVRCPDCEAIREAVRAAEELLRENPSTEAEELLRRAIEAAVRCPDC |
| | EAIREAVRAAEELLRENPSEEAKELLRRAIESAKKCPDPEAQREAKRA |
| | EEELRKEDP (SEQ ID NO: 461) |
| DHR48 | NSREEEEAKRIVKEAKKSGFDPEEVEKALREVIRVAEETGNSEALKEA |
| | LKIVEEAAKSGYDPAEVAKALAEVIRVAEETGNSEALKEALKIVEEAA |
| | KSGYDPAEVAKALAEVIRVAEETGNPEELKEALKRVLEAAKRGEDPA |
| | QVAKELAEEIRRNQEEG (SEQ ID NO: 462) |
| DHR49 | DSEEEQERIRRILKEARKSGTEESLRQAIEDVAQLAKKSQDSEVLEEAI |
| | RVILRIAKESGSEEALRQAIRAVAEIAKEAQDSEVLEEAIRVILRIAKES |
| | GSEEALRQAIRAVAEIAKEAQDPRVLEEAIRVIRQIAEESGSEEARRQA |
| | ERAEEEIRRRAQ (SEQ ID NO: 463) |
| DHR50 | DPEEVRREVERATEEYRKNPGSDEAREQLKEAVERAEEAARSPDPEA |
| | VQVAVEAATQIYENTPGSEEAKKALEIAVRAAENAARLPDPEAVQVA |
| | VEAATQIYENTPGSEEAKKALEIAVRAAENAARLPDPEAVRVAEEAA |
| | DQIRKNTPGSELAKRADEIKKRARELLERLP (SEQ ID NO: 464) |
| DHR51 | QSEDRKEKIRELERKARENTGSDEARQAVKEIARIAKEALEEGNADTA |
| | KEAIQRLEDLARDYSGSDVASLAVKAIAKIAETALRNGYADTAKEAIQ |
| | RLEDLARDYSGSDVASLAVKAIAKIAETALRNGYKETAEEAIKRLREL |
| | AEDYKGSEVAKLAEEAIERIEKVSRERG (SEQ ID NO: 465) |
| DHR52 | QCEDRKEKIRELERKARENTGSDEARQAVKEIARIAKEALEEGCCDTA |
| | KEAIQRLEDLARDYSGSDVASLAVKAIAKIAETALRNGCCDTAKEAIQ |
| | RLEDLARDYSGSDVASLAVKAIAKIAETALRNGCKETAEEAIKRLREL |
| | AEDYKGSEVAKLAEEAIERIEKVSRERG (SEQ ID NO: 466) |
| DHR53 | SNDEKEKLKELLKRAEELAKSPDPEDLKEAVRLAEEVVRERPGSNLA |
| | KKALEIILRAAEELAKLPDPEALKEAVKAAEKVVREQPGSNLAKKALE |
| | IILRAAEELAKLPDPEALKEAVKAAEKVVREQPGSELAKKALEIIERAA |
| | EELKKSPDPEAQKEAKKAEQKVREERPG (SEQ ID NO: 467) |
| DHR54 | TTEDERRELEKVARKAIEAAREGNTDEVREQLQRALEIARESGTTEAV |
| | KLALEVVARVAIEAARRGNTDAVREALEVALEIARESGTTEAVKLAL |
| | EVVARVAIEAARRGNTDAVREALEVALEIARESGTEEAVRLALEVVK |
| | RVSDEAKKQGNEDAVKEAEEVRKKIEEESG (SEQ ID NO: 468) |
| DHR55 | SSVAEEIEKRAKKISKELKKEGKNPEWIEELQRAADKLVEVARRATSS |
| | DALEIAKRAVKIAEELAKQGSNPKWIAELLKAAAKLVEVAARATSSD |
| | ALEIAKRAVKIAEELAKQGSNPKWIAELLKAAAKLVEVAARATSPKA |
| | LKQAKEAVKEAEELAKKGRNPKEIAEELKKRAKEVEKLARST (SEQ |
| | ID NO: 469) |
| DHR56 | SSVAEEIEKRCKKISKELKKEGKNPEWIEELQRACDKLVEVARRATSS |
| | DALEIAKRCVKIAEELAKQGSNPKWIAELLKACAKLVEVAARATSSD |
| | ALEIAKRCVKIAEELAKQGSNPKWIAELLKACAKLVEVAARATSPKA |
| | LKQAKECVKEAEELAKKGRNPKEIAEELKKCAKEVEKLARST (SEQ |
| | ID NO: 470) |
| DHR57 | STEELKKVLERVRELSERAKESTDPEEALKIAKEVIELALKAVKEDPST |
| | DALRAVLEAVRLASEVAKRVTDPDKALKIAKLVIELALEAVKEDPST |
| | DALRAVLEAVRLASEVAKRVTDPDKALKIAKLVIELALEAVKEDPSEE |
| | AKRAVEEAKRLAEEVSKRVTDPELSEKIRQLVKELEEEAQKEDP (SEQ |
| | ID NO: 471) |
| DHR58 | STEELKKVLERVRELCERAKESTDPEEALKIAKEVIELALKAVKEDPST |
| | DALRAVLEAVRCACEVAKRVTDPDKALKIAKLVIELALEAVKEDPST |
| | DALRAVLEAVRCACEVAKRVTDPDKALKIAKLVIELALEAVKEDPSE |
| | EAKRAVEEAKRCAEEVSKRVTDPELSEKIRQLVKELEEEAQKEDP |
| | (SEQ ID NO: 472) |
| DHR59 | KTEVEKKAKEVIKEAKELAKELDSEEAKKVVERIKEAAEAAKRAAEQ |
| | GKTEVAKLALKVLEEAIELAKENRSEEALKVVLEIARAALAAAQAAE |
| | EGKTEVAKLALKVLEEAIELAKENRSEEALKVVLEIARAALAAAQAA |
| | EEGKSDEARDALRRLEEAIEEAKENRSKESLEKVREEAKEAEQQAED |
| | AREG (SEQ ID NO: 473) |
| DHR60 | TDIKKKAEEIIKEAKKQGSEDAIRLAQEAKKQGTDILVRAAEIVVRAQ |
| | EQGSEDAIRLAKEASREGTDILVRAAEIVVRAQEQGSEDAIRLAKEAS |
| | REGTPTLVKAAEKVVRAQQKGSQDTIEKAKEESREG (SEQ ID NO: |
| | 474) |
| DHR61 | TDIKKKAEEIIKEAKKQGSEDAIRLAQECKKQGTDICVRAAEIVVRAQ |
| | EQGSEDAIRLAKECSREGTDICVRAAEIVVRAQEQGSEDAIRLAKECSR |
| | EGTPTCVKAAEKVVRAQQKGSQDTIEKAKEESREG (SEQ ID NO: 475) |
| DHR62 | DNDEKRKRAEKALQRAQEAEKKGDVEEAVRAAQEAVRAAKESGDN |
| | DVLRKVAEQALRIAKEAEKQGNVEVAVKAARVAVEAAKQAGDNDV |
| | LRKVAEQALRIAKEAEKQGNVEVAVKAARVAVEAAKQAGDQDVLR |
| | KVSEQAERISKEAKKQGNSEVSEEARKVADEAKKQTG (SEQ ID NO: |
| | 476) |
| DHR63 | DPDEDRERLKEELKKIREALREAKEKPDPEEIKRALREVLEAIRRILKL |
| | AERAGDPDLAREALKEINKVIREALEIAKRVPDPEVIKEALRVVLEAIR |
| | AILKLAEQAGDPDLAREALKEINKVIREALEIAKRVPDPEVIKEALRVV |
| | LEAIRAILKLAEQAGDPDLAREALEEIDK VIDEAQEISERVPDEEVQRE |
| | AQEVIKEADRARKKLSEQSG (SEQ ID NO: 477) |
| DHR64 | DPEDELKRVEKLVKEAEELLRQAKEKGSEEDLEKALRTAEEAAREAK |
| | KVLEQAEKEGDPEVALRAVELVVRVAELLLRIAKESGSEEALERALR |
| | VAEEAARLAKRVLELAEKQGDPEVALRAVELVVRVAELLLRIAKESG |
| | SEEALERALRVAEEAARLAKRVLELAEKQGDPEVARRAVELVKRVAE |
| | LLERIARESGSEEAKERAERVREEARELQERVKELREREG (SEQ ID |
| | NO: 478) |
| DHR65 | DPEDELKRVEKLVKEAEELLRQCKEKGSEECLEKALRTAEEAAREAK |
| | KVLEQAEKEGDPEVALRAVELVVRVAELLLRICKESGSEECLERALRV |
| | AEEAARLAKRVLELAEKQGDPEVALRAVELVVRVAELLLRICKESGS |
| | EECLERALRVAEEAARLAKRVLELAEKQGDPEVARRAVELVKRVAEL |
| | LERICRESGSEECKERAERVREEARELQERVKELREREG (SEQ ID NO: |
| | 479) |
| DHR66 | TSDDDKVREAEERVREAIERIQRALKKRDTPDARKALEAAKKLLKVV |
| | KLAEVVYKAAESGTSDAIKVAEAAARVAEAIARILEALNERDTPDAR |
| | KALRAAIKLAEVVYKAAESGTTEALKVAEKAARVAEKIARILEKLNE |
| | RDTPEARKKLRQAIKEAEKVYKESEQG (SEQ ID NO: 480) |
| DHR67 | TSEIDKLIKKLRQTAKEVKREAEERKRRSTDPTVREVIERLAQLALDV |
| | AEEAARLIKKATTSEVAKLVWKLARTAIEVIREAIERAERSTDPEVIRV |
| | ILELARLAAEVAKEAARLIVKATTSEVAKLVWKLARTAIEVIREAIERA |
| | ERSTDPEVIRVILELARLAAEVAKEAARLIVKATTEEVAKKVWKEAYR |
| | AIEEIRKAIEKAERSTDPNEIKKILEEARKKAEEAIERAKEIVKST (SEQ |
| | ID NO: 481) |
| DHR68 | TPRERLEEAKERVEEIRELIDKARKLQEQGNKEEAEKVLREAREQIRE |
| | VTRELEEIAKNSDTPELALRAAELLVRLIKLLIEIAKLLQEQGNKEEAE |
| | KVLREATELIKRVTELLEKIAKNSDTPELALRAAELLVRLIKLLIEIAKL |
| | LQEQGNKEEAEKVLREATELIKRVTELLEKIAKNSDTPELAKRAAELL |
| | KRLIELLKEIAKLLEEEGNEDEAEKVKEEAKELEERVRELEERIRKNSD |
| | (SEQ ID NO: 482) |
| DHR69 | NPQEDLERAEKVVRSVEEVLQRAKEAQREGDKEKVERLIKEAENQIR |
| | KARELLERVVRQNPDDPEVLLRVAELIVRLVEVVLELAKLAEKNGDK |
| | EQVERLIQTAEELIREARELLERVSREIPDNPEVLLRVAELIVRLVEVVL |
| | ELAKLAEKNGDKEQVERLIQTAEELIREARELLERVSREIPDNPESLKR |
| | VAELIKRLVKVVDELSKLAERNGDRDQVERLRQLAEELRREAEELEE |
| | RVRRERPD (SEQ ID NO: 483) |
| DHR70 | STEEKIEEARQSIKEAERSLREGNPEKAREDVRRALELVRELEKLARKT |
| | KTGSTEVLIEAARLAIEVARVALKVGSPETAREAVRTALELVQELERQ |
| | ARKTGSDEVLKRAAELAKEVARVAKEVGSPETARQARETAERLREEL |
| | RRNREKKG (SEQ ID NO: 484) |
| DHR71 | DPEEILERAKESLERAREASERGDEEEFRKAAEKALELAKRLVEQAKK |
| | EGDPELVLEAAKVALRVAELAAKNGDKEVFKKAAESALEVAKRLVE |
| | VASKEGDPELVLEAAKVALRVAELAAKNGDKEVFKKAAESALEVAK |
| | RLVEVASKEGDPELVEEAAKVAEEVRKLAKKQGDEEVYEKARETAR |
| | EVKEELKRVREEKG (SEQ ID NO: 485) |
| DHR72 | DSTKEKARQLAEEAKETAEKVGDPELIKLAEQASQEGDSEKAKAILLA |
| | AEAARVAKEVGDPELIKLALEAARRGDSEKAKAILLAAEAARVAKEV |
| | GDPELIKLALEAARRGDSEKARAILEAAERAREAKERGDPEQIKKARE |
| | LAKRG (SEQ ID NO: 486) |
| DHR73 | DAEEEAKEAIKRAQEAIELARKGNPEEARKVAEEARERAERVREEAE |
| | KRGDAEVLALVAIALALVAIALAEVGNPEEAREVAERAKEIAERVREL |
| | AEKRGDAEVLALVAIALALVAIALAEVGNPEEAREVAERAKEIAERV |
| | RELAEKRGDARVLKLVAKALELVAEALKKVGNPEEAREVEERAREIK |
| | ERVRRLLEEKG (SEQ ID NO: 487) |
| DHR74 | DSEADRIIKKLQKEIKEVEQEARDSNDDEERELLKRLAEALKRAAEAV |
| | KRAQESGDSEAIRIIKKLVKEITEVVREARKSTDKEEIELLIRLAEALAR |
| | AAEAVADAAKSGDSEAIRIIKKLVKEITEVVREARKSTDKEEIELLIRL |
| | AEALARAAEAVADAAKSGDQEAIKRIKKLVKKIIEVVRKARKSTNKK |
| | EIEKLIRKAEKLARKAEQIAEDAKRG (SEQ ID NO: 488) |
| DHR75 | DSEKEKATELAERAQDVASRVEEEARREGSRELIEIARELRERAEEAS |
| | QEGDSEKAKAILLAAKAVLVAVEVYERAKROGSDELREIARELAKEA |
| | LRAAQEGDSEKAKAILLAAKAVLVAVEVYERAKROGSDELREIAREL |
| | AKEALRAAQEGDSEKARAILEAAREVLRAVEQYERAKRRGDDDERE |
| | RAREEAREALERAREG (SEQ ID NO: 489) |
| DHR76 | NPELEEWIRRAKEVAKEVEKVAQRAEEEGNPDLRDSAKELRRAVEEA |
| | IEEAKKQGNPELVEWVARAAKVAAEVIKVAIQAEKEGNRDLFRAALE |
| | LVRAVIEAIEEAVKQGNPELVEWVARAAKVAAEVIKVAIQAEKEGNR |
| | DLFRAALELVRAVIEAIEEAVKQGNPELVERVARLAKKAAELIKRAIR |
| | AEKEGNRDERREALERVREVIERIEELVRQG (SEQ ID NO: 490) |
| DHR77 | NSDEEEAREWAERAEEAAKEALEQAKREGDEDARRVAEELEKQAEE |
| | ARRKKDSEEAEAVYWAARAVLAALEALEQAKREGDEDARRVAEELL |
| | RQAEEAARKKNSEEAEAVYWAARAVLAALEALEQAKREGDEDARR |
| | VAEELLRQAEEAARKKNPEEARAVYEAARDVLEALQRLEEAKRRGD |
| | EEERREAEERLRQAEERARKK (SEQ ID NO: 491) |
| DHR78 | NSDEEEAREWAERAEEAAKEALEQAKREGDEDARRCAEELEKQAEE |
| | ARRKKDSEEAEAVYWAARAVLAALEALEQAKREGDEDARRCAEELL |
| | RQACEAARKKNSEEAEAVYWAARAVLAALEALEQAKREGDEDARR |
| | CAEELLRQACEAARKKNPEEARAVYEAARDVLEALQRLEEAKRRGD |
| | EEERREAEERLRQACERARKK (SEQ ID NO: 492) |
| DHR79 | SSDEEEARELIERAKEAAERAQEAAERTGDPRVRELARELKRLAQEAA |
| | EEVKRDPSSSDVNEALKLIVEAIEAAVRALEAAERTGDPEVRELAREL |
| | VRLAVEAAEEVQRNPSSSDVNEALKLIVEAIEAAVRALEAAERTGDPE |
| | VRELARELVRLAVEAAEEVQRNPSSEEVNEALKKIVKAIQEAVESLRE |
| | AEESGDPEKREKARERVREAVERAEEVQRDPS (SEQ ID NO: 493) |
| DHR80 | NSEELERESEEAERRLQEARKRSEEARERGDLKELAEALIEEARAVQE |
| | LARVASERGNSEEAERASEKAQRVLEEARKVSEEAREQGDDEVLALA |
| | LIAIALAVLALAEVASSRGNSEEAERASEKAQRVLEEARKVSEEAREQ |
| | GDDEVLALALIAIALAVLALAEVASSRGNKEEAERAYEDARRVEEEA |
| | RKVKESAEEQGDSEVKRLAEEAEQLAREARRHVQETRG (SEQ ID NO: |
| | 494) |
| DHR81 | NSEELERESEEAERRLQEARKRSEEARERGDLKELAEALIEEARAVQE |
| | LARVACERGNSEEAERASEKAQRVLEEARKVSEEAREQGDDEVLALA |
| | LIAIALAVLALAEVACCRGNSEEAERASEKAQRVLEEARKVSEEAREQ |
| | GDDEVLALALIAIALAVLALAEVACCRGNKEEAERAYEDARRVEEEA |
| | RKVKESAEEQGDSEVKRLAEEAEQLAREARRHVQECRG (SEQ ID NO: |
| | 495) |
| DHR82 | NDEEVQEAVERAEELREEAEELIKKARKTGDPELLRKALEALEEAVR |
| | AVEEAIKRNPDNDEAVETAVRLARELKKVAEELQERAKKTGDPELLK |
| | LALRALEVAVRAVELAIKSNPDNDEAVETAVRLARELKKVAEELQER |
| | AKKTGDPELLKLALRALEVAVRAVELAIKSNPDNEEAVETAKRLAEE |
| | LRKVAELLEERAKETGDPELQELAKRAKEVADRARELAKKSNPN |
| | (SEQ ID NO: 496) |
| DHR83 | NDEEVQEACERAEELREEAEELIKKARKTGDPELLRKALEALEEAVRA |
| | VEEAIKRNPDNDECVETACRLARELKKVAEELQERAKKTGDPELLKL |
| | ALRALEVAVRAVELAIKSNPDNDECVETACRLARELKKVAEELQERA |
| | KKTGDPELLKLALRALEVAVRAVELAIKSNPDNEECVETAKRLAEEL |
| | RKVAELLEERAKETGDPELQELAKRAKEVADRARELAKKSNPN (SEQ |
| | ID NO: 497) |

As used throughout the present application, the term “polypeptide” is used in its broadest sense to refer to a sequence of subunit amino acids. The polypeptides of the invention may comprise L-amino acids, D-amino acids (which are resistant to L-amino acid-specific proteases in vivo), or a combination of D- and L-amino acids. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

As will be understood by those of skill in the art, the polypeptides of the invention may include additional residues at the N-terminus, C-terminus, or both that are not present in the polypeptides of Tables 1-2; these additional residues are not included in determining the percent identity of the polypeptides of the invention relative to the reference polypeptide. In one embodiment, the polypeptide comprises at least one conservative amino acid substitution. As used herein, “conservative amino acid substitution” means amino acid or nucleic acid substitutions that do not alter or substantially alter polypeptide or polynucleotide function or other characteristics. A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. As noted above, the polypeptides of the invention may include additional residues at the N-terminus, C-terminus, or both. Such residues may be any residues suitable for an intended use, including but not limited to detection tags (i.e.: fluorescent proteins, antibody epitope tags, etc.), linkers, ligands suitable for purposes of purification (His tags, etc.), and peptide domains that add functionality to the polypeptides.

In another embodiment, the invention provides protein assemblies, comprising a plurality of polypeptides of the present invention having the same amino acid sequence. As disclosed herein, the polypeptides of the invention represent novel repeat proteins with precisely specified geometries, and thus self-assemble into the protein assemblies of the invention.

In a further aspect, the present invention provides isolated nucleic acids encoding a polypeptide of the present invention. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present invention provides recombinant expression vectors comprising the isolated nucleic acid of any aspect of the invention operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting host cells is well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the host organisms either as an episome or by integration into host chromosomal DNA. In various embodiments, the expression vector may comprise a plasmid, viral-based vector, or any other suitable expression vector.

In a further aspect, the present invention provides host cells that comprise the recombinant expression vectors disclosed herein, wherein the host cells can be either prokaryotic or eukaryotic. The cells can be transiently or stably engineered to incorporate the expression vector of the invention, using standard techniques in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, N.Y.). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium. Methods to recover polypeptide from cell free extracts or culture medium are well known to the person skilled in the art.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

### Examples

In repeat proteins, the interactions between adjacent units define the shape and curvature of the overall structure⁶. While in nature the sequences of these units generally differ, highly stable repeat proteins with identical units^{7,8} have been designed for several families⁹⁻²¹ and, for leucine rich repeats, customized designed units allow control of curvature²² and new architectures¹⁷. All designed repeat structures to date have been based on naturally occurring repeat protein families. These families may cover all stable repeat protein structures that can be built from the 20 amino acids or, alternatively, natural evolution may only have sampled a subset of what is possible.

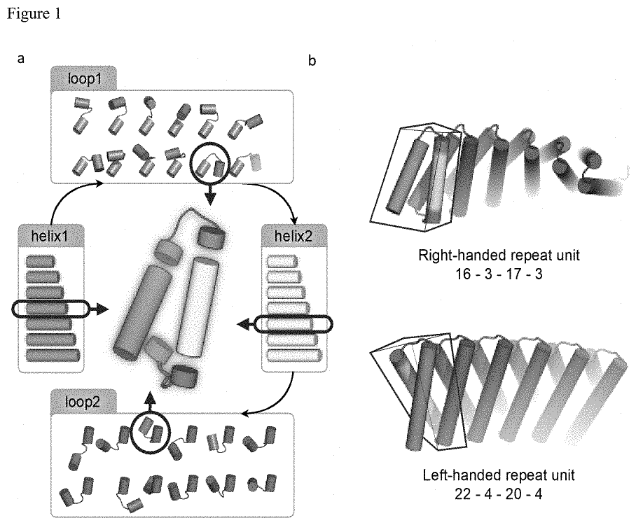

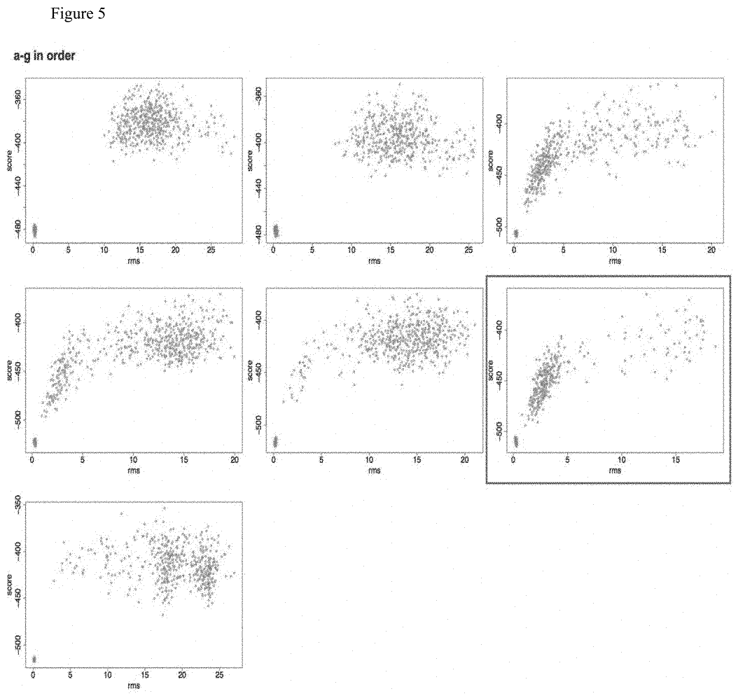

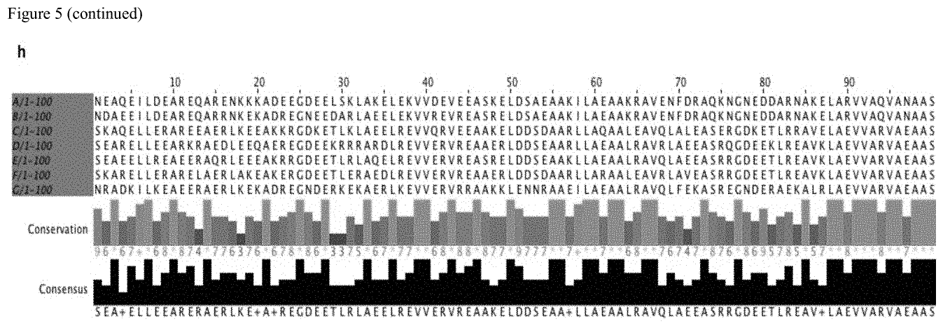

To explore the range of possible repeat protein structures, we generated new repeat protein backbone arrangements and designed sequences predicted to fold into these structures (FIG. 1). Our designs are entirely de novo; they are not based on naturally occurring repeat proteins. We focused on helix-loop-helix-loop as the basic repeating unit, as this is the simplest unit from which a wide diversity of curvatures can be generated (the simpler single helix-loop unit generates only straight rod-like models). The lengths of the two helices were varied between 10 and 28 residues, and the lengths of the two turns, from 1 to 4 residues. Starting conformations for four tandem repeats of each of the 5776 (19×19×4×4) combinations of helix and loop lengths were generated by setting the backbone torsion angles to ideal helix values for helices and extended chain values for loops. Rosetta Monte Carlo fragment assembly²³ was carried out to generate compact structures; each Monte Carlo move was made at the equivalent position in each repeat to preserve symmetry²⁰. Rosetta design calculations²⁴ were then used to identify low energy amino acid sequences with good core packing²⁵. At each step in the Monte Carlo—simulated annealing design process, a position is picked at random, and the current residue is replaced by a randomly selected amino acid and side chain conformation (rotamer); a detailed all-atom energy function is then evaluated. Identical substitutions were carried out in each copy at each move to maintain sequence identity between the four repeats; exposed hydrophobic residues in the N- and C-terminal repeats were switched to polar residues in a second round of sequence design, generating specialized capping repeats. All steps in the design process were completely automated, and the calculations were carried out without manual intervention. Designs with low energies and complementary core side chain packing were identified, and for the amino acid sequence of each of these designs, multiple independent Rosetta de novo folding trajectories²⁶ were carried out starting from an extended chain. The structures and energies of the sampled conformations map out an energy landscape for each protein (FIG. 5).

Designed helical repeat proteins (DHRs), for which the design model had much lower energy than any other conformations sampled in the de novo folding trajectories, were selected and found to span a wide array of architectures. As the rigid body transform relating adjacent repeat units is identical throughout each design by construction, and since the repeated application to an object of an identical rigid body transformation produces a helical array, the designs all have an overall helical structure⁶. It is thus convenient to classify these architectures based on three parameters defining a helix²²: the radius (r), the twist between adjacent repeats around the helical axis (w) and the translation between adjacent repeats along the helical axis (z). Because the repeat units are connected and form well packed structures, the three parameters are coupled. The arc length in the x-y plane spanned by a repeat unit is ˜r·ω, and the total length of a unit is ˜sqrt((rω)²+z²), hence the radius(r)-twist(ω) distribution has a hyperbolic shape with highly twisted structures having a smaller radius. Models with high r and high ω do not form a continuous protein core and are discarded during the backbone generation. Similarly, low energy structures do not have high (>16 Å) z values as helices in adjacent repeats cannot then closely pack. Despite these geometric constraints, the wide range of helical parameters observed in the design models highlights the high level of complexity that can be generated even for a pair of helices. In contrast, native helical repeat proteins span a much narrower range of helical parameters with very few straight (high r, low ω) or highly twisted (low r, high ω) geometries.

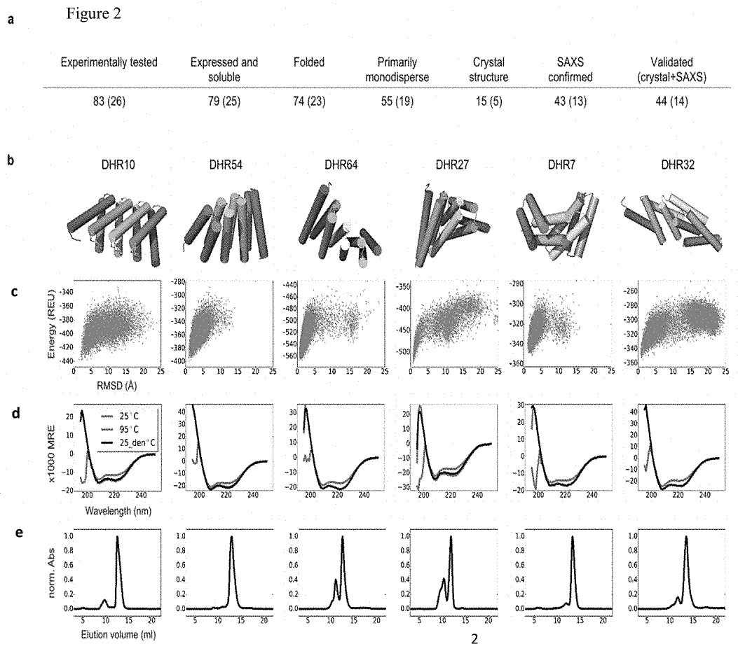

We selected for experimental characterization 83 designs spanning the range of α-helix and loop lengths and overall helical architectures; 26 of these contain disulphide bonds. For each of the designs, we obtained a synthetic gene encoding an N-terminal capping repeat, two internal repeats, and a C-terminal capping repeat including a 6-histidine tag. The proteins were expressed in Escherichia coli and purified by affinity chromatography. 74 of the 83 designs were expressed solubly and had the expected alpha helical CD spectrum at 25° C., and 72 were stably folded at 95° C. 55 of these (66% of the original experimental set) were predominantly monomeric by analytical size exclusion chromatography coupled to multi-angle light scattering (SEC-MALS); DHR49 and DHR76 were dimeric in solution. This group had the same fraction of proteins with disulphide bonds as the initial set (FIG. 2a), indicating that disulphide bonds did not provide any particular advantage in expression, solubility, or folding efficiency by further stabilizing the fold. Representative data on six of the designs are shown in FIG. 2b.

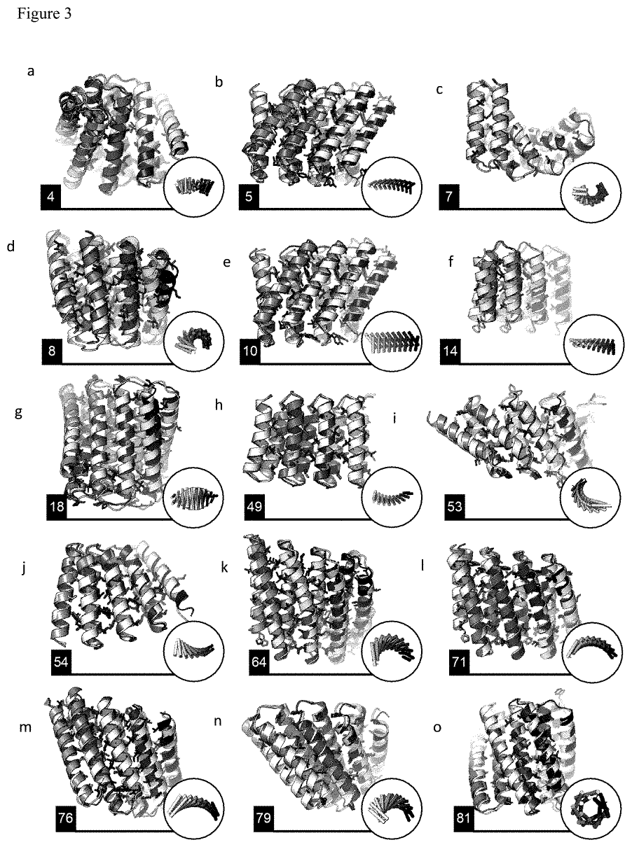

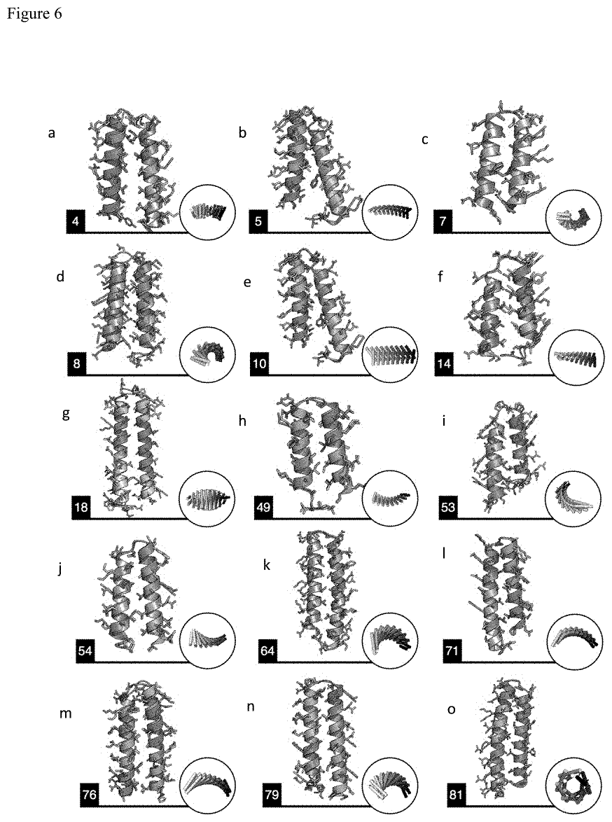

We solved the crystal structures of 15 of the designs (FIG. 3) with resolutions between 1.20 Å and 3.35 Å. The design models closely match the crystal structures with Ca RMSDs from 0.7 Å to 2.5 Å and recapitulate the side chain orientations within the hydrophobic core (FIGS. 3 and 6). The designed disulfide bonds are all formed in the structures of DHR4 and DHR7 but not in the structures of DHR5 and DHR18 due to slight structural shifts relative to the design models. The accuracy of the design models was sufficiently high that all of the crystal structures but DHR5 could be solved by molecular replacement. These repeat proteins are among the largest crystallographically validated protein structures designed completely de novo, ranging in size from 171 residues for DHR49 to 238 residues for DHR64. The crystal structures illustrate both the wide range of twist and curvature sampled by our repeat protein generation process and the accuracy with which these can be designed.

To characterize the structures for proteins that were reticent to crystallization and analyze all 55 proteins in solution, we used small angle X-ray scattering (SAXS)^{27,28}. We collected SAXS profiles for each design, and compared them to scattering profiles calculated from the design models and from crystal structures. For 43 of the designs, the radius of gyration, molecular weight, and distance distributions computed from the SAXS data corresponded to those computed from the models. For DHR49 and DHR76, we used the dimer orientation in the crystal for the fitting; the crystallographically confirmed DHR5 was unsuitable for SAXS as it formed higher order species. To further assess the fit between models and experimental data, we employed the volatility ratio (Vr), which is more robust to experimental noise than the traditional χ² comparison used in SAXS²⁹. We used the Vr values of the design models confirmed by crystallography for calibration; designs for which the Vr value between model and experimental data was less than 2.5 were considered successful. All 43 designs with radii, molecular weights, and distances consistent with the SAXS data are below the Vr threshold. Furthermore, for almost all of the designs, the theoretical scattering profile computed from the design model more closely matches its own experimental scattering profile than the experimental scattering profiles of structurally dissimilar designs.

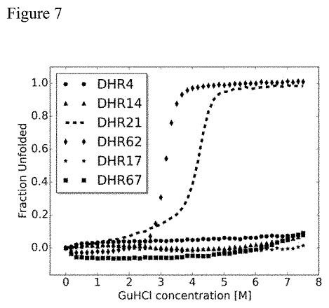

The crystallographic and SAXS data together structurally validate 44 of the 55 designs that were folded and monodisperse—more than half of the 83 that were experimentally characterized. We randomly selected two designs confirmed by crystallography, two confirmed by SAXS, and two not confirmed by SAXS, and examined their guanidine hydrochloride (GuHCl) unfolding profiles. In contrast to almost all native proteins, four of the six designs do not denature at GuHCl concentrations up to 7.5 M; the other two, which were confirmed by SAXS but did not yield crystals, have denaturation midpoints above 3 M (FIG. 7). Hence, even the apparent failures are well folded proteins; small amounts of association may be responsible for the discrepancies between computed and observed SAXS spectra rather than deviations from the design models.

We show here that a wide range of novel repeat proteins can be generated by tandem repeating a simple helix-loop-helix-loop building block. As illustrated by the comparison of design models to the corresponding crystal structures (FIG. 3), our approach allows precise control over structural details throughout a broad range of geometries and curvatures. The design models and sequences are remarkably different from each other and from naturally occurring repeat proteins, without any significant sequence or structural homology to known proteins. This work achieves key milestones in computational protein design: the design protocol is completely automatic, the folds are unlike those in nature, more than half of the experimentally tested designs have the correct overall structure as assessed by SAXS, and the crystal structures demonstrate precise control over backbone conformation for proteins over 200 amino acids. The observed level of control over the repeating helix-loop-helix-loop architecture shows that computational protein design has matured to the point of providing alternatives to naturally occurring scaffolds, including graded and tunable variation difficult to achieve starting from existing proteins. We anticipate that the 44 successful designs described in this work, and sets generated using similar protocols for other repeat units, will be widely useful starting points for the design of new protein functions and assemblies.

Naturally occurring repeat protein families, such as ankyrins, leucine rich repeats, TAL effectors and many others, play central roles in biological systems and in current molecular engineering efforts. Our results suggest that these families are only the tip of the iceberg of what is possible for polypeptide chains: there are clearly large regions of repeat protein space that are not sampled by currently known repeat protein structures. Repeat protein structures similar to our designs may not have been characterized yet, or perhaps may simply not exist in nature.

### Methods

### Similarity Search.

BLAST^{30,31} and HHSEARCH³² sequence similarity searches were performed with default settings. HHSEARCH was run on Pfam³³. Sequence alignments were depicted using Jalview³⁴. The structural similarity between designs and known helical repeat proteins was assessed by TM-align³⁵ on RepeatsDB ³⁶ representative structures.

### Protein Expression and Characterization.

Genes were synthesized and cloned in vector pET21 by GenScript (Piscataway, N.J.). Proteins were expressed in E. coli BL21(DE3), induced with 250 uM isopropyl-β-D-thiogalactopyransoide (IPTG) overnight at 22° C. and purified by metal ion affinity chromatography (IMAC) and size exclusion chromatography (SEC) as described by Parmeggiani et al.²⁰ Cells were lysed by sonication and the clarified lysate was loaded on a NiNTA superflow column (Qiagen). Lysis and washing buffer was Tris 50 mM, pH 8, NaCl 500 mM, imidazole 30 mM, glycerol 5% v/v. Lysozyme (2 mg/ml), DNAseI (0.2 mg/ml) and protease inhibitor cocktail (Roche) were added to the lysis buffer before sonication. Proteins were eluted in Tris 50 mM, pH 8, NaCl 500 mM, imidazole 250 mM, glycerol 5% v/v and dialyzed overnight either in tris 20 mM, pH 8, NaCl 150 mM. Protein concentrations were determined using a NanoDrop spectrophotometer (Thermo Scientific). Except as indicated above, enzymes and chemicals were purchased from Sigma-Aldrich. Secondary structure content, thermal stability and denaturation in presence of guanidine hydrochloride (GuHCl) were monitored by Circular Dichroism using an AVIV 420 spectrometer (Aviv Biomedical, Lakewood, N.J.). Thermal denaturation was followed at 220 nm in Tris 20 mM, 50 mM NaCl, pH 8. Proteins were considered folded if they had the expected alpha helical CD spectrum at 25° C. and had either a sharp transition in thermal denaturation or a loss of less than 20% of 220 nm CD signal at 95° C. Chemical denaturation was monitored in a 1 cm path-length cuvette at 222 nm with protein concentration of 0.05 mg/ml in phosphate buffer 25 mM NaCl 50 mM pH 7. The GuHCl concentration was automatically controlled by a Microlab titrator (Hamilton). Oligomeric state was assessed by Analytical Gel Filtration coupled to Multiple Angle Light Scattering (AFG-MALS). A Superdex 75 10/300 GL column (or superdex200 increase for DHR59, 84, 93) (GE Healthcare) equilibrated in Tris 20 mM, NaCl 150 mM, pH 8 was used on a HPLC LC 1200 Series (Agilent Technologies) connected to a miniDAWN TREOS (Wyatt Technologies). Protein molecular weights were confirmed by mass spectrometry on a LCQ Fleet Ion Trap Mass Spectrometer (Thermo Scientific). 74 of the 83 designs were expressed solubly and had the expected alpha helical CD spectrum at 25° C. 72 were stably folded at 95° C. DHR36 has Tm=75° C. and DHR13 has a broad transition with Tm=62° C. Fifty-five of these were predominantly monodisperse. DHR49 and 76 were dimeric in solution.

### Crystallization.

Proteins were purified using NiNTA resin and SEC on a superdex 75 column (GE healthcare). Pure fractions in the gel filtration buffer (20 mM Tris pH 8.0, 150 mM NaCl) were pooled and concentrated for crystallography. Initial crystallization trials were performed using the JCSG core I-IV screens at 22° C., and crystals were optimized if necessary. Drops were set up with the Mosquito HTS using 100 nL protein and 100 nL of the well solution. Crystals were cryoprotected in the reservoir solution supplemented with ethylene glycol, then flash cooled and stored in liquid nitrogen until data collection. All diffraction data were collected at the Advanced Light Source (ALS) at beamline 8.3.1 or beamline 8.2.1. Data reduction was carried out using XDS³⁷ and HKL2000 (HKL Research). Most of the structures reported here were solved by molecular replacement using Phaser. Search models were generated by ab initio folding of the designed sequences in Rosetta and a set of the lowest energy 10-100 models was selected for molecular replacement trials. DHR5 was the only structure which could not be readily solved by molecular replacement. However, due to the presence of 6 cysteine residues in the native protein, the DHR5 structure was solved by sulfur single wavelength anomalous dispersion (S-SAD) using a dataset collected at 7235 eV. Rigid body, restrained refinement with TLS and simulated annealing were carried out in Phenix³⁸. Manual adjustment of the model was carried out in Coot³⁹. The structures were validated using the Quality Control Check v2.8 developed by JCSG, which included Molprobity⁴⁰ (publicly available at the smb.slac.stanford web site).

### SAXS.

SAXS data on SEC-purified protein were collected at the SIBYLS 12.3.1 beamline at the Advanced Light Source, LBNL^{28,41,42}. Scattering measurements were performed on 20 microliter samples and loaded into a helium-purged sample chamber, 1.5 m from the Mar165 detector. Data were collected on both the original gel filtration fractions and samples concentrated ˜2×-8× from individual fractions. Fractions prior to the void volume and concentrator eluates were used for buffer subtraction. Sequential exposures (0.5, 1, 2, and 5 s) were taken at 12 keV to maximize signal to noise with visual checks for radiation-induced damage to the protein. The data used for fitting were selected for having higher signal to noise ratio and lack of radiation-induced aggregation. In case of concentration dependency, the lowest concentration was used. Models for SAXS comparison were obtained by adding the flexible C-terminal tag present in the constructs to the original designs and the crystal structures, generating 100 trajectories for each starting model by Monte Carlo fragment insertion²³. The results were clustered in Rosetta with a cluster radius of 2 Å and the cluster centers were used for comparison to the experimental data. We used FOXS^{43,44} to calculate scattering profiles from cluster centers and fit them to the experimental data. The quality of fit between models and experimental SAXS data is usually assessed by the χ value⁴⁵ which, however, suffers from over-fitting in case of noisy datasets and domination of the low region of the scattering vector (q) on the value²⁷. To avoid artificially low values that represent false positives, we instead used Volatility Ratio (Vr)₂₉ as primary metric for fit in the range of 0.015 Å⁻¹<q<0.25 Å⁻¹ r. Vr values of models with available crystal structures range from 0.7 to 2.3. Vr=2.5 was selected as upper threshold to consider a design as validated by SAXS.

Model profiles for Vr similarity maps were obtained with a standardized fit procedure by averaging the scattering profile of the cluster centers from the five largest clusters and fitting the solvent hydration layer with parameters C1=1.015 and C2=2.0 for all the models. Vr was calculated in the range 0.04 Å⁻¹<q<0.3 Å⁻¹. The order of display was derived by shape similarity of original computational models using the program damsup⁴⁶ for superposition.

### Computational Protocol

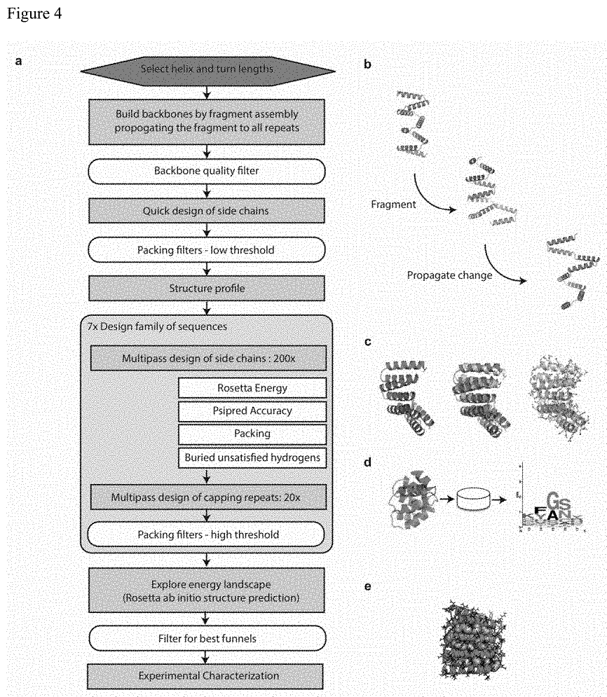

We have developed a method for construction of Designed Helical Repeats (DHRs) depicted in FIG. 4 and described below. We designed proteins based on repeating units formed by two helices and two loops. For all proteins this design process was completely automated and no manual refinement was involved. Using this protocol 69 proteins with diverse architectures were selected from the in silico candidates. For 14 models, an additional version that included disulphide bonds was selected, for a final list of 83 proteins that were experimentally tested. This design method has progressed over the duration of this research and only the final design method is described below. The database described in section 1 of the supplementary corresponds to the technique used to make DHR56-83. (a) For DHR1-4,9,11-18 the repeat backbone at the centroid level was symmetric, with first and second helices and first and second loops having the same length and conformation. The design stage was not restricted, introducing structural and sequence variability between the two halves of the repeat. (b) A higher disulfide score threshold of 1.5 was initially used which resulted in many disulfide-containing structures being non-functional. (c) We initially used ambiguous constraints between the helices. Ambiguous constraints gave a score bonus to centroid models when a helix was within 10 Å to a helix in adjacent repeat. These constraints were found to disrupt loops and result in many structures that would not fold during simulations. (d) DHR31-55 contained a displacement between helices, which resulted in highly twisted structures. This displacement was observed when the ABEGO loop types GBB and BAB were coupled with specific helix lengths. An improved sampling strategy with increased number of Monte Carlo steps was also used in these cases.

In some examples, computer software such as the Rosetta software suite (or, briefly, Rosetta), can be used to carry out at least part of the herein-described methods, protocols, and/or techniques. However, the herein-described methods and techniques are not limited to use of Rosetta or any other specific software package. For example, other software programs could be used in conjunction with this method to model multi-component symmetric protein nanostructures. As will be understood by those of skill in the art, the implementation of the design methods described herein is non-limiting, and the methods are in no way limited to the implementation disclosed herein.

Each of the following sections describes one step in Rosetta examples and corresponds to the flow chart in FIG. 4.

### 1 Backbone Design

The backbone design stage employs a simplified side chain representation (centroid)^{S1}. The backbone assembly procedure begins by picking fragments harvested directly from a non-redundant set of structures from PDB^{S2}. The fragments contain only residues that fall into the space of phi-psi backbone angles of either helices or loops depending on the desired secondary structure. Loop fragments could be further specified to fall within desired ABEGO bins³ as described by Koga et al.^{S4}.

The fragments were assembled using a Monte-Carlo sampling procedure that was initialized with ideal-helices and extended loops. After every fragment sampling step, which was allowed only in the first repeat unit and at the junction between the first and the second units, the change was propagated to all downstream repeats and scored. The score function we used considered van der Waals interactions, packing, values of backbone dihedral angles, and radius of gyration (RG) that was applied to only the first and second repeat-unit (RG-local). The RG term promotes the formation of globular proteins so applying RG to the whole model produced only highly curved structures. The sampling procedure in the database used 1500 Monte Carlo fragment insertions and was further improved to 3200 steps ordered as following: 100 Monte Carlo moves with 9 residue fragments then 100 moves with 3 residue fragments, both allowed only in loops. The loop sampling was followed by 1500 moves with 9 residue fragments and 1500 moves with 3 residue fragments, both in helices and loops (improved sampling). The improvements resulted in a 3.3 times increase of acceptance at the centroid stage. The backbone was represented as poly-tyrosine during the centroid building, maintaining enough space within the core to accommodate both small and large side chains in the design step.

Using this procedure we designed 2.88 million backbones by making 500 structures for each of 5776 different secondary structure combination.

### 2 Backbone Quality Filter: RMSD Loop Threshold and Motif Score

Designed backbones were screened for native-like features. First, loops were checked so that there was at least one 9-residue fragment from the PDB database within 0.4 Å RMSD on every position in the structure (RMSD loop threshold). To do this we used the worst9mer filter in Rosetta^{S1}. Second, the design-ability of each residue was measured by the number of pairwise side chain interactions observed in the PDB database, considering the backbone position of the two residues involved (motif score, unpublished results). Backbones with fewer than 1.5 interactions per residue were filtered out. Of the 2.88 million initial backbones 66,776 structures passed these filters.

### 3 Sequence Design—Fast

Starting from the filtered backbone conformations, we used one pass of Rosetta designs to generate repeated sequences.

### 4 Packing Filters—Low Threshold

After completing sequence design the models were filtered out if the helices were either too far apart, creating cavities in the core (poor Rosetta holes^{S5} score, >1.75), or too close together with an alanine-rich unspecific core packing (% alanine residues >25%). Of the 66,7776 structures that passed centroid 11,243 pass this filter.

### 5 Structure Profile

The structure profile biases the sequence composition towards the sequences in native proteins with similar local structure. To construct the structural profile, the sequences from the closest 100 9-residue fragments within 0.5 Å RMSD to the designed structure were used. The code to construct the structural profile is included with Rosetta as generate_struct_profile.rb in tools/fragment tools/pdb2vall. The structure profile was used in the same way as the sequence profile described by Parmeggiani et al.^{S7}

### 6 Sequence Design—Multipass

Starting from the filtered backbone conformations, we used Rosetta design to generate repeated sequences while minimizing the overall energy^{S4,S8}, increasing core packing as measured by Rosetta holes^{S6} and improving the psipred secondary structure prediction^{S9}. After the first round of sequence refinement the N and C terminal repeats (capping repeats) display exposed hydrophobic residues. The sequence design procedure was rerun for these repeats without a symmetric sequence to introduce polar amino acids.

### 7 Packing Filters—High Threshold

After completing sequence design the models were filtered out for poor packing. (holes score, <0.5). After this stage we obtained 1980 structures.

### 8 Exploration of the Energy Landscape

The designs were validated using Rosetta ab initio structure prediction using Rosetta@Home^{S10,S11}. In Rosetta ab initio prediction the energy landscape is explored using independent simulations starting from an extended structure. The distribution of the simulation results is expressed in terms of energy and distance from the target fold as root mean square deviation (RMSD). A successful design produces a distribution in the shape of a funnel with the minimum corresponding to low energy and low RMSD models and no alternative minima.

For each structure, seven family members were made from the same topology, some with increased hydrogen bond potential. Proteins where multiple family members had successful simulations were selected. The member of the family with the tightest folding funnel was chosen by visual inspection and the corresponding gene was ordered for experimental testing. Extended data FIG. 3 illustrates the folding funnel and sequence diversity for one topology.

For the database we have 761 structures that have at least one family member <3.0 RMSD from the design.

9 Add Disulphides Additional, versions with stabilizing inter-repeat disulphide bonds were also generated. Potential disulphides were scored using RosettaRemodel^{S12} and if the disulphide score was <0 they were considered.

### Time Estimates

Backbone design: on a single core of a Xeon E5-2650 took 104.5 seconds to build a structure with a 19H-2L-20H-3L topology, the median topology in the database. With an average design time of 104.5 seconds per model, it would take 3493 compute days on a single core to generate the 2.8 million structures.

Sequence design—multipass: the multipass design of sequence and capping residues takes 2.1 hours for a model with 17 length helices and 3 length loops on a single core of a Xeon E5-2650.

Exploration of the energy landscape: on a single core of a Xeon E7-2850 @ 2.00 GHZ a model with 17 residues helices and 3 residues loops is produced in 19.7 minutes. Where the computation was run on Rosetta@Home, the average was 26.7 minutes. With 7 sequences per family and a minimum of 1000 models to suitably explore the landscape it would take 130 compute days per structure.

Geometrical parameters of Designed Helical Repeat proteins
1) Global parameters
S2) Extracting parameters from naturally occurring repeats
3) Local parameters
   1) Global parameters

Class 3 repeat proteins, as described by Kajava A.^{S13}, form solenoid structures that can be described in term of global helical parameters that relate the position of one repeat to the next one: radius (r), twist or angle between adjacent repeats around the helical axis (twist, ω) and translation between adjacent repeats along the helical axis (z).

Parameters for Designed Helical Repeat proteins (DHRs) and crystal structures, together with the Cα RMSD values were measured on the two central repeats using the RepeatParameter filter available in Rosetta.

Radius and twist are inversely correlated and their distribution of whole set describes a hyperbolic shape, which can be represented as two symmetric ones, when considering the handedness of the superhelix in the co value. Handedness refers to the superhelix described by the center of mass of the repeats. z is broadly distributed, with maximum values around 16 Å.

2) Extracting Parameters from Naturally Occurring Repeats

A set of alpha-helical solenoid proteins were curated from the repeatsDB (category III.3.)^{S14} to remove both proteins that had above 90% sequence identity ^{S15,S16} and previously designed repeat proteins. After curation, 258 proteins remained out of 923. We then automatically extracted repeat units, which consisted of 3 subsequent repeats, that differed by less than 3 residues in length and had a high degree of structural similarity as measured by having a TM score ^{S17} of greater than 0.75. The requirement of high structural similarity cut down the number of repeat proteins to 81. Repeat units were identified by the method described by RAPHAEL ^{S18} implemented in Rosetta and improved. This method measures the distance from residues in the protein to random points placed around the protein. Equally spaced inflection points, where a residue was furthest or closest to these random points indicated the start of a repeat.

We found that inflection points occurred at random in repeat protein loops. To ensure each repeat was cut at the same location, the first residue in each repeat was chosen to be the loop-helix transition closest to the transition point. The code for this is available as extractNativeRepeats in Rosetta after git branch c876538. After locating repeats we assigned the class name of each repeat based on the PDB assignment in the Pfam databases^{S19}. The Rise/Omega/Twist parameters were calculated by superimposing the first repeat-unit onto the second using TM-align^{S17} then calling the parameter calculators and averaging the values within the same protein. This approach does not provide an extensive coverage of all the possible curvatures for each family but an indication of the protein average values.

### 3) Local Parameters

Local parameters describe the helix-helix interactions and, due to the repeating structures, only two interactions are needed to capture the local geometry: helix1.1-helix1.2 within a repeat and helix1.1-helix2.1 between first and second repeat. Angle between helices and distance between helix centers of mass were used as parameters, extracted with a modified version of the publicly available script that can be found at the web site pymolwiki. Secondary structure definition were assigned using DSSP^{S20}. For the two central repeats, all atoms RMSDs between crystal structures and design are reported. Repeat handedness, as defined by Kobe and Kajava^{S21}, indicates the rotation of the main chain going from the N- to the C-terminal around the axis connecting the repeat centers of mass.

### Structure and Sequence Comparison

Structural comparison of experimentally validated designs with representative repeat proteins from repeatDB^{S14} revealed that DHRs cluster in different families than the existing repeat proteins. Additionally, designs are equally distributed between right-handed and left-handed architecture, as referred to the repeat handedness (see local parameters above), in contrast to known alpha helical repeat proteins, which are mostly right-handed. This result indicates that the handedness observed is not an intrinsic limitation of repeat proteins structures but the result of a bias during evolution.

### Structure Determination Remarks

Due to the presence of 6 cysteine residues in the native protein, the DHR5 structure was solved by sulfur single wavelength anomalous dispersion (S-SAD) using a dataset collected at 7235 eV. A search for 6 individual sulfur atoms in SHELXD gave many clear solutions that led to near complete autobuilding of a poly-alanine backbone in SHELXE, which was further elaborated using the Autobuild module of Phenix. Ultimately, the final model for DHR5 was in good agreement with the design target structure, despite our initial difficulties in phasing by molecular replacement. While the SAD data set was limited to 1.85 Å, the final model was refined against the original data set (1.25 Å). Both data sets were deposited in the Protein Data Bank.

The asymmetric unit for DHR8 was found to contain 4 copies of DHR8. Although the overall structure of the 4 copies is similar, the electron density for the N-terminal helix from two of these monomers is weak, suggesting that these helices are partially disordered in the crystal. Indeed, crystal packing of these helices in the designed conformation would have led to significant steric overlap with one another. As the corresponding helices in the remaining two DHR8 monomers were well-ordered and essentially as designed, these fully ordered models were used for further analysis.

The dataset collected for DHR14 had a large non-origin Patterson peak at fractional coordinates (0.000, 0.217, 0.000), suggesting the presence of translational NCS. However, consideration of the apparent space group, unit cell parameters, and plausible solvent content strongly indicated the presence of a single copy of DHR14 in the asymmetric unit. Given the relatively low pitch of this helical design and the translational pseudosymmetry between the N- and C-terminal halves of the protein, we suspected that intramolecular pseudotranslational NCS might account for the observed Patterson peak. Ultimately, a molecular replacement solution was obtained using 4 of the 8 designed helices of DHR14, and this was sufficient to bootstrap autobuilding of the remaining backbone using SHELXE. In the final model, the helical axis of DHR14 is closely aligned with the crystallographic b axis, and pseudotranslational NCS between the N- and C-terminal repeats with a translation of ˜21 Å is in good agreement with the observed fractional Patterson peak at ˜0.22 along b.

### Small Angle X-Ray Scattering (SAXS) Analysis

Guinier and P(r) analysis were done using ATSAS²⁶. The Porod exponent was determined from a linear regression analysis (I vs q) of the top of the first peak in the Porod-Debye plot (q⁴*I(q) vs q⁴) of the scattering data, implemented in SCÅTTER, available at beamline 12.3.1^{S27,S28}. The molecular mass in solution was calculated using SCÅTTER^{S29}.

25% of the designs had molecular weights in solution that were significantly greater than the predicted molecular weight (1.2-4 fold), suggesting that these designs formed multimeric assemblies or a small portion of aggregates^{S29}. All 55 designs had Porod exponents (P_{E}) greater than 2.9, indicating significant levels of folded protein; 67% of the designs had a P_{E} of 3.4-4, indicating a well-folded core^{S28}. Of the 15 proteins that crystallized, the majority (66%) had P_{E} of 3.9-4, consistent with more well-packed proteins being easier to crystallize.

Radius of gyration (Rg) and maximum of distance distribution (dmax) were calculated from real space distance distribution P(r). Among the models confirmed by crystallography, DHR 49 and 76 formed dimers in solution. The experimental data were fit using models based on the dimer configuration observed in the crystal structure. DHR 5 tendency to aggregation (see SEC in supporting_experimental_data.pdf) affected the SAXS profile resulting in a high Molecular weight and Vr above our acceptance threshold.

If molecular mass and Rg of models were within a 25% error from experimental data and Vr was below 2.5, the models were considered able to recapture the SAXS data. Dmax errors are generally within 25%.

43 designs satisfied our requirements: DHR 1 2 3 4 7 8 9 10 14 15 18 20 21 23 24 26 27 31 32 36 39 46 47 49 52 53 54 55 57 58 59 62 64 68 70 71 72 76 77 78 79 80 81 82.

TABLE 3
Protein Sequences (including optional His-tags at C-terminus)

| name | sequence |
|---|---|
| DHR | MGCDQVAKDASSTIREVIEKNPNYSEKVADVAAKIVKKIIEGNPNGCDCVAKAASSIIRAVIEKNPNYSEV |
| 1 | VADVAAAIVKAIIEGNPNGCDCVAKAASSIIRAVIEKNPNYSEVVADVAAAIVKAIIEGNPNGRDCVRKAA |
| | SSIIRAVQEKNPNYSEVVEDVKRAIEKAIKEGNPNGWLEHHHHHH (SEQ ID NO: 498) |
| DHR | MSDADEAAKEANKAENKARNRNDDEAAKAVKLIKEAIERAKKRNESDAVEAAKEAAKALNKALNRNDDEAA |
| 2 | KAVALIAEAIIRALKRNESDAVEAAKEAAKALNKALNRNDDEAAKAVALIAEAIIRALKRNESDAVEKAKE |
| | AAKNLNKALNRNDDEQAKHVAKQAENIIRALKRNESWLEHHHHH (SEQ ID NO: 499) |
| DHR | MSSEDTVRKIAQKCSEAIRESNDCEEAARKCAKTISEAIRESNSSELAVRIIAQVCSEAIRESNDCECAAR |
| 3 | ICAKIISEAIRESNSSELAVRIIAQVCSEAIRESNDCECAARICAKIISEAIRESNSSELAKRIIKQVCSE |
| | AKRESNDTECAKRICTKIKSEAKRESNSWLEHHHHHH (SEQ ID NO: 500) |
| DHR | MSYEDECEEKARRVAEKVERLKRSGTSEDEIAEEVAREISEVIRTLKESGSSYEVICECVARIVAEIVEAL |
| 4 | KRSGTSEDEIAEIVARVISEVIRTLKESGSSYEVICECVARIVAEIVEALKRSGTSEDEIAEIVARVISEV |
| | IRTLKESGSSYEVIKECVQRIVEEIVEALKRSGTSEDEINEIVRRVKSEVERTLKESGSSWLEHHHHHH |
| | (SEQ ID NO: 501) |
| DHR | MSSEKEELRERLVKICVENAKRKGDDTEEAREAAREAFELVREAAERAGIDSSEVLELAIRLIKECVENAQ |
| 5 | REGYDISEACRAAAEAFKRVAEAAKRAGITSSEVLELAIRLIKECVENAQREGYDISEACRAAAEAFKRVA |
| | EAAKRAGITSSETLKRAIEEIRKRVEEAQREGNDISEACROAAEEFRKKAEELKRRGDGWLEHHHHHH |
| | (SEQ ID NO: 502) |
| DHR | MSEEKEEALKKVREAAKKLGSSDEEARKCFEEAREWAERTGSSAYEAAEALFKVLEAAYKLGSSAEEACEC |
| 6 | FNQAAEWAERTGSGAYEAAEALFKVLEAAYKLGSSAEEACECFNQAAEWAERTGSGAYEAAERLFEELERA |
| | YEEGSSAEEACEEFNKKEEEAHRKGKKWLEHHHHHH (SEQ ID NO: 503) |
| DHR | MSTKEDARSTCEKAARKAAESNDEEVAKQAAKDCLEVAKQAGMPTKEAARSFCEAAARAAAESNDEEVAKI |
| 7 | AAKACLEVAKQAGMPTKEAARSFCEAAARAAAESNDEEVAKIAAKACLEVAKQAGMPTKEAARSFCEAAKR |
| | AAKESNDEEVEKIAKKACKEVAKQAGMPWLEHHHHHH (SEQ ID NO: 504) |
| DHR | MSDEMKKVMEALKKAVELAKKNNDDEVAREIERAAKEIVEALRENNSDEMAKVMLALAKAVLLAAKNNDDE |
| 8 | VAREIARAAAEIVEALRENNSDEMAKVMLALAKAVLLAAKNNDDEVAREIARAAAEIVEALRENNSDEMAK |
| | KMLELAKRVLDAAKNNDDETAREIARQAAEEVEADRENNSWLEHHHHHH (SEQ ID NO: 505) |
| DHR | MSYEDEAEEKARRVAEKVERLKRSGTSEDEIAEEVAREISEVIRTLKESGSSYEVIAEIVARIVAEIVEAL |
| 9 | KRSGTSEDEIAEIVARVISEVIRTLKESGSSYEVIAEIVARIVAEIVEALKRSGTSEDEIAEIVARVISEV |
| | IRTLKESGSSYEVIKEIVQRIVEEIVEALKRSGTSEDEINEIVRRVKSEVERTLKESGSSWLEHHHHHH |
| | (SEQ ID NO: 506) |
| DHR | MSSEKEELRERLVKIVVENAKRKGDDTEEAREAAREAFELVREAAERAGIDSSEVLELAIRLIKEVVENAQ |
| 10 | REGYDISEAARAAAEAFKRVAEAAKRAGITSSEVLELAIRLIKEVVENAQREGYDISEAARAAAEAFKRVA |
| | EAAKRAGITSSETLKRAIEEIRKRVEEAQREGNDISEAARQAAEEFRKKAEELKRRGDGWLEHHHHHH |
| | (SEQ ID NO: 507) |
| DHR | MSDADEAAKEANKAENKARNRNDDEAAKAVKLCKEAIERAKKRNESDAVEAAKEAAKALNKALNRNDDEAA |
| 11 | KAVALCCEAIIRALKRNESDAVEAAKEAAKALNKALNRNDDEAAKAVALCCEAIIRALKRNESDAVEKAKE |
| | AAKNLNKALNRNDDEQAKHVAKQCENIIRALKRNESWLEHHHHHH (SEQ ID NO: 508) |
| DHR | MDDEEQCREIAEKAKQTYTDDEEIARIIAEAARQTTTDDEEICRCIAEAAKQTYTDDEEIARIIAYAARQT |
| 12 | TTDDEEICRCIAEAAKQTYTDDEEIARIIAYAARQTTTDDEEIERCIEEAAKQTYTDDEEIERIKEYARRQ |
| | TTTDGWLEHHHHHH (SEQ ID NO: 509) |
| DHR | MNAEDKAREVLKELKDEGSPEEEAARQVLKDLNREGSNAEDAARAVLKALKDEGSPEEEAARAVLKALNRE |
| 13 | GSNAEDAARAVLKALKDEGSPEEEAARAVLKALNREGSNEEDASRAVLKALKDEGSPEEEARRAVEKALNR |
| | EGSNGWLEHHHHHH (SEQ ID NO: 510) |
| DHR | MDSEEVNERVKOLAEKAKEATDKEEVIEIVKELAELAKQSTDSELVNEIVKQLAEVAKEATDKELVIYIVK |
| 14 | ILAELAKQSTDSELVNEIVKQLAEVAKEATDKELVIYIVKILAELAKQSTDSELVNEIVKQLEEVAKEATD |
| | KELVEHIEKILEELKKQSTDGWLEHHHHHH (SEQ ID NO: 511) |
| DHR | MNDERQKQREEVRKLAEELASKATDEELIKEIKKCAQLAEELASRSTNDELIKQILEVAKLAFELASKATD |
| 15 | EELIKEILKCCQLAFELASRSTNDELIKQILEVAKLAFELASKATDEELIKEILKCCQLAFELASRSTNDE |
| | EIKQILETAKEAFERASKATDEEEIKEILKKCQEKFEKKSRSTNGWLEHHHHHH (SEQ ID NO: 512) |
| DHR | MNDKAKEAEELLRKALEKAEKENDETAIRCVELLKEALERAKKNNNDKAIEAVELLAKALEKALKENDETA |
| 16 | IRCVCLLAEALLRALKNNNDKAIEAVELLAKALEKALKENDETAIRCVCLLAEALLRALKNNNDKAIEEVE |
| | RLAKELEKALKENDETKIREVCERAEELLRRLKNNNGWLEHHHHHH (SEQ ID NO: 513) |
| DHR | MSSEDAREKIEQLCREAKEIAERAKQQNSQEEAREAIEKLLRIAKRIAELAKQANQSEVAREAIECLCRIA |
| 17 | KLIAELAKOANSQEVAREAIEALLRIAKLIAELAKQANQSEVAREAIECLCRIAKLIAELAKQANSQEVAR |
| | EAIEALLRIAKLIAELAKqANQSEVAREAIECLSRIAKLIEELAKQANSQEVKREAQEALDRIQKLIEELQ |
| | KqANQGWLEHHHHHH (SEQ ID NO: 514) |
| DHR | MDIEKLCKKAESEAREARSKAEELRQRHPDSQAARDAQKLASQAEEAVKLACELAQEHPNADIAKLCIKAA |
| 18 | SEAAEAASKAAELAQRHPDSQAARDAIKLASQAAEAVKLACELAQEHPNADIAKLCIKAASEAAEAASKAA |
| | ELAQRHPDSQAARDAIKLASQAAEAVKLACELAQEHPNADIAKKCIKAASEAAEEASKAAEEAQRHPDSQK |
| | ARDEIKEASQKAEEVKERCERAQEHPNAWLEHHHHHH (SEQ ID NO: 515) |
| DHR | MDEIEKVREEAEKLKKKTDDEDVLEVAREAIRAAKEATSDEILKVIKEALKLAKKTTDKDVLEVAREAIRA |
| 19 | AEEATDDEILKVIKEALKLAKKTTDKDVLEVAREAIRAAEEATDEEILKEIKEALKKAKETTDTEELEKAR |
| | EQIRKAEESTDGWLEHHHHHH (SEQ ID NO: 516) |
| DHR | MSDIEEIRQLAEELRKKSDNEEVRKLAQEAAELAKRSTDSDVLEIVKDALELAKQSTNEEVIKLALKAAVL |
| 20 | AAKSTDSDVLEIVKDALELAKOSTNEEVIKLALKAAVLAAKSTDEEVLEEVKEALRRAKESTDEEEIKEEL |
| | RKAVEEAESTDGWLEHHHHHH (SEQ ID NO: 517) |
| DHR | MSEKEKVEELAQRIREQLPDTELAREAQELADEARKSDDSEALKVVYLALRIVQQLPDTELAREALELAKE |
| 21 | AVKSTDSEALKVVYLALRIVQQLPDTELAREALELAKEAVKSTDQEALKSVYEALQRVQDKPNTEEARESL |
| | ERAKEDVKSTDGWLEHHHHHH (SEQ ID NO: 518) |
| DHR | MDDAEELRERARDLLRKNGSSEEEIKKVDEELEKIVRKADSDDAVKLAVKAAALLAENGSSAEEIVKVLEE |
| 22 | LLKIVEKADSDDAVKLAVKAAALLAENGSSAEEIVKVLEELLKIVEKADSEEEVKDAVREAAELAERGSSA |
| | EEIRKQLKDRLRKVEESDSGWLEHHHHHH (SEQ ID NO: 519) |
| DHR | MSDSEKLAKRVLKELKRRGTSDEELERMKRELEKIIKSATSSDAMRLALRVVLELVRRGTSSEILEKMMRM |
| 23 | LIKIIQSATSSDAMRLALRVVLELVRRGTSSEILEKMMRMLIKIIQSATSDDQMREALRQVLEEVRKGTSS |
| | EQLERSMRKLIKEIKKRTSGWLEHHHHHH (SEQ ID NO: 520) |
| DHR | MSEAEELARRAAKEAKELCKRSTDEELCKELKKLAELLKELAERYPDSEAAKLALKAALEAIELCKQSTDE |
| 24 | ELCEELVKLAQKLIELAKRYPDSEAAKLALKAALEAIELCKQSTDEELCEELVKLAQKLIELAKRYPDSEE |
| | AKRALKEAKELIEQCKESTDEDECRELVKRAEELIREAKENPDGWLEHHHHHH (SEQ ID NO: 521) |
| DHR | MDERDKVRELIDRVEKELKREGTSEELIEEIRKVLKKAKEAADSDDDEAIKVAKEIVRVILELVREGTSSE |
| 25 | LIEEILKVLSLAAEAAKSTDDEAIKVAKEIVRVILELVREGTSSELIEEILKVLSLAAEAAKSTDEEAIKK |
| | AKEIVRRILELTREGTSEEEIREELKELRKKAQKAKSPEGWLEHHHHHH (SEQ ID NO: 522) |
| DHR | MDECERLRQEVEKAEKELEKLAKQSTDEEVRQIAREVAKOLRRLAEEACRSNSDECLRLASEVVKAVQELV |
| 26 | KLAEQATDEEVIRVALEVARELIRLAQEACRSNDDECLRLASEVVKAVQELVKLAEQATDEEVIRVALEVA |
| | RELIRLAQEACRSNDEECLREASEVVKEVQELVKEAEKSTDEEEIRELLQRAEERIREAQERCREGDGWLE |
| | HHHHHH (SEQ ID NO: 523) |
| DHR | MTRQKEQLDEVLEEIQRLAEEARKLMTDEEEAKKIQEEAERAKEMLRRAVEKVTDNEVIEKLLEVVKEIIR |
| 27 | LAEEAMKKMTDEEEAAKIAKEALEAIKMLARAVEEVTDNEVIEKLLEVVKEIIRLAEEAMKKMTDEEEAAK |
| | IAKEALEAIKMLARAVEEVTDKERIEQLLREVKEEIRRAEEESRKETDDEEAAKRAREALRRIRERAREVE |
| | EDKSGWLEHHHHHH (SEQ ID NO: 524) |
| DHR | MDEEVQRIREEVRRAIEEVRESLERNDSEEAEELAREALERVAEEVKESIKERPDRDLAIEAIRALVRLAI |
| 28 | EIVRLALEQNDSELAREVAEEALRAVAEVVKEAIRQRGDRDLAIEAIRALVRLAIEIVRLALEQNDSELAR |
| | EVAEEALRAVAEVVKEAIRQRGDRELAKEAIRALRRLAEEIRRLAEEQNDDELAREVEELAREAIEEVRKE |
| | LERQRPGRGWLEHHHHHH (SEQ ID NO: 525) |
| DHR | MSEVEESAQEVEKRAQEVREEAERRGTSQEVLDEIKRVVDEARQLAQRAKESDDSEVAESALQVVREALKV |
| 29 | VLSALERGTSEEVLKEILRVVSEAIKLALEAIKSSDSEVAESALQVVREALKVVLSALERGTSEEVLKEIL |
| | RVVSEAIKLALEAIKSSDSETARRALEKVRESLKEVLEQLERGTSEEELRESLREVSENIRKALEEIKSPD |
| | GWLEHHHHHH (SEQ ID NO: 526) |
| DHR | MSTVKELLDRARELMRELAERASEQGSDEEEARKLLEDLEQLVQEIRRELEETGTSSEVIRLIAKAIMLMA |
| 30 | ELALRAAEQGSDAEEAMKLLKDLLRLVLEILRELRETGTDSEVIRLIAKAIMLMAELALRAAEQGSDAEEA |
| | MKLLKDLLRLVLEILRELRETGTDKEEIRKVAEEIMRRAKTALDEARQGSDAEEAMKRLKEQLRRILERLR |
| | EEREKGTDGWLEHHHHHH (SEQ ID NO: 527) |
| DHR | MDSYTERARKAVKRYVKEEGGSEEEAEREAEKVREEIRKKASDSYLIQAAAAVVAYVIEEGGSPEEAVKIA |
| 31 | EEVVRRIKEKADDSYLIQAAAAVVAYVIEEGGSPEEAVKIAEEVVRRIKEKADDRELIRRAAERVAEVIER |
| | GGSPEEAVKEAEKEVKKQKEESDGWLEHHHHHH (SEQ ID NO: 528) |
| DHR | MSIQEKAKQSVIRKVKEEGGSEEEARERAKEVEERLKKEADDSTLVRAAAAVVLYVLEKGGSTEEAVQRAR |
| 32 | EVIERLKKEASDSTLVRAAAAVVLYVLEKGGSTEEAVORAREVIERLKKEASDEELIREAAKEVLKVLEEG |
| | GSVEEAVERARERIEELQKRSDDGWLEHHHHHH (SEQ ID NO: 529) |
| DHR | MSETEEVKKLVEEKVKKEGGSPEEAKETAKEVTEELKEESQDSTLLKVAALVASAVLKEGGSPEEAAETAK |
| 33 | EVVKELRKSASDSTLLKVAALVASAVLKEGGSPEEAAETAKEVVKELRKSASDEELLKEAARQAEESLRQG |
| | KSPEEAAEEAKKEVKKLKEKSQDGWLEHHHHHH (SEQ ID NO: 530) |
| DHR | MSETEEVKKLCEEKVKKEGGSPEEAKETAKEVTEELKEESQDSTLLKVAALCASAVLKEGGSCEEAAETAK |
| 34 | EVVKELRKSASDSTLLKVAALCASAVLKEGGSCEEAAETAKEVVKELRKSASDEELLKEAARQAEESLRQG |
| | KSCEEAAEEAKKEVKKLKEKSQDGWLEHHHHHH (SEQ ID NO: 531) |
| DHR | MSEEDEVAKQASRYAKEQGGDPEKSREEAEKALEEVKKQATSSEALQVALEAARYASEEGEDPAEALKEAA |
| 35 | RALEEVRRSATSSEALQVALEAARYASEEGEDPAEALKEAARALEEVRRSATSEEDLKEALDRAREASERG |
| | QNPAESLKEAAEELKKKKEKSSDGWLEHHHHHH (SEQ ID NO: 532) |
| DHR | MSDLEKALKRFVKEEKKKGRNPEEAKKEAKKLKKKLKKSAGSSDLLTALAKFVLEEVRKGRNPEEAVKEAI |
| 36 | KLAEKLKRSAGSSDLLTALAKFVLEEVRKGRNPEEAVKEAIKLAEKLKRSAGSSEQLEKLATKVLEEVKKG |
| | RNPKRAVEEAIKQAKEDRKRSNSGWLEHHHHHH (SEQ ID NO: 533) |
| DHR | MSSTERAAQSVKKYLQQQGKDPDQAQKKAQEVKENIEKEANSSSVIRAAAAVVFYLLEQGYDPDQALKKAQ |
| 37 | EVARNIENEANSSSVIRAAAAVVFYLLEQGYDPDQALKKAQEVARNIENEANSDDVIKEAAKVVYKRLEEG |
| | QDPDKALEEARKRAQKTEKKTTSGWLEHHHHHH (SEQ ID NO: 534) |
| DHR | MSSTERAAQSCKKYLQQQGKDPDQAQKKAQEVKENIEKEANSSSVIRAAAACVFYLLEQGYDCDQALKKAQ |
| 38 | EVARNIENEANSSSVIRAAAACVFYLLEQGYDCDQALKKAQEVARNIENEANSDDVIKEAAKVVYKRLEEG |
| | QDCDKALEEARKRAQKTEKKTTSGWLEHHHHHH (SEQ ID NO: 535) |
| DHR | MSDLQEVADRIVEQLKREGRSPEEARKEARRLIEEIKOSAGGDSELIEVAVRIVKELEEQGRSPSEAAKEA |
| 39 | VELIERIRRAAGGDSELIEVAVRIVKELEEQGRSPSEAAKEAVELIERIRRAAGGDSDRIKKAVELVRELE |
| | ERGRSPSEAARRAVEEIQRSVEEDGGNGWLEHHHHHH (SEQ ID NO: 536) |
| DHR | MSESDEVAKRISKEAKKEGRSEEEVKELVERFREAIEKLKEQGDSEAIRVAVEIADEALREGLSPEEVVEL |
| 40 | VERFVQAIQKLQENGESEAIRVAVEIADEALREGLSPEEVVELVERFVQAIQKLQENGEEDEIQKAVETAQ |
| | EQLEEGRSPKEVVETVEEQVKEVEEKQQKGEGWLEHHHHHH (SEQ ID NO: 537) |
| DHR | MSDIEKAKRIADRAIDVVRKAAEKEGGSPEKIREALQQAKRCAEKLIRLVKEAQESNSSDVREAARVALEA |
| 41 | VRVVVRAAEEKGGSPEEVVEAVCRAVRCAEKLIRLVKRAEESNSSDVREAARVALEAVRVVVRAAEEKGGS |
| | PEEVVEAVCRAVRCAEKLIRLVKRAEESNSENVRESARRALEKVLKTVQQAEEEGKSPEEVVEQVCRSVRK |
| | AEEQIRETQERERSTSGWLEHHHHHH (SEQ ID NO: 538) |
| DHR | MSDAEEVKKQAEEIANRAYKTAQKQGESDSRAKKAEKLVRKAAEKLARLIERAQKEGDSDALEVARQALEI |
| 42 | ARRAFETAKKQGHSATEAAKAFVDVVEAAISLAELIISAKROGDSDALEVARQALEIARRAFETAKKQGHS |
| | ATEAAKAFVDVVEAAISLAELIISAKROGDQKALEIARKALQKAKENFEEAQKRGESATQAAKREVDTVEK |
| | EIKKAQEQIKRERKGDGWLEHHHHHH (SEQ ID NO: 539) |
| DHR | MSKEEELIEKARRVAKEAIEEAKROGKDPSEAKKAAEKLIKAVEEAVKEAKRLKEEGNSELAELISEAIQV |
| 43 | AVEAVEEAVROGKDPFKAAEAAAELIRAVVEAVKEAERLKREGNSELAELISEAIQVAVEAVEEAVRQGKD |
| | PFKAAEAAAELIRAVVEAVKEAERLKREGNSELAKKINDTIREAVREVQQAVEDGKDPFEAAREAAEKIRE |
| | SVERVREEEEKKRRGNGWLEHHHHHH (SEQ ID NO: 540) |
| DHR | MSNEQEKKDLKKAEEAAKSPDPELIREAIERAEESGSNKAKEIILRAAEEAAKSPDPELIRLAIEAAERSG |
| 44 | SNKAKEIILRAAEEAAKSPDPELIRLAIEAAERSGSEKAKEIIKRAAEEAQKSPDPELQKLAKEARERLGG |
| | WLEHHHHHH (SEQ ID NO: 541) |
| DHR | MSSEEEELEKDAREASESGADPEWLREIVDLARESGDSEVIELAKRALEAAKSGADPEWLLRIVRQAEESG |
| 45 | SSEVIELAKRALEAAKSGADPEWLLRIVRQAEESGSEEVIELAKRALEEAKKGKDPKELLEEVRKREESGG |
| | WLEHHHHHH (SEQ ID NO: 542) |
| DHR | MSTKEEKERIERIEKEVRSPDPENIREAVRKAEELLRENPSTEAEELLRRAIEAAVRAPDPEAIREAVRAA |
| 46 | EELLRENPSTEAEELLRRAIEAAVRAPDPEAIREAVRAAEELLRENPSEEAKELLRRAIESAKKAPDPEAQ |
| | REAKRAEEELRKEDPGWLEHHHHHH (SEQ ID NO: 543) |
| DHR | MSTKEEKERIERIEKEVRSPDCENIREAVRKAEELLRENPSTEAEELLRRAIEAAVRCPDCEAIREAVRAA |
| 47 | EELLRENPSTEAEELLRRAIEAAVRCPDCEAIREAVRAAEELLRENPSEEAKELLRRAIESAKKCPDPEAQ |
| | REAKRAEEELRKEDPGWLEHHHHHH (SEQ ID NO: 544) |
| DHR | MNSREEEEAKRIVKEAKKSGFDPEEVEKALREVIRVAEETGNSEALKEALKIVEEAAKSGYDPAEVAKALA |
| 48 | EVIRVAEETGNSEALKEALKIVEEAAKSGYDPAEVAKALAEVIRVAEETGNPEELKEALKRVLEAAKRGED |
| | PAQVAKELAEEIRRNOEEGGWLEHHHHHH (SEQ ID NO: 545) |
| DHR | MDSEEEQERIRRILKEARKSGTEESLRQAIEDVAQLAKKSQDSEVLEEAIRVILRIAKESGSEEALRQAIR |
| 49 | AVAEIAKEAQDSEVLEEAIRVILRIAKESGSEEALRQAIRAVAEIAKEAQDPRVLEEAIRVIRQIAEESGS |
| | EEARROAERAEEEIRRRAQGWLEHHHHHH (SEQ ID NO: 546) |
| DHR | MDPEEVRREVERATEEYRKNPGSDEAREQLKEAVERAEEAARSPDPEAVQVAVEAATQIYENTPGSEEAKK |
| 50 | ALEIAVRAAENAARLPDPEAVQVAVEAATQIYENTPGSEEAKKALEIAVRAAENAARLPDPEAVRVAEEAA |
| | DQIRKNTPGSELAKRADEIKKRARELLERLPGWLEHHHHHH (SEQ ID NO: 547) |
| DHR | MQSEDRKEKIRELERKARENTGSDEARQAVKEIARIAKEALEEGNADTAKEAIQRLEDLARDYSGSDVASL |
| 51 | AVKAIAKIAETALRNGYADTAKEAIQRLEDLARDYSGSDVASLAVKAIAKIAETALRNGYKETAEEAIKRL |
| | RELAEDYKGSEVAKLAEEAIERIEKVSRERGGWLEHHHHHH (SEQ ID NO: 548) |
| DHR | MQCEDRKEKIRELERKARENTGSDEARQAVKEIARIAKEALEEGCCDTAKEAIQRLEDLARDYSGSDVASL |
| 52 | AVKAIAKIAETALRNGCCDTAKEAIQRLEDLARDYSGSDVASLAVKAIAKIAETALRNGCKETAEEAIKRL |
| | RELAEDYKGSEVAKLAEEAIERIEKVSRERGGWLEHHHHHH (SEQ ID NO: 549) |
| DHR | MSNDEKEKLKELLKRAEELAKSPDPEDLKEAVRLAEEVVRERPGSNLAKKALEIILRAAEELAKLPDPEAL |
| 53 | KEAVKAAEKVVREQPGSNLAKKALEIILRAAEELAKLPDPEALKEAVKAAEKVVREQPGSELAKKALEIIE |
| | RAAEELKKSPDPEAQKEAKKAEQKVREERPGGWLEHHHHHH (SEQ ID NO: 550) |
| DHR | MTTEDERRELEKVARKAIEAAREGNTDEVREQLQRALEIARESGTTEAVKLALEVVARVAIEAARRGNTDA |
| 54 | VREALEVALEIARESGTTEAVKLALEVVARVAIEAARRGNTDAVREALEVALEIARESGTEEAVRLALEVV |
| | KRVSDEAKKQGNEDAVKEAEEVRKKIEEESGGWLEHHHHHH (SEQ ID NO: 551) |
| DHR | MSSVAEEIEKRAKKISKELKKEGKNPEWIEELQRAADKLVEVARRATSSDALEIAKRAVKIAEELAKQGSN |
| 55 | PKWIAELLKAAAKLVEVAARATSSDALEIAKRAVKIAEELAKQGSNPKWIAELLKAAAKLVEVAARATSPK |
| | ALKQAKEAVKEAEELAKKGRNPKEIAEELKKRAKEVEKLARSTGWLEHHHHHH (SEQ ID NO: 552) |
| DHR | MSSVAEEIEKRCKKISKELKKEGKNPEWIEELQRACDKLVEVARRATSSDALEIAKRCVKIAEELAKQGSN |
| 56 | PKWIAELLKACAKLVEVAARATSSDALEIAKRCVKIAEELAKQGSNPKWIAELLKACAKLVEVAARATSPK |
| | ALKOAKECVKEAEELAKKGRNPKEIAEELKKCAKEVEKLARSTGWLEHHHHHH (SEQ ID NO: 553) |
| DHR | MSTEELKKVLERVRELSERAKESTDPEEALKIAKEVIELALKAVKEDPSTDALRAVLEAVRLASEVAKRVT |
| 57 | DPDKALKIAKLVIELALEAVKEDPSTDALRAVLEAVRLASEVAKRVTDPDKALKIAKLVIELALEAVKEDP |
| | SEEAKRAVEEAKRLAEEVSKRVTDPELSEKIRQLVKELEEEAQKEDPGWLEHHHHHH (SEQ ID NO: |
| | 554) |
| DHR | MSTEELKKVLERVRELCERAKESTDPEEALKIAKEVIELALKAVKEDPSTDALRAVLEAVRCACEVAKRVT |
| 58 | DPDKALKIAKLVIELALEAVKEDPSTDALRAVLEAVRCACEVAKRVTDPDKALKIAKLVIELALEAVKEDP |
| | SEEAKRAVEEAKRCAEEVSKRVTDPELSEKIRQLVKELEEEAQKEDPGWLEHHHHHH (SEQ ID NO: |
| | 555) |
| DHR | MKTEVEKKAKEVIKEAKELAKELDSEEAKKVVERIKEAAEAAKRAAEQGKTEVAKLALKVLEEAIELAKEN |
| 59 | RSEEALKVVLEIARAALAAAQAAEEGKTEVAKLALKVLEEAIELAKENRSEEALKVVLEIARAALAAAQAA |
| | EEGKSDEARDALRRLEEAIEEAKENRSKESLEKVREEAKEAEQQAEDAREGGWLEHHHHHH (SEQ ID |
| | NO: 556) |
| DHR | MTDIKKKAEEIIKEAKKOGSEDAIRLAQEAKKQGTDILVRAAEIVVRAQEQGSEDAIRLAKEASREGTDIL |
| 60 | VRAAEIVVRAQEQGSEDAIRLAKEASREGTPTLVKAAEKVVRAQQKGSQDTIEKAKEESREGGWLEHHHHH |
| | H (SEQ ID NO: 557) |
| DHR | MTDIKKKAEEIIKEAKKQGSEDAIRLAQECKKQGTDICVRAAEIVVRAQEQGSEDAIRLAKECSREGTDIC |
| 61 | VRAAEIVVRAQEQGSEDAIRLAKECSREGTPTCVKAAEKVVRAQQKGSQDTIEKAKEESREGGWLEHHHHH |
| | H (SEQ ID NO: 558) |
| DHR | MDNDEKRKRAEKALQRAQEAEKKGDVEEAVRAAQEAVRAAKESGDNDVLRKVAEQALRIAKEAEKQGNVEV |
| 62 | AVKAARVAVEAAKQAGDNDVLRKVAEQALRIAKEAEKQGNVEVAVKAARVAVEAAKQAGDQDVLRKVSEQA |
| | ERISKEAKKQGNSEVSEEARKVADEAKKQTGGWLEHHHHHH (SEQ ID NO: 559) |
| DHR | MDPDEDRERLKEELKKIREALREAKEKPDPEEIKRALREVLEAIRRILKLAERAGDPDLAREALKEINKVI |
| 63 | REALEIAKRVPDPEVIKEALRVVLEAIRAILKLAEQAGDPDLAREALKEINKVIREALEIAKRVPDPEVIK |
| | EALRVVLEAIRAILKLAEQAGDPDLAREALEEIDKVIDEAQEISERVPDEEVQREAQEVIKEADRARKKLS |
| | EQSGGWLEHHHHHH (SEQ ID NO: 560) |
| DHR | MDPEDELKRVEKLVKEAEELLRQAKEKGSEEDLEKALRTAEEAAREAKKVLEQAEKEGDPEVALRAVELVV |
| 64 | RVAELLLRIAKESGSEEALERALRVAEEAARLAKRVLELAEKQGDPEVALRAVELVVRVAELLLRIAKESG |
| | SEEALERALRVAEEAARLAKRVLELAEKQGDPEVARRAVELVKRVAELLERIARESGSEEAKERAERVREE |
| | ARELQERVKELREREGGWLEHHHHHH (SEQ ID NO: 561) |
| DHR | MDPEDELKRVEKLVKEAEELLROCKEKGSEECLEKALRTAEEAAREAKKVLEQAEKEGDPEVALRAVELVV |
| 65 | RVAELLLRICKESGSEECLERALRVAEEAARLAKRVLELAEKQGDPEVALRAVELVVRVAELLLRICKESG |
| | SEECLERALRVAEEAARLAKRVLELAEKQGDPEVARRAVELVKRVAELLERICRESGSEECKERAERVREE |
| | ARELQERVKELREREGGWLEHHHHHH (SEQ ID NO: 562) |
| DHR | MTSDDDKVREAEERVREAIERIQRALKKRDTPDARKALEAAKKLLKVVEKAKKRGTSDAIKVAEAAARVAE |
| 66 | AIARILEALNERDTPDARKALRAAIKLAEVVYKAAESGTSDAIKVAEAAARVAEAIARILEALNERDTPDA |
| | RKALRAAIKLAEVVYKAAESGTTEALKVAEKAARVAEKIARILEKLNERDTPEARKKLRQAIKEAEKVYKE |
| | SEQGGWLEHHHHHH (SEQ ID NO: 563) |
| DHR | MTSEIDKLIKKLRQTAKEVKREAEERKRRSTDPTVREVIERLAQLALDVAEEAARLIKKATTSEVAKLVWK |
| 67 | LARTAIEVIREAIERAERSTDPEVIRVILELARLAAEVAKEAARLIVKATTSEVAKLVWKLARTAIEVIRE |
| | AIERAERSTDPEVIRVILELARLAAEVAKEAARLIVKATTEEVAKKVWKEAYRAIEEIRKAIEKAERSTDP |
| | NEIKKILEEARKKAEEAIERAKEIVKSTGWLEHHHHHH (SEQ ID NO: 564) |
| DHR | MTPRERLEEAKERVEEIRELIDKARKLQEQGNKEEAEKVLREAREQIREVTRELEEIAKNSDTPELALRAA |
| 68 | ELLVRLIKLLIEIAKLLQEQGNKEEAEKVLREATELIKRVTELLEKIAKNSDTPELALRAAELLVRLIKLL |
| | IEIAKLLQEQGNKEEAEKVLREATELIKRVTELLEKIAKNSDTPELAKRAAELLKRLIELLKEIAKLLEEE |
| | GNEDEAEKVKEEAKELEERVRELEERIRKNSDGWLEHHHHHH (SEQ ID NO: 565) |
| DHR | MNPQEDLERAEKVVRSVEEVLQRAKEAQREGDKEKVERLIKEAENQIRKARELLERVVRONPDDPEVLLRV |
| 69 | AELIVRLVEVVLELAKLAEKNGDKEQVERLIQTAEELIREARELLERVSREIPDNPEVLLRVAELIVRLVE |
| | VVLELAKLAEKNGDKEQVERLIQTAEELIREARELLERVSREIPDNPESLKRVAELIKRLVKVVDELSKLA |
| | ERNGDRDQVERLROLAEELRREAEELEERVRRERPDGWLEHHHHHH (SEQ ID NO: 566) |
| DHR | MSTEEKIEEARQSIKEAERSLREGNPEKAREDVRRALELVRELEKLARKTGSTEVLIEAARLAIEVARVAL |
| 70 | KVGSPETAREAVRTALELVQELERQARKTGSTEVLIEAARLAIEVARVALKVGSPETAREAVRTALELVQE |
| | LERQARKTGSDEVLKRAAELAKEVARVAKEVGSPETARQARETAERLREELRRNREKKGGWLEHHHHHH |
| | (SEQ ID NO: 567) |
| DHR | MDPEEILERAKESLERAREASERGDEEEFRKAAEKALELAKRLVEQAKKEGDPELVLEAAKVALRVAELAA |
| 71 | KNGDKEVFKKAAESALEVAKRLVEVASKEGDPELVLEAAKVALRVAELAAKNGDKEVFKKAAESALEVAKR |
| | LVEVASKEGDPELVEEAAKVAEEVRKLAKKOGDEEVYEKARETAREVKEELKRVREEKGGWLEHHHHHH |
| | (SEQ ID NO: 568) |
| DHR | MDSTKEKARQLAEEAKETAEKVGDPELIKLAEQASQEGDSEKAKAILLAAEAARVAKEVGDPELIKLALEA |
| 72 | ARRGDSEKAKAILLAAEAARVAKEVGDPELIKLALEAARRGDSEKARAILEAAERAREAKERGDPEQIKKA |
| | RELAKRGGWLEHHHHHH (SEQ ID NO: 569) |
| DHR | MDAEEEAKEAIKRAQEAIELARKGNPEEARKVAEEARERAERVREEAEKRGDAEVLALVAIALALVAIALA |
| 73 | EVGNPEEAREVAERAKEIAERVRELAEKRGDAEVLALVAIALALVAIALAEVGNPEEAREVAERAKEIAER |
| | VRELAEKRGDARVLKLVAKALELVAEALKKVGNPEEAREVEERAREIKERVRRLLEEKGGWLEHHHHHH |
| | (SEQ ID NO: 570) |
| DHR | MDSEADRIIKKLQKEIKEVEQEARDSNDDEERELLKRLAEALKRAAEAVKRAQESGDSEAIRIIKKLVKEI |
| 74 | TEVVREARKSTDKEEIELLIRLAEALARAAEAVADAAKSGDSEAIRIIKKLVKEITEVVREARKSTDKEEI |
| | ELLIRLAEALARAAEAVADAAKSGDQEAIKRIKKLVKKIIEVVRKARKSTNKKEIEKLIRKAEKLARKAEQ |
| | IAEDAKRGGWLEHHHHHH (SEQ ID NO: 571) |
| DHR | MDSEKEKATELAERAQDVASRVEEEARREGSRELIEIARELRERAEEASQEGDSEKAKAILLAAKAVLVAV |
| 75 | EVYERAKROGSDELREIARELAKEALRAAQEGDSEKAKAILLAAKAVLVAVEVYERAKROGSDELREIARE |
| | LAKEALRAAQEGDSEKARAILEAAREVLRAVEQYERAKRRGDDDERERAREEAREALERAREGGWLEHHHH |
| | HH (SEQ ID NO: 572) |
| DHR | MNPELEEWIRRAKEVAKEVEKVAQRAEEEGNPDLRDSAKELRRAVEEAIEEAKKQGNPELVEWVARAAKVA |
| 76 | AEVIKVAIQAEKEGNRDLFRAALELVRAVIEAIEEAVKQGNPELVEWVARAAKVAAEVIKVAIQAEKEGNR |
| | DLFRAALELVRAVIEAIEEAVKOGNPELVERVARLAKKAAELIKRAIRAEKEGNRDERREALERVREVIER |
| | IEELVRQGGWLEHHHHHH (SEQ ID NO: 573) |
| DHR | MNSDEEEAREWAERAEEAAKEALEQAKREGDEDARRVAEELEKQAEEARRKKDSEEAEAVYWAARAVLAAL |
| 77 | EALEQAKREGDEDARRVAEELLRQAEEAARKKNSEEAEAVYWAARAVLAALEALEQAKREGDEDARRVAEE |
| | LLRQAEEAARKKNPEEARAVYEAARDVLEALQRLEEAKRRGDEEERREAEERLRQAEERARKKGWLEHHHH |
| | HH (SEQ ID NO: 574) |
| DHR | MNSDEEEAREWAERAEEAAKEALEQAKREGDEDARRCAEELEKQAEEARRKKDSEEAEAVYWAARAVLAAL |
| 78 | EALEQAKREGDEDARRCAEELLRQACEAARKKNSEEAEAVYWAARAVLAALEALEQAKREGDEDARRCAEE |
| | LLRQACEAARKKNPEEARAVYEAARDVLEALQRLEEAKRRGDEEERREAEERLRQACERARKKGWLEHHHH |
| | HH (SEQ ID NO: 575) |
| DHR | MSSDEEEARELIERAKEAAERAQEAAERTGDPRVRELARELKRLAQEAAEEVKRDPSSSDVNEALKLIVEA |
| 79 | IEAAVRALEAAERTGDPEVRELARELVRLAVEAAEEVQRNPSSSDVNEALKLIVEAIEAAVRALEAAERTG |
| | DPEVRELARELVRLAVEAAEEVQRNPSSEEVNEALKKIVKAIQEAVESLREAEESGDPEKREKARERVREA |
| | VERAEEVQRDPSGWLEHHHHHH (SEQ ID NO: 576) |
| DHR | MNSEELERESEEAERRLQEARKRSEEARERGDLKELAEALIEEARAVQELARVASERGNSEEAERASEKAQ |
| 80 | RVLEEARKVSEEAREQGDDEVLALALIAIALAVLALAEVASSRGNSEEAERASEKAQRVLEEARKVSEEAR |
| | EQGDDEVLALALIAIALAVLALAEVASSRGNKEEAERAYEDARRVEEEARKVKESAEEQGDSEVKRLAEEA |
| | EQLAREARRHVQETRGGWLEHHHHHH (SEQ ID NO: 577) |
| DHR | MNSEELERESEEAERRLQEARKRSEEARERGDLKELAEALIEEARAVQELARVACERGNSEEAERASEKAQ |
| 81 | RVLEEARKVSEEAREQGDDEVLALALIAIALAVLALAEVACCRGNSEEAERASEKAQRVLEEARKVSEEAR |
| | EQGDDEVLALALIAIALAVLALAEVACCRGNKEEAERAYEDARRVEEEARKVKESAEEQGDSEVKRLAEEA |
| | EQLAREARRHVQECRGGWLEHHHHHH (SEQ ID NO: 578) |
| DHR | MNDEEVQEAVERAEELREEAEELIKKARKTGDPELLRKALEALEEAVRAVEEAIKRNPDNDEAVETAVRLA |
| 82 | RELKKVAEELQERAKKTGDPELLKLALRALEVAVRAVELAIKSNPDNDEAVETAVRLARELKKVAEELQER |
| | AKKTGDPELLKLALRALEVAVRAVELAIKSNPDNEEAVETAKRLAEELRKVAELLEERAKETGDPELQELA |
| | KRAKEVADRARELAKKSNPNGWLEHHHHHH (SEQ ID NO: 579) |
| DHR | MNDEEVQEACERAEELREEAEELIKKARKTGDPELLRKALEALEEAVRAVEEAIKRNPDNDECVETACRLA |
| 83 | RELKKVAEELQERAKKTGDPELLKLALRALEVAVRAVELAIKSNPDNDECVETACRLARELKKVAEELQER |
| | AKKTGDPELLKLALRALEVAVRAVELAIKSNPDNEECVETAKRLAEELRKVAELLEERAKETGDPELQELA |
| | KRAKEVADRARELAKKSNPNGWLEHHHHHH (SEQ ID NO: 580) |

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The above definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of skill in the art, such as the Oxford Dictionary of Biochemistry and Molecular Biology (Ed. Anthony Smith, Oxford University Press, Oxford, 2004).

As used herein and unless otherwise indicated, the terms “a” and “an” are taken to mean “one”, “at least one” or “one or more”. Unless otherwise required by context, singular terms used herein shall include pluralities and plural terms shall include the singular. Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

The above description provides specific details for a thorough understanding of, and enabling description for, embodiments of the disclosure. However, one skilled in the art will understand that the disclosure may be practiced without these details. In other instances, well-known structures and functions have not been shown or described in detail to avoid unnecessarily obscuring the description of the embodiments of the disclosure. The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### REFERENCES

1. Kajava, A. V. Tandem repeats in proteins: From sequence to structure. J. Struct. Biol. 179, 279-288 (2012).
2. Marcotte, E. M., Pellegrini, M., Yeates, T. O. & Eisenberg, D. A census of protein repeats1. J. Mol. Biol. 293, 151-160 (1999).
3. Binz, H. K. et al. High-affinity binders selected from designed ankyrin repeat protein libraries. Nat. Biotechnol. 22, 575-582 (2004).
4. Varadamsetty, G., Tremmel, D., Hansen, S., Parmeggiani, F. & Plückthun, A. Designed Armadillo Repeat Proteins: Library Generation, Characterization and Selection of Peptide Binders with High Specificity. J. Mol. Biol. 424, 68-87 (2012).
5. Cortajarena, A. L., Liu, T. Y., Hochstrasser, M. & Regan, L. Designed Proteins To Modulate Cellular Networks. ACS Chem. Biol. 5, 545-552 (2010).
6. Kobe, B. & Kajava, A. V. When protein folding is simplified to protein coiling: the continuum of solenoid protein structures. Trends Biochem. Sci. 25, 509-515 (2000).
7. Wetzel, S. K., Settanni, G., Kenig, M., Binz, H. K. & Plückthun, A. Folding and Unfolding Mechanism of Highly Stable Full-Consensus Ankyrin Repeat Proteins. J. Mol. Biol. 376, 241-257 (2008).
8. Cortajarena, A. L. & Regan, L. calorimetric study of a series of designed repeat proteins: Modular structure and modular folding. Protein Sci. 20, 336-340 (2011).
9. Binz, H. K., Stumpp, M. T., Forrer, P., Amstutz, P. & Plückthun, A. Designing Repeat Proteins: Well-expressed, Soluble and Stable Proteins from Combinatorial Libraries of Consensus Ankyrin Repeat Proteins. J. Mol. Biol. 332, 489-503 (2003).
10. Mosavi, L. K., Minor, D. L. & Peng, Z. Consensus-derived structural determinants of the ankyrin repeat motif. Proc. Natl. Acad. Sci. 99, 16029-16034 (2002).
11. Main, E. R. G., Xiong, Y., Cocco, M. J., D'Andrea, L. & Regan, L. Design of Stable α-Helical Arrays from an Idealized TPR Motif. Structure 11, 497-508 (2003).
12. Urvoas, A. et al. Design, Production and Molecular Structure of a New Family of Artificial Alpha-helicoidal Repeat Proteins (αRep) Based on Thermostable HEAT-like Repeats. J. Mol. Biol. 404, 307-327 (2010).
13. Lee, S.-C. et al. Design of a binding scaffold based on variable lymphocyte receptors of jawless vertebrates by module engineering. Proc. Natl. Acad. Sci. 109, 3299-3304 (2012).
14. Parmeggiani, F. et al. Designed Armadillo Repeat Proteins as General Peptide-Binding Scaffolds: Consensus Design and Computational Optimization of the Hydrophobic Core. J. Mol. Biol. 376, 1282-1304 (2008).
15. Yadid, I. & Tawfik, D. S. Reconstruction of Functional β-Propeller Lectins via Homo-oligomeric Assembly of Shorter Fragments. J. Mol. Biol. 365, 10-17 (2007).
16. Coquille, S. et al. An artificial PPR scaffold for programmable RNA recognition. Nat. Commun. 5, (2014).
17. Rämisch, S., Weininger, U., Martinsson, J., Akke, M. & André, I. Computational design of a leucine-rich repeat protein with a predefined geometry. Proc. Natl. Acad. Sci. 111, 17875-17880 (2014).
18. Lee, J. & Blaber, M. Experimental support for the evolution of symmetric protein architecture from a simple peptide motif. Proc. Natl. Acad. Sci. 108, 126-130 (2011).
19. Voet, A. R. D. et al. Computational design of a self-assembling symmetrical β-propeller protein. Proc. Natl. Acad. Sci. 111, 15102-15107 (2014).
20. Parmeggiani, F. et al. A General Computational Approach for Repeat Protein Design. J. Mol. Biol. 427, 563-575 (2015).
21. Tripp, K. W. & Barrick, D. Enhancing the Stability and Folding Rate of a Repeat Protein through the Addition of Consensus Repeats. J. Mol. Biol. 365, 1187-1200 (2007).
22. Park, K. et al. Control of repeat-protein curvature by computational protein design. Nat. Struct. Mol. Biol. 22, 167-174 (2015).
23. Huang, P.-S. et al. RosettaRemodel: A Generalized Framework for Flexible Backbone Protein Design. PLoS ONE 6, e24109 (2011).
24. Leaver-Fay, A. et al. ROSETTA3: an object-oriented software suite for the simulation and design of macromolecules. Methods Enzymol. 487, 545-574 (2011).
25. Huang, P.-S. et al. High thermodynamic stability of parametrically designed helical bundles. Science 346, 481-485 (2014).
26. Bradley, P., Misura, K. M. S. & Baker, D. Toward High-Resolution de Novo Structure Prediction for Small Proteins. Science 309, 1868-1871 (2005).
27. Rambo, R. P. & Tainer, J. A. Super-Resolution in Solution X-Ray Scattering and Its Applications to Structural Systems Biology. Annu. Rev. Biophys. 42, 415-441 (2013).
28. Hura, G. L. et al. Robust, high-throughput solution structural analyses by small angle X-ray scattering (SAXS). Nat. Methods 6, 606-612 (2009).
29. Hura, G. L. et al. Comprehensive macromolecular conformations mapped by quantitative SAXS analyses. Nat. Methods 10, 453-454 (2013).
30. Altschul, S. F. et al. Gapped BLAST and PSI-BLAST: a new generation of protein database search programs. Nucleic Acids Res. 25, 3389-3402 (1997).
31. Camacho, C. et al. BLAST+: architecture and applications. BMC Bioinformatics 10, 421 (2009).
32. Remmert, M., Biegert, A., Hauser, A. & Söding, J. HHblits: lightning-fast iterative protein sequence searching by HMM-HMM alignment. Nat. Methods 9, 173-175 (2012).
33. Punta, M. et al. The Pfam protein families database. Nucleic Acids Res. 40, D290-D301 (2012).
34. Waterhouse, A. M., Procter, J. B., Martin, D. M. A., Clamp, M. & Barton, G. J. Jalview Version 2—a multiple sequence alignment editor and analysis workbench. Bioinformatics 25, 1189-1191 (2009).
35. Zhang, Y. & Skolnick, J. TM-align: a protein structure alignment algorithm based on the TM-score. Nucleic Acids Res. 33, 2302-2309 (2005).
36. Di Domenico, T. et al. RepeatsDB: a database of tandem repeat protein structures. Nucleic Acids Res. 42, D352-D357 (2014).
37. Kabsch, W. XDS. Acta Crystallogr. Sect. D 66, 125-132 (2010).
38. Adams, P. D. et al. PHENIX: building new software for automated crystallographic structure determination. Acta Crystallogr. Sect. D 58, 1948-1954 (2002).
39. Emsley, P. & Cowtan, K. Coot: model-building tools for molecular graphics. Acta Crystallogr. D Biol. Crystallogr. 60, 2126-2132 (2004).
40. Chen, V. B. et al. MolProbity: all-atom structure validation for macromolecular crystallography. Acta Crystallogr. Sect. D 66, 12-21 (2010).
41. Classen, S. et al. Implementation and performance of SIBYLS: a dual endstation small-angle X-ray scattering and macromolecular crystallography beamline at the Advanced Light Source. J. Appl. Crystallogr. 46, 1-13 (2013).
42. Classen, S. et al. Software for the high-throughput collection of SAXS data using an enhanced Blu-Ice/DCS control system. J. Synchrotron Radiat. 17, 774-781 (2010).
43. Schneidman-Duhovny, D., Hammel, M., Tainer, J. A. & Sali, A. Accurate SAXS Profile Computation and its Assessment by Contrast Variation Experiments. Biophys. 1 105, 962-974 (2013).
44. Schneidman-Duhovny, D., Hammel, M. & Sali, A. FoXS: a web server for rapid computation and fitting of SAXS profiles. Nucleic Acids Res. 38, W540-W544 (2010).
45. Svergun, D., Barberato, C. & Koch, M. H. J. CRYSOL—a Program to Evaluate X-ray Solution Scattering of Biological Macromolecules from Atomic Coordinates. J. Appl. Crystallogr. 28, 768-773 (1995).
46. Petoukhov, M. V. et al. New developments in the ATSAS program package for small-angle scattering data analysis. J. Appl. Crystallogr. 45, 342-350 (2012).

We claim:

1. A polypeptide comprising or consisting of the amino acid sequence selected from the group consisting of:
(a) SEQ ID NO:1-[SEQ ID NO:2]_{(0 or 2-19)}-SEQ ID NO:3;
(b) SEQ ID NO:7-[SEQ ID NO:8]_{(0 or 2-19)}-SEQ ID NO:9;
(c) SEQ ID NO:13-[SEQ ID NO:14]_{(0 or 2-19)}-SEQ ID NO:15;
(d) SEQ ID NO:19-[SEQ ID NO:20]_{(0 or 2-19)}-SEQ ID NO:21;
(e) SEQ ID NO:25-[SEQ ID NO:26]_{(0 or 2-19)}-SEQ ID NO:27;
(f) SEQ ID NO:31-[SEQ ID NO:32]_{(0 or 2-19)}-SEQ ID NO:33;
(g) SEQ ID NO:37-[SEQ ID NO:38]_{(0 or 2-19)}-SEQ ID NO:39;
(h) SEQ ID NO:43-[SEQ ID NO:44]_{(0 or 2-19)}-SEQ ID NO:45;
(i) SEQ ID NO:49-[SEQ ID NO:50]_{(0 or 2-19)}-SEQ ID NO:51;
(j) SEQ ID NO:55-[SEQ ID NO:56]_{(0 or 2-19)}-SEQ ID NO:57;
(k) SEQ ID NO:61-[SEQ ID NO:62]_{(0 or 2-19)}-SEQ ID NO:63;
(l) SEQ ID NO:67-[SEQ ID NO:68]_{(0 or 2-19)}-SEQ ID NO:69;
(m) SEQ ID NO:73-[SEQ ID NO:74]_{(0 or 2-19)}-SEQ ID NO:75;
(n) SEQ ID NO:79-[SEQ ID NO:80]_{(0 or 2-19)}-SEQ ID NO:81;
(o) SEQ ID NO:85-[SEQ ID NO:86]_{(0 or 2-19)}-SEQ ID NO:87;
(p) SEQ ID NO:91-[SEQ ID NO:92]_{(0 or 2-19)}-SEQ ID NO:93;
(q) SEQ ID NO:97-[SEQ ID NO:98]_{(0 or 2-19)}-SEQ ID NO:99;
(r) SEQ ID NO:103-[SEQ ID NO:104]_{(0 or 2-19)}-SEQ ID NO:105;
(s) SEQ ID NO:109-[SEQ ID NO:110]_{(0 or 2-19)}-SEQ ID NO:111;
(t) SEQ ID NO:115-[SEQ ID NO:116]_{(0 or 2-19)}-SEQ ID NO:117;
(u) SEQ ID NO:121-[SEQ ID NO:122]_{(0 or 2-19)}-SEQ ID NO:123;
(v) SEQ ID NO:127-[SEQ ID NO:128]_{(0 or 2-19)}-SEQ ID NO:129;
(w) SEQ ID NO:133-[SEQ ID NO:134]_{(0 or 2-19)}-SEQ ID NO:135;
(x) SEQ ID NO:139-[SEQ ID NO:140]_{(0 or 2-19)}-SEQ ID NO:141;
(y) SEQ ID NO:145-[SEQ ID NO:146]_{(0 or 2-19)}-SEQ ID NO:147;
(z) SEQ ID NO:151-[SEQ ID NO:152]_{(0 or 2-19)}-SEQ ID NO:153;
(aa) SEQ ID NO:157-[SEQ ID NO:158]_{(0 or 2-19)}-SEQ ID NO:159;
(bb) SEQ ID NO:163-[SEQ ID NO:164]_{(0 or 2-19)}-SEQ ID NO:165;
(cc) SEQ ID NO:169-[SEQ ID NO:170]_{(0 or 2-19)}-SEQ ID NO:171;
(dd) SEQ ID NO:175-[SEQ ID NO:176]_{(0 or 2-19)}-SEQ ID NO:177;
(ee) SEQ ID NO:181-[SEQ ID NO:182]_{(0 or 2-19)}-SEQ ID NO:183;
(ff) SEQ ID NO:187-[SEQ ID NO:188]_{(0 or 2-19)}-SEQ ID NO:189;
(gg) SEQ ID NO:193-[SEQ ID NO:194]_{(0 or 2-19)}-SEQ ID NO:195;
(hh) SEQ ID NO:199-[SEQ ID NO:200]_{(0 or 2-19)}-SEQ ID NO:201;
(ii) SEQ ID NO:205-[SEQ ID NO:206]_{(0 or 2-19)}-SEQ ID NO:207;
(jj) SEQ ID NO:211-[SEQ ID NO:212]_{(0 or 2-19)}-SEQ ID NO:213;
(kk) SEQ ID NO:217-[SEQ ID NO:218]_{(0 or 2-19)}-SEQ ID NO:219;
(ll) SEQ ID NO:223-[SEQ ID NO:224]_{(0 or 2-19)}-SEQ ID NO:225;
(mm) SEQ ID NO:229-[SEQ ID NO:230]_{(0 or 2-19)}-SEQ ID NO:231;
(nn) SEQ ID NO:235-[SEQ ID NO:236]_{(0 or 2-19)}-SEQ ID NO:237;
(oo) SEQ ID NO:241-[SEQ ID NO:242]_{(0 or 2-19)}-SEQ ID NO:243;
(pp) SEQ ID NO:247-[SEQ ID NO:248]_{(0 or 2-19)}-SEQ ID NO:249;
(qq) SEQ ID NO:253-[SEQ ID NO:254]_{(0 or 2-19)}-SEQ ID NO:255;
(rr) SEQ ID NO:259-[SEQ ID NO:260]_{(0 or 2-19)}-SEQ ID NO:261;
(ss) SEQ ID NO:265-[SEQ ID NO:266]_{(0 or 2-19)}-SEQ ID NO:267;
(tt) SEQ ID NO:271-[SEQ ID NO:272]_{(0 or 2-19)}-SEQ ID NO:273;
(uu) SEQ ID NO:277-[SEQ ID NO:278]_{(0 or 2-19)}-SEQ ID NO:278;
(vv) SEQ ID NO:283-[SEQ ID NO:284]_{(0 or 2-19)}-SEQ ID NO:285;
(ww) SEQ ID NO:289-[SEQ ID NO:290]_{(0 or 2-19)}-SEQ ID NO:291;
(xx) SEQ ID NO:295-[SEQ ID NO:296]_{(0 or 2-19)}-SEQ ID NO:297;
(yy) SEQ ID NO:301-[SEQ ID NO:302]_{(0 or 2-19)}-SEQ ID NO:303;
(zz) SEQ ID NO:307-[SEQ ID NO:308]_{(0 or 2-19)}-SEQ ID NO:309;
(aaa) SEQ ID NO:313-[SEQ ID NO:314]_{(0 or 2-19)}-SEQ ID NO:315;
(bbb) SEQ ID NO:319-[SEQ ID NO:320]_{(0 or 2-19)}-SEQ ID NO:321;
(ccc) SEQ ID NO:325-[SEQ ID NO:326]_{(0 or 2-19)}-SEQ ID NO:327;
(ddd) SEQ ID NO:331-[SEQ ID NO:332]_{(0 or 2-19)}-SEQ ID NO:333;
(eee) SEQ ID NO:337-[SEQ ID NO:338]_{(0 or 2-19)}-SEQ ID NO:339;
(fff) SEQ ID NO:343-[SEQ ID NO:344]_{(0 or 2-19)}-SEQ ID NO:345;
(ggg) SEQ ID NO:349-[SEQ ID NO:350]_{(0 or 2-19)}-SEQ ID NO:351;
(hhh) SEQ ID NO:355-[SEQ ID NO:356]_{(0 or 2-19)}-SEQ ID NO:357;
(iii) SEQ ID NO:361-[SEQ ID NO:362]_{(0 or 2-19)}-SEQ ID NO:363;
(jjj) SEQ ID NO:367-[SEQ ID NO:368]_{(0 or 2-19)}-SEQ ID NO:369;
(kkk) SEQ ID NO:373-[SEQ ID NO:374]_{(0 or 2-19)}-SEQ ID NO:375;
(lll) SEQ ID NO:379-[SEQ ID NO:380]_{(0 or 2-19)}-SEQ ID NO:381;
(mmm) SEQ ID NO:385-[SEQ ID NO:386]_{(0 or 2-19)}-SEQ ID NO:387;
(nnn) SEQ ID NO:391-[SEQ ID NO:392]_{(0 or 2-19)}-SEQ ID NO:393;
(ooo) SEQ ID NO:397-[SEQ ID NO:398]_{(0 or 2-19)}-SEQ ID NO:399;
(ppp) SEQ ID NO:403-[SEQ ID NO:404]_{(0 or 2-19)}-SEQ ID NO:405; and
(qqq) SEQ ID NO:409-[SEQ ID NO:410]_{(0 or 2-19)}-SEQ ID NO:411;
wherein the domain in brackets is an optional internal domain.

2. The polypeptide of claim 1, wherein the polypeptide comprises or consists of the amino acid sequence selected from the group consisting of:
(A) SEQ ID NO:4-[SEQ ID NO:5]_{(0 or 2-19)}-SEQ ID NO:6;
(B) SEQ ID NO:10-[SEQ ID NO:11]_{(0 or 2-19)}-SEQ ID NO:12;
(C) SEQ ID NO:16-[SEQ ID NO:17]_{(0 or 2-19)}-SEQ ID NO:18;
(D) SEQ ID NO:22-[SEQ ID NO:23]_{(0 or 2-19)}-SEQ ID NO:24;
(E) SEQ ID NO:28-[SEQ ID NO:29]_{(0 or 2-19)}-SEQ ID NO:30;
(F) SEQ ID NO:34-[SEQ ID NO:35]_{(0 or 2-19)}-SEQ ID NO:36;
(G) SEQ ID NO:40-[SEQ ID NO:41]_{(0 or 2-19)}-SEQ ID NO:42;
(H) SEQ ID NO:46-[SEQ ID NO:47]_{(0 or 2-19)}-SEQ ID NO:48;
(I) SEQ ID NO:52-[SEQ ID NO:53]_{(0 or 2-19)}-SEQ ID NO:54;
(J) SEQ ID NO:58-[SEQ ID NO:59]_{(0 or 2-19)}-SEQ ID NO:60;
(K) SEQ ID NO:64-[SEQ ID NO:65]_{(0 or 2-19)}-SEQ ID NO:66;
(L) SEQ ID NO:70-[SEQ ID NO:71]_{(0 or 2-19)}-SEQ ID NO:72;
(M) SEQ ID NO:76-[SEQ ID NO:77]_{(0 or 2-19)}-SEQ ID NO:78;
(N) SEQ ID NO:82-[SEQ ID NO:83]_{(0 or 2-19)}-SEQ ID NO:84;
(O) SEQ ID NO:88-[SEQ ID NO:89]_{(0 or 2-19)}-SEQ ID NO:90;
(P) SEQ ID NO:94-[SEQ ID NO:95]_{(0 or 2-19)}-SEQ ID NO:96;
(Q) SEQ ID NO:100-[SEQ ID NO:101]_{(0 or 2-19)}-SEQ ID NO:102;
(R) SEQ ID NO:106-[SEQ ID NO:107]_{(0 or 2-19)}-SEQ ID NO:108;
(S) SEQ ID NO:112-[SEQ ID NO:113]_{(0 or 2-19)}-SEQ ID NO:114;
(T) SEQ ID NO:118-[SEQ ID NO:119]_{(0 or 2-19)}-SEQ ID NO:120;
(U) SEQ ID NO:124-[SEQ ID NO:125]_{(0 or 2-19)}-SEQ ID NO:126;
(V) SEQ ID NO:130-[SEQ ID NO:131]_{(0 or 2-19)}-SEQ ID NO:132;
(W) SEQ ID NO:136-[SEQ ID NO:137]_{(0 or 2-19)}-SEQ ID NO:138;
(X) SEQ ID NO:142-[SEQ ID NO:143]_{(0 or 2-19)}-SEQ ID NO:144;
(Y) SEQ ID NO:148-[SEQ ID NO:149]_{(0 or 2-19)}-SEQ ID NO:150;
(Z) SEQ ID NO:154-[SEQ ID NO:155]_{(0 or 2-19)}-SEQ ID NO:156;
(AA) SEQ ID NO: 160-[SEQ ID NO:161]_{(0 or 2-19)}-SEQ ID NO:162;
(BB) SEQ ID NO: 166-[SEQ ID NO:167]_{(0 or 2-19)}-SEQ ID NO:168;
(CC) SEQ ID NO: 172-[SEQ ID NO: 173]_{(0 or 2-19)}-SEQ ID NO:174;
(DD) SEQ ID NO: 178-[SEQ ID NO:179]_{(0 or 2-19)}-SEQ ID NO:180;
(EE) SEQ ID NO: 184-[SEQ ID NO:185]_{(0 or 2-19)}-SEQ ID NO:186;
(FF) SEQ ID NO: 190-[SEQ ID NO:191]_{(0 or 2-19)}-SEQ ID NO:192;
(GG) SEQ ID NO: 196-[SEQ ID NO:197]_{(0 or 2-19)}-SEQ ID NO:198;
(HH) SEQ ID NO:202-[SEQ ID NO:203]_{(0 or 2-19)}-SEQ ID NO:204;
(II) SEQ ID NO:208-[SEQ ID NO:209]_{(0 or 2-19)}-SEQ ID NO:210;
(JJ) SEQ ID NO:214-[SEQ ID NO:215]_{(0 or 2-19)}-SEQ ID NO:216;
(KK) SEQ ID NO:220-[SEQ ID NO:221]_{(0 or 2-19)}-SEQ ID NO:222;
(LL) SEQ ID NO:226-[SEQ ID NO:227]_{(0 or 2-19)}-SEQ ID NO:228;
(MM) SEQ ID NO:232-[SEQ ID NO:233]_{(0 or 2-19)}-SEQ ID NO:234;
(NN) SEQ ID NO:238-[SEQ ID NO:239]_{(0 or 2-19)}-SEQ ID NO:240;
(OO) SEQ ID NO:244-[SEQ ID NO:245]_{(0 or 2-19)}-SEQ ID NO:246;
(PP) SEQ ID NO:250-[SEQ ID NO:251]_{(0 or 2-19)}-SEQ ID NO:252;
(QQ) SEQ ID NO:256-[SEQ ID NO:257]_{(0 or 2-19)}-SEQ ID NO:258;
(RR) SEQ ID NO:262-[SEQ ID NO:263]_{(0 or 2-19)}-SEQ ID NO:264;
(SS) SEQ ID NO:268-[SEQ ID NO:269]_{(0 or 2-19)}-SEQ ID NO:270;
(TT) SEQ ID NO:274-[SEQ ID NO:275]_{(0 or 2-19)}-SEQ ID NO:276;
(UU) SEQ ID NO:280-[SEQ ID NO:281]_{(0 or 2-19)}-SEQ ID NO:282;
(VV) SEQ ID NO:286-[SEQ ID NO:287]_{(0 or 2-19)}-SEQ ID NO:288;
(WW) SEQ ID NO:292-[SEQ ID NO:293]_{(0 or 2-19)}-SEQ ID NO:294;
(XX) SEQ ID NO:298-[SEQ ID NO:299]_{(0 or 2-19)}-SEQ ID NO:300;
(YY) SEQ ID NO:304-[SEQ ID NO:305]_{(0 or 2-19)}-SEQ ID NO:306;
(ZZ) SEQ ID NO:310-[SEQ ID NO:311]_{(0 or 2-19)}-SEQ ID NO:312;
(AAA) SEQ ID NO:316-[SEQ ID NO:317]_{(0 or 2-19)}-SEQ ID NO:318;
(BBB) SEQ ID NO:322-[SEQ ID NO:323]_{(0 or 2-19)}-SEQ ID NO:324;
(CCC) SEQ ID NO:328-[SEQ ID NO:329]_{(0 or 2-19)}-SEQ ID NO:330;
(DDD) SEQ ID NO:334-[SEQ ID NO:335]_{(0 or 2-19)}-SEQ ID NO:336;
(EEE) SEQ ID NO:340-[SEQ ID NO:341]_{(0 or 2-19)}-SEQ ID NO:342;
(FFF) SEQ ID NO:346-[SEQ ID NO:347]_{(0 or 2-19)}-SEQ ID NO:348;
(GGG) SEQ ID NO:352-[SEQ ID NO:353]_{(0 or 2-19)}-SEQ ID NO:354;
(HHH) SEQ ID NO:358-[SEQ ID NO:359]_{(0 or 2-19)}-SEQ ID NO:360;
(III) SEQ ID NO:364-[SEQ ID NO:365]_{(0 or 2-19)}-SEQ ID NO:366;
(JJJ) SEQ ID NO:370-[SEQ ID NO:371]_{(0 or 2-19)}-SEQ ID NO:372;
(KKK) SEQ ID NO:376-[SEQ ID NO:377]_{(0 or 2-19)}-SEQ ID NO:378;
(LLL) SEQ ID NO:382-[SEQ ID NO:383]_{(0 or 2-19)}-SEQ ID NO:384;
(MMM) SEQ ID NO:388-[SEQ ID NO:389]_{(0 or 2-19)}-SEQ ID NO:390;
(NNN) SEQ ID NO:394-[SEQ ID NO:395]_{(0 or 2-19)}-SEQ ID NO:396;
(OOO) SEQ ID NO:400-[SEQ ID NO:401]_{(0 or 2-19)}-SEQ ID NO:402;
(PPP) SEQ ID NO:406-[SEQ ID NO:407]_{(0 or 2-19)}-SEQ ID NO:408; and
(QQQ) SEQ ID NO:412-[SEQ ID NO:413]_{(0 or 2-19)}-SEQ ID NO:414;
wherein the domain in brackets is an optional internal domain.

3. The polypeptide of claim 1, wherein the optional internal domain is absent.

4. The polypeptide of claim 1, wherein the optional internal domain is present in 2-19 copies.

5. The polypeptide of claim 1, wherein the optional internal domain is present in 2-3 copies.

6. A polypeptide comprising or consisting of a polypeptide having at least 50% identity over its length with the amino acid sequence selected from the group consisting of SEQ ID NO: 415-497.

7. The polypeptide of claim 6, comprising or consisting of a polypeptide having at least 75% identity over its length with the amino acid sequence selected from the group consisting of SEQ ID NO: 415-497.

8. The polypeptide of claim 6, comprising or consisting of a polypeptide having at least 90% identity over its length with the amino acid sequence selected from the group consisting of SEQ ID NO: 415-497.

9. The polypeptide of claim 6, comprising or consisting of the amino acid sequence selected from the group consisting of SEQ ID NO: 415-497.

10. A protein assembly comprising a plurality of polypeptides having the same amino acid sequence selected from the group listed in claim 1.

11. A recombinant nucleic acid encoding a polypeptide claim 1.

12. A recombinant expression vector comprising the nucleic acid of claim 11 operatively linked to a promoter.

13. A recombinant host cell comprising the recombinant expression vectors of claim 12.
